(19) United States
(12) Patent Application Publication
(10) Pub. No.: US 2026/0152550 A1
(43) Pub. Date: Jun. 4, 2026

(54) Treatment of diuretic resistant heart failure patients having at least one copy of the TMPRSS6 rs855791 allele

(71) Applicants: Yale University, New Haven, CT (US); Novo Nordisk A/S, Bagsvaerd (DK)

(72) Inventors: Jeffrey Testani, Guilford, CT (US); Veena Rao, Derby, CT (US); Rahul Kakkar, Weston, MA (US); Madhav N. Devalaraja, Acton, MA (US); Chih-Hung Lo, Rockville, MD (US)

(21) Appl. No.: 19/398,713

(22) Filed: Nov. 24, 2025

Related U.S. Application Data
(63) Continuation of application No. 17/519,082, filed on Nov. 4, 2021, now Pat. No. 12,600,773.
Application No. 17/519,082 is a continuation of application No. 16/482,038, now Pat. No. 11,203,636.
(60) Provisional application No. 62/453,257, filed on Feb. 1, 2017.

(51) Int. Cl. C07K 16/24

(52) U.S. Cl. CPC C07K 16/248; C07K 2317/76

(57) ABSTRACT

The present disclosure provides methods of treating diuretic resistance by administering an IL-6 antagonist to patients who require diuresis. In typical embodiments, the patient has heart failure. Optionally, the patient has elevated urine levels of IL-6, plasma levels of IL-6, or both urine and plasma levels of IL-6.

### CROSS-REFERENCE TO RELATED APPLICATIONS

This application is a continuation of U.S. application Ser. No. 17/519,082, filed Nov. 4, 2021, which is a continuation of U.S. application Ser. No. 16/482,038, filed Jul. 30, 2019, issued Dec. 21, 2021, U.S. Pat. No. 11,203,636, which is a 35 U.S.C. 371 national stage filing of International Application No. PCT/US2018/016508, filed on Feb. 1, 2018, which claims priority to U.S. Application No. 62/453,257 filed on Feb. 1, 2017. The contents of the aforementioned applications are hereby incorporated by reference in their entireties.

### STATEMENT REGARDING FEDERALLY SPONSORED RESEARCH OR DEVELOPMENT

This invention was made with government support under grants 5R01HL128973 and 4K23HL114868 awarded by the National Institutes of Health. The government has certain rights in the invention.

### STATEMENT REGARDING JOINT RESEARCH AGREEMENT

This invention was made under a Joint Research Agreement by and among Yale University, MedImmune Ltd., AstraZeneca Pharmaceuticals LP, and Corvidia Therapeutics, Inc.

### SEQUENCE LISTING

The instant application contains a Sequence Listing which has been in XML format via the USPTO electronic filing system and is hereby incorporated herein by reference in its entirety. Said XML file, created on Nov. 21, 2025, is named “200109US04_sequence_listing.xml”, and is 29,683 bytes in size.

### BACKGROUND

Renal injury is often observed in heart failure, and heart failure is often observed in renal disease. The term cardiorenal syndrome (“CRS”) encompasses a variety of clinical conditions in which dysfunction of the heart, kidney, or both, leads to accelerated failure of both organs. Evidence of this adverse organ crosstalk portends a high degree of morbidity and mortality. Despite its dire clinical implications, the mechanistic underpinnings of CRS are only now being elucidated.

There is a need for new methods of treating renal injury and renal impairment in heart failure patients. There is also a need for new methods of detecting cardiorenal syndrome, for determining which cardiorenal patients will be responsive to treatment, and for monitoring efficacy of therapy.

Diuretics are a mainstay in the treatment of heart failure. However, certain patients are, or become, resistant to diuretics. There is a need for new methods of treating patients who require diuretics but are resistant to diuretics.

### SUMMARY

As further described below in Example 1, consecutive heart failure (“HF”) patients receiving high dose diuretic therapy at an outpatient treatment center were enrolled in a prospective observational study. Plasma levels of IL-6 were measured to query systemic associations of this pro-inflammatory cytokine with various disease parameters, and urine levels of IL-6 were measured to query IL-6 associations with local inflammation and neurohormonal activation at the level of renal tissues.

Urine IL-6 and plasma IL-6 levels were found to be only modestly correlated with one another.

Increases in urine levels of IL-6 were significantly correlated in these heart failure patients with measures of renal impairment, such as diuretic resistance, lower estimated glomerular filtration rate (“eGFR”), and increased tissue-level renin-angiotensin-aldosterone system (“RAAS”) activation.

Although an inverse association between diuretic efficiency and plasma IL-6 was also observed, upon adjustment for eGFR, only urine IL-6 remained significantly associated with risk of low diuretic efficiency in these patients. Furthermore, when urine IL-6 and plasma IL-6 were both entered into a logistic regression model, only urine IL-6 remained associated with risk of low diuretic efficiency while plasma IL-6 showed no such association.

These data demonstrate that urine IL-6 level is a useful biomarker for renal inflammation, and can be used to gauge renal dysfunction in the setting of heart failure (cardiorenal syndrome). The data further suggest that serial measurements of urine IL-6 can be used to measure the renal benefits of treatments administered to patients with heart failure, notably heart failure patients with cardiorenal syndrome.

The urine IL-6 data, and to some extent the plasma IL-6 data, also predict that treatment with an IL 6 antagonist should be effective to reduce renal inflammation in heart failure patients, that is, to treat renal symptoms of cardiorenal syndrome.

However, because infection is often a precipitating cause of acute decompensation in heart failure patients, it is important to limit anti-cytokine and other immunosuppressive therapies to those heart failure patients who are likely to respond with improved renal and/or cardiac function. The cost of chronic IL-6 antagonist therapy also militates for limiting treatment to those heart failure patients who are likely to respond with improved renal and/or cardiac function.

Analysis was expanded to 129 patients, and further assessed each patient's genotype at the rs855791 single nucleotide polymorphism (“SNP”) in transmembrane protease serine 6 (“TMPRSS6”).

Urine levels of IL-6 were inversely correlated with diuretic efficiency only in the patients having at least one copy of the major allele of the TMPRSS6 rs855791 SNP (AG and GG); urine levels of IL-6 were not significantly correlated with diuretic efficiency in patients homozygous for the minor allele (AA). Plasma levels of IL-6 correlated inversely with diuretic efficiency only in the patients having at least one copy of the major allele of the TMPRSS6 rs855791 SNP; plasma levels of IL-6 were not significantly correlated with diuretic efficiency in patients homozygous for the minor allele.

These data suggested that diuretic resistance (low diuretic efficiency) in heart failure patients could be treated with an IL-6 antagonist, but only in those having at least one copy of the TMPRSS6 rs855791 major allele.

In mouse M1 CCD cells, which are genotypically analogous to human cells homozygous for the TMPRSS6 rs855791 major allele, the addition of IL-6 correlated with the expression of ion transporters, NKCC2, ENaC-beta, and NCC. Increased expression of these ion transporters provides a putative mechanism for IL-6 mediated diuretic resistance.

Because the IL-6 mediated increase in expression of ion transporters is not known to be linked to hepcidin expression, these data suggested that IL-6 antagonism could also be effective in treating diuretic resistance in patients homozygous for the TMPRSS6 rs855791 minor allele.

Secondary analysis of data from two additional large heart failure clinical trials confirmed the association of diuretic resistance with IL-6 level (Example 5), independently of TMPRSS6 rs855791 genotype (Example 6), providing evidence that IL-6 antagonism should also be effective in treating diuretic resistance in patients homozygous for the TMPRSS6 rs855791 minor allele.

Accordingly, in a first aspect, methods are provided for treating a patient who requires diuresis but is resistant to diuretics. The methods comprise administering, in combination with a diuretic, a therapeutically effective amount of an IL-6 antagonist to the patient.

In some embodiments, the patient has elevated pre-treatment plasma IL-6 levels. In certain embodiments, the patient has a pre-treatment plasma IL-6 level of greater than 2 pg/mL. In certain embodiments, the patient has a pre-treatment IL-6 level of greater than 3 pg/mL. In certain embodiments, the patient has a pre-treatment IL-6 level of greater than 5 pg/mL. In certain embodiments, the patient has a pre-treatment IL-6 level of greater than 10 pg/mL.

In some embodiments, the patient has a diuretic efficiency of less than 500. In some embodiments, the patient has a diuretic efficiency of less than 200. In some embodiments, the patient has a diuretic efficiency of less than 150. In some embodiments, the patient has a diuretic efficiency of less than 100.

In some embodiments, the patient has diuretic resistant heart failure. In certain embodiments, the patient has acute heart failure. In certain embodiments, the patient has chronic heart failure.

In some embodiments, the patient has cardiorenal syndrome. In some of these embodiments, the patient has cardiorenal syndrome type 4.

In some embodiments, the patient has kidney disease. In certain embodiments, the patient has hepatorenal syndrome.

In some embodiments, the patient has at least one copy of the TMPRSS6 rs855791 major allele.

In certain embodiments, the IL-6 antagonist is an anti-IL-6 antibody, or antigen-binding fragment or derivative thereof. In particular embodiments, the anti-IL-6 antibody or antigen-binding fragment or derivative has a K_{D} for binding human IL-6 of less than 100 nM, less than 50 nM, less than 10 nM, even less than 1 nM.

In certain embodiments, the anti-IL-6 antibody or antigen-binding fragment or derivative has an elimination half-life following intravenous administration of at least 7 days, at least 14 days, at least 21 days, or at least 30 days.

In certain embodiments, the IL-6 antagonist is a full-length monoclonal anti-IL-6 antibody. In particular embodiments, the antibody is an IgG1 or IgG4 antibody. In certain embodiments, the antibody is an IgG1 antibody.

In certain embodiments, the anti-IL-6 antibody or antigen-binding fragment or derivative is fully human. In certain embodiments, the anti-IL-6 antibody or antigen-binding fragment or derivative is humanized.

In certain embodiments, the anti-IL-6 antibody or antigen-binding fragment or derivative comprises all six variable region CDRs of MED5117. In specific embodiments, the antibody comprises the VH and VL of MED5117. In particular embodiments, the antibody is MED5117.

In certain embodiments, the anti-IL-6 antibody or antigen-binding fragment or derivative comprises all six variable region CDRs of an antibody selected from the group consisting of siltuximab, gerilimzumab, sirukumab, clazakizumab, olokizumab, elsilimomab, VX30 (VOP-R003; Vaccinex), EB-007 (EBI-029; Eleven Bio), ARGX-109 (ArGEN-X), FM101 (Femta Pharmaceuticals, Lonza) and ALD518/BMS-945429 (Alder Biopharmaceuticals, Bristol-Myers Squibb). In certain embodiments, the anti-IL-6 antibody or antigen-binding fragment or derivative comprises the heavy chain V region and light chain V region from an antibody selected from the group consisting of siltuximab, gerilimzumab, sirukumab, clazakizumab, olokizumab, VX30 (VOP-R003; Vaccinex), EB-007 (EBI-029; Eleven Bio), ARGX-109 (ArGEN-X), FM101 (Femta Pharmaceuticals, Lonza) and ALD518/BMS-945429 (Alder Biopharmaceuticals, Bristol-Myers Squibb). In particular embodiments, the anti-IL-6 antibody or antigen-binding fragment or derivative is an antibody selected from the group consisting of siltuximab, gerilimzumab, sirukumab, clazakizumab, olokizumab, VX30 (VOP-R003; Vaccinex), EB-007 (EBI-029; Eleven Bio), ARGX-109 (ArGEN-X), FM101 (Femta Pharmaceuticals, Lonza) and ALD518/BMS-945429 (Alder Biopharmaceuticals, Bristol-Myers Squibb).

In certain embodiments, the IL-6 antagonist is a single domain antibody, a V_{HH} Nanobody, an Fab, or a scFv.

In certain embodiments, the IL-6 antagonist is an anti-IL-6R antibody, or antigen-binding fragment or derivative thereof. In certain embodiments, the anti-IL-6R antibody, antigen-binding fragment, or derivative comprises the 6 CDRs of tocilizumab or vobarilizumab.

In certain embodiments, the IL-6 antagonist is a JAK inhibitor. In certain embodiments, the JAK inhibitor is selected from the group consisting of tofacitinib (Xeljanz), decernotinib, ruxolitinib, upadacitinib, baricitinib, filgotinib, lestaurtinib, pacritinib, peficitinib, INCB-039110, ABT-494, INCB-047986 and AC-410.

In certain embodiments, the IL-6 antagonist is a STAT3 inhibitor.

In certain embodiments, the IL-6 antagonist is administered parenterally. In particular embodiments, the IL-6 antagonist is administered subcutaneously.

In certain embodiments, the IL-6 antagonist is administered orally.

In certain embodiments, the IL-6 antagonist is administered at a dose, on a schedule, and for a period sufficient to increase diuretic efficiency. In certain embodiments, the IL-6 antagonist is administered at a dose, on a schedule, and for a period sufficient to increase diuretic efficiency to normal levels. In certain embodiments, the IL-6 antagonist is administered at a dose, on a schedule, and for a period sufficient to increase eGFR. In particular embodiments, the IL-6 antagonist is administered at a dose, on a schedule, and for a period sufficient to increase eGFR to normal levels.

In certain embodiments, the method further comprises the subsequent step of determining the level of IL-6 in urine, determining the level of IL-6 in plasma, or determining the level of IL-6 in urine and in plasma. In particular embodiments, the method further comprises a final step of adjusting the dose of IL-6 antagonist for subsequent administration based on IL-6 level determined in the immediately preceding step.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1A is a bar graph showing diuretic efficiency (“DE”) by tertiles of urine IL-6 in 129 heart failure (“HF”) patients receiving high dose diuretic therapy who were enrolled in the study described in Example 1 herein. Whiskers extend from the 10^{th} to 90^{th} percentile.

FIG. 1B is a bar graph showing diuretic efficiency (“DE”) by tertiles of plasma IL-6 in the 129 heart failure (“HF”) patients whose data is shown in FIG. 1A. Whiskers extend from the 10^{th} to 90^{th} percentile.

FIG. 2A plots DE by tertiles of urine IL-6 in the patients reported in FIGS. 1A and 1B further stratified by genotype, with the left panel showing results for patients homozygous for the TMPRSS6 rs855791 SNP minor allele (2321G→A; A736V) and right panel showing results for patients having at least one copy of the TMPRSS6 rs855791 SNP major allele (2321G; A736).

FIG. 2B plots DE by tertiles of plasma IL-6 in the patients reported in FIGS. 1A and 1B further stratified by genotype, with the left panel showing results for patients homozygous for the TMPRSS6 rs855791 SNP minor allele (2321G→A; A736V) and right panel showing results for patients having at least one copy of the TMPRSS6 rs855791 SNP major allele (2321G; A736).

FIGS. 3A and 3B plot the association in a 98 patient subset of the 129 patients reported in FIGS. 1 and 2 of (A) Urine IL-6 levels and (B) Plasma IL-6 levels with the following clinical determinations: Reduced kidney function, low diuretic efficiency (“DE”), increased neurohormonal activation, and risk of mortality. Whiskers represent 95% confidence interval (“CI”). All analyses adjusted for both urine and plasma levels of IL-6. Urine IL-6 levels are indexed to urinary creatinine. Due to the skewed distribution of urine and plasma IL-6 variables, a log transform was applied before performing logistic and Cox regressions. Abbreviations: OR=Odds ratio. HR=Hazards ratio. IL=interleukin. SD=standard deviation. eGFR=estimated glomerular filtration rate. *=adjusted for use of angiotensin converting enzyme inhibitor (ACE-I) or angiotensin receptor blocker (ARB). **=adjusted for baseline characteristics including age, race, amino terminal pro B-type natriuretic peptide (NT-proBNP), use of ACE-I or ARB, home loop diuretic dose, and eGFR.

FIGS. 4A, 4B, and 4C show the expression of NKCC2, ENaC-beta, and NCC in M1 CCD cells after treatment with IL-6 and/or Ruxolitinib, with FIG. 4A showing the expression of NKCC2 after the treatment with IL-6 and/or Ruxolitinib; FIG. 4B showing the expression of ENaC-beta after the treatment with IL-6 and/or Ruxolitinib; and FIG. 4C showing the expression of NCC after the treatment with IL-6 and/or Ruxolitinib.

FIGS. 5A, 5B, and 5C show the expression of NKCC2, ENaC-beta, and NCC in M1 CCD cells after treatment with IL-6 and/or Spironolactone, with FIG. 5A showing the expression of NKCC2 after the treatment with IL-6 and/or Spironolactone; FIG. 5B showing the expression of ENaC-beta after the treatment with IL-6 and/or Spironolactone; and FIG. 5C showing the expression of NCC after the treatment with IL-6 and/or Spironolactone.

FIG. 6 shows the association of baseline characteristics with higher levels of IL-6 in the PROTECT trial described in Example 5.

FIGS. 7A and 7B show the Kaplan-Meier survival curve by tertiles of IL-6, with FIG. 7A showing the all-cause mortality at 180 days and FIG. 7B showing all-cause mortality or cardiovascular related rehospitalization at 60 days.

FIGS. 8A and 8B show the Kaplan-Meier survival curve for change of IL-6 between baseline and day 7, with FIG. 8A showing the all-cause mortality at 180 days and FIG. 8B showing all-cause mortality or cardiovascular related rehospitalization at 60 days.

FIG. 9 shows the association of baseline characteristics with higher levels of IL-6 in the BIOSTAT-CHF study described in Example 6.

FIGS. 10A and 10B show the Kaplan-Meier survival curve by tertiles of IL-6, with FIG. 10A showing the all-cause mortality and/or rehospitalization for heart failure at 2 years and FIG. 10B showing all-cause mortality at 2 years.

FIG. 11 show the analysis between ferritin levels and tertiles of IL-6.

### DETAILED DESCRIPTION

### 8.1. Definitions

Unless defined otherwise, all technical and scientific terms used herein have the same meaning as commonly understood by one of ordinary skill in the art to which the invention pertains. It is also to be understood that the terminology used herein is for the purpose of describing particular embodiments only, and is not intended to be limiting.

By “transmembrane protease serine 6 (TMPRSS6) polypeptide” is meant a polypeptide or fragment thereof having at least about 85% or greater amino acid identity to the amino acid sequence provided at NCBI Accession No. NP_001275929 and having serine proteinase activity. The TMPRSS6 polypeptide, also known as Matriptase-2 (MT2), cleaves hemojuvelin and inhibits bone morphogenetic protein signaling. An exemplary TMPRSS6 amino acid sequence having an alanine at position 736 (736A) is provided below:

| (SEQ ID NO: 1) | |
|---|---|
| 1 | MPVAEAPQVA GGQGDGGDGE EAEPEGMFKA CEDSKRKARG YLRLVPLFVL LALLVLASAG |
| 61 | VLLWYFLGYK AEVMVSQVYS GSLRVLNRHF SQDLTRRESS AFRSETAKAQ KMLKELITST |
| 121 | RLGTYYNSSS VYSFGEGPLT CFFWFILQIP EHRRLMLSPE VVQALLVEEL LSTVNSSAAV |
| 181 | PYRAEYEVDP EGLVILEASV KDIAALNSTL GCYRYSYVGQ GQVLRLKGPD HLASSCLWHL |
| 241 | QGPKDLMLKL RLEWTLAECR DRLAMYDVAG PLEKRLITSV YGCSRQEPVV EVLASGAIMA |
| 301 | VVWKKGLHSY YDPFVLSVQP VVFQACEVNL TLDNRLDSQG VLSTPYFPSY YSPQTHCSWH |
| 361 | LTVPSLDYGL ALWFDAYALR RQKYDLPCTQ GQWTIQNRRL CGLRILQPYA ERIPVVATAG |
| 421 | ITINFTSQIS LTGPGVRVHY GLYNQSDPCP GEFLCSVNGL CVPACDGVKD CPNGLDERNC |
| 481 | VCRATFQCKE DSTCISLPKV CDGQPDCLNG SDEEQCQEGV PCGTFTFQCE DRSCVKKPNP |
| 541 | QCDGRPDCRD GSDEEHCDCG LQGPSSRIVG GAVSSEGEWP WQASLQVRGR HICGGALIAD |
| 601 | RWVITAAHCF QEDSMASTVL WTVFLGKVWQ NSRWPGEVSF KVSRLLLHPY HEEDSHDYDV |
| 661 | ALLQLDHPVV RSAAVRPVCL PARSHFFEPG LHCWITGWGA LREGALRADA VALFYGWRNQ |
| 721 | GSETCCCPIS NALQKADVQL IPQDLCSEVY RYQVTPRMLC AGYRKGKKDA CQGDSGGPLV |
| 781 | CKALSGRWFL AGLVSWGLGC GRPNYFGVYT RITGVISWIQ QVVT |

An exemplary TMPRSS6 amino acid sequence having a valine at position 736 (736V) is provided below:

| (SEQ ID NO: 2) | |
|---|---|
| 1 | MPVAEAPQVA GGQGDGGDGE EAEPEGMFKA CEDSKRKARG YLRLVPLFVL LALLVLASAG |
| 61 | VLLWYFLGYK AEVMVSQVYS GSLRVLNRHF SQDLTRRESS AFRSETAKAQ KMLKELITST |
| 121 | RLGTYYNSSS VYSFGEGPLT CFFWFILQIP EHRRLMLSPE VVQALLVEEL LSTVNSSAAV |
| 181 | PYRAEYEVDP EGLVILEASV KDIAALNSTL GCYRYSYVGQ GQVLRLKGPD HLASSCLWHL |
| 24 | QGPKDLMLKL RLEWTLAECR DRLAMYDVAG PLEKRLITSV YGCSRQEPVV EVLASGAIMA |
| 30 | VVWKKGLHSY YDPFVLSVQP VVFQACEVNL TLDNRLDSQG VLSTPYFPSY YSPQTHCSWH |
| 36 | LTVPSLDYGL ALWFDAYALR RQKYDLPCTQ GQWTIQNRRL CGLRILQPYA ERIPVVATAG |
| 42 | ITINFTSQIS LTGPGVRVHY GLYNQSDPCP GEFLCSVNGL CVPACDGVKD CPNGLDERNC |
| 48 | VCRATFQCKE DSTCISLPKV CDGQPDCLNG SDEEQCQEGV PCGTFTFQCE DRSCVKKPNP |
| 54 | QCDGRPDCRD GSDEEHCDCG LQGPSSRIVG GAVSSEGEWP WQASLQVRGR HICGGALIAD |
| 60 | RWVITAAHCF QEDSMASTVL WTVFLGKVWQ NSRWPGEVSF KVSRLLLHPY HEEDSHDYDV |
| 66 | ALLQLDHPVV RSAAVRPVCL PARSHFFEPG LHCWITGWGA LREGALRADA VALFYGWRNQ |
| 72 | GSETCCCPIS NALQKVDVQL IPQDLCSEVY RYQVTPRMLC AGYRKGKKDA CQGDSGGPLV |
| 78 | CKALSGRWFL AGLVSWGLGC GRPNYFGVYT RITGVISWIQ QVVT |

By “TMPRSS6 nucleic acid molecule” is meant a polynucleotide encoding a TMPRSS6 polypeptide (Matriptase-2; MT2). An exemplary TMPRSS6 nucleic acid molecule sequence is provided at NCBI Accession No. NM_001289000. A TMPRSS6 nucleic acid sequence having a G at nucleotide position 2321 (“G allele”; “major allele”) is provided below:

| (SEQ ID NO: 3) | |
|---|---|
| 1 | GGACAAACAG AGGCTCCTGA GGCCTGTGTG CAGGCCCGGC ACCTATCTGC CGCTCCCAAA |
| 61 | GGATGCCCGT GGCCGAGGCC CCCCAGGTGG CTGGCGGGCA GGGGGACGGA GGTGATGGCG |
| 121 | AGGAAGCGGA GCCGGAGGGG ATGTTCAAGG CCTGTGAGGA CTCCAAGAGA AAAGCCCGGG |
| 181 | GCTACCTCCG CCTGGTGCCC CTGTTTGTGC TGCTGGCCCT GCTCGTGCTG GCTTCGGCGG |
| 241 | GGGTGCTACT CTGGTATTTC CTAGGGTACA AGGCGGAGGT GATGGTCAGC CAGGTGTACT |
| 301 | CAGGCAGTCT GCGTGTACTC AATCGCCACT TCTCCCAGGA TCTTACCCGC CGGGAATCTA |
| 361 | GTGCCTTCCG CAGTGAAACC GCCAAAGCCC AGAAGATGCT CAAGGAGCTC ATCACCAGCA |
| 421 | CCCGCCTGGG AACTTACTAC AACTCCAGCT CCGTCTATTC CTTTGGGGAG GGACCCCTCA |
| 481 | CCTGCTTCTT CTGGTTCATT CTCCAAATCC CCGAGCACCG CCGGCTGATG CTGAGCCCCG |
| 541 | AGGTGGTGCA GGCACTGCTG GTGGAGGAGC TGCTGTCCAC AGTCAACAGC TCGGCTGCCG |
| 601 | TCCCCTACAG GGCCGAGTAC GAAGTGGACC CCGAGGGCCT AGTGATCCTG GAAGCCAGTG |
| 661 | TGAAAGACAT AGCTGCATTG AATTCCACGC TGGGTTGTTA CCGCTACAGC TACGTGGGCC |
| 721 | AGGGCCAGGT CCTCCGGCTG AAGGGGCCTG ACCACCTGGC CTCCAGCTGC CTGTGGCACC |
| 781 | TGCAGGGCCC CAAGGACCTC ATGCTCAAAC TCCGGCTGGA GTGGACGCTG GCAGAGTGCC |
| 841 | GGGACCGACT GGCCATGTAT GACGTGGCCG GGCCCCTGGA GAAGAGGCTC ATCACCTCGG |
| 901 | TGTACGGCTG CAGCCGCCAG GAGCCCGTGG TGGAGGTTCT GGCGTCGGGG GCCATCATGG |
| 961 | CGGTCGTCTG GAAGAAGGGC CTGCACAGCT ACTACGACCC CTTCGTGCTC TCCGTGCAGC |
| 1021 | CGGTGGTCTT CCAGGCCTGT GAAGTGAACC TGACGCTGGA CAACAGGCTC GACTCCCAGG |
| 1081 | GCGTCCTCAG CACCCCGTAC TTCCCCAGCT ACTACTCGCC CCAAACCCAC TGCTCCTGGC |
| 1141 | ACCTCACGGT GCCCTCTCTG GACTACGGCT TGGCCCTCTG GTTTGATGCC TATGCACTGA |
| 1201 | GGAGGCAGAA GTATGATTTG CCGTGCACCC AGGGCCAGTG GACGATCCAG AACAGGAGGC |
| 1261 | TGTGTGGCTT GCGCATCCTG CAGCCCTACG CCGAGAGGAT CCCCGTGGTG GCCACGGCCG |
| 1321 | GGATCACCAT CAACTTCACC TCCCAGATCT CCCTCACCGG GCCCGGTGTG CGGGTGCACT |
| 1381 | ATGGCTTGTA CAACCAGTCG GACCCCTGCC CTGGAGAGTT CCTCTGTTCT GTGAATGGAC |
| 1441 | TCTGTGTCCC TGCCTGTGAT GGGGTCAAGG ACTGCCCCAA CGGCCTGGAT GAGAGAAACT |
| 1501 | GCGTTTGCAG AGCCACATTC CAGTGCAAAG AGGACAGCAC ATGCATCTCA CTGCCCAAGG |
| 1561 | TCTGTGATGG GCAGCCTGAT TGTCTCAACG GCAGCGACGA AGAGCAGTGC CAGGAAGGGG |
| 1621 | TGCCATGTGG GACATTCACC TTCCAGTGTG AGGACCGGAG CTGCGTGAAG AAGCCCAACC |
| 1681 | CGCAGTGTGA TGGGCGGCCC GACTGCAGGG ACGGCTCGGA TGAGGAGCAC TGTGACTGTG |
| 1741 | GCCTCCAGGG CCCCTCCAGC CGCATTGTTG GTGGAGCTGT GTCCTCCGAG GGTGAGTGGC |
| 1801 | CATGGCAGGC CAGCCTCCAG GTTCGGGGTC GACACATCTG TGGGGGGGCC CTCATCGCTG |
| 1861 | ACCGCTGGGT GATAACAGCT GCCCACTGCT TCCAGGAGGA CAGCATGGCC TCCACGGTGC |
| 1921 | TGTGGACCGT GTTCCTGGGC AAGGTGTGGC AGAACTCGCG CTGGCCTGGA GAGGTGTCCT |
| 1981 | TCAAGGTGAG CCGCCTGCTC CTGCACCCGT ACCACGAAGA GGACAGCCAT GACTACGACG |
| 2041 | TGGCGCTGCT GCAGCTCGAC CACCCGGTGG TGCGCTCGGC CGCCGTGCGC CCCGTCTGCC |
| 2101 | TGCCCGCGCG CTCCCACTTC TTCGAGCCCG GCCTGCACTG CTGGATTACG GGCTGGGGCG |
| 2161 | CCTTGCGCGA GGGCGCCCTA CGGGCGGATG CTGTGGCCCT ATTTTATGGA TGGAGAAACC |
| 2221 | AAGGCTCAGA GACATGTTGC TGCCCCATCA GCAACGCTCT GCAGAAAGTG GATGTGCAGT |
| 2281 | TGATCCCACA GGACCTGTGC AGCGAGGTCT ATCGCTACCA GGTGACGCCA CGCATGCTGT |
| 2341 | GTGCCGGCTA CCGCAAGGGC AAGAAGGATG CCTGTCAGGG TGACTCAGGT GGTCCGCTGG |
| 2401 | TGTGCAAGGC ACTCAGTGGC CGCTGGTTCC TGGCGGGGCT GGTCAGCTGG GGCCTGGGCT |
| 2461 | GTGGCCGGCC TAACTACTTC GGCGTCTACA CCCGCATCAC AGGTGTGATC AGCTGGATCC |
| 2521 | AGCAAGTGGT GACCTGAGGA ACTGCCCCCC TGCAAAGCAG GGCCCACCTC CTGGACTCAG |
| 2581 | AGAGCCCAGG GCAACTGCCA AGCAGGGGGA CAAGTATTCT GGCGGGGGGT GGGGGAGAGA |
| 2641 | GCAGGCCCTG TGGTGGCAGG AGGTGGCATC TTGTCTCGTC CCTGATGTCT GCTCCAGTGA |
| 2701 | TGGCAGGAGG ATGGAGAAGT GCCAGCAGCT GGGGGTCAAG ACGTCCCCTG AGGACCCAGG |
| 2761 | CCCACACCCA GCCCTTCTGC CTCCCAATTC TCTCTCCTCC GTCCCCTTCC TCCACTGCTG |
| 2821 | CCTAATGCAA GGCAGTGGCT CAGCAGCAAG AATGCTGGTT CTACATCCCG AGGAGTGTCT |
| 2881 | GAGGTGCGCC CCACTCTGTA CAGAGGCTGT TTGGGCAGCC TTGCCTCCAG AGAGCAGATT |
| 2941 | CCAGCTTCGG AAGCCCCTGG TCTAACTTGG GATCTGGGAA TGGAAGGTGC TCCCATCGGA |
| 3001 | GGGGACCCTC AGAGCCCTGG AGACTGCCAG GTGGGCCTGC TGCCACTGTA AGCCAAAAGG |
| 3061 | TGGGGAAGTC CTGACTCCAG GGTCCTTGCC CCACCCCTGC CTGCCACCTG GGCCCTCACA |
| 3121 | GCCCAGACCC TCACTGGGAG GTGAGCTCAG CTGCCCTTTG GAATAAAGCT GCCTGATCCA |
| 3181 | AAAAAAAAAA AAAAAA |

A TMPRSS6 nucleic acid sequence having an A at nucleotide position 2321 is provided below:

| (SEQ ID NO: 4) | |
|---|---|
| 1 | GGACAAACAG AGGCTCCTGA GGCCTGTGTG CAGGCCCGGC ACCTATCTGC CGCTCCCAAA |
| 61 | GGATGCCCGT GGCCGAGGCC CCCCAGGTGG CTGGCGGGCA GGGGGACGGA GGTGATGGCG |
| 121 | AGGAAGCGGA GCCGGAGGGG ATGTTCAAGG CCTGTGAGGA CTCCAAGAGA AAAGCCCGGG |
| 181 | GCTACCTCCG CCTGGTGCCC CTGTTTGTGC TGCTGGCCCT GCTCGTGCTG GCTTCGGCGG |
| 241 | GGGTGCTACT CTGGTATTTC CTAGGGTACA AGGCGGAGGT GATGGTCAGC CAGGTGTACT |
| 301 | CAGGCAGTCT GCGTGTACTC AATCGCCACT TCTCCCAGGA TCTTACCCGC CGGGAATCTA |
| 361 | GTGCCTTCCG CAGTGAAACC GCCAAAGCCC AGAAGATGCT CAAGGAGCTC ATCACCAGCA |
| 421 | CCCGCCTGGG AACTTACTAC AACTCCAGCT CCGTCTATTC CTTTGGGGAG GGACCCCTCA |
| 481 | CCTGCTTCTT CTGGTTCATT CTCCAAATCC CCGAGCACCG CCGGCTGATG CTGAGCCCCG |
| 541 | AGGTGGTGCA GGCACTGCTG GTGGAGGAGC TGCTGTCCAC AGTCAACAGC TCGGCTGCCG |
| 601 | TCCCCTACAG GGCCGAGTAC GAAGTGGACC CCGAGGGCCT AGTGATCCTG GAAGCCAGTG |
| 661 | TGAAAGACAT AGCTGCATTG AATTCCACGC TGGGTTGTTA CCGCTACAGC TACGTGGGCC |
| 721 | AGGGCCAGGT CCTCCGGCTG AAGGGGCCTG ACCACCTGGC CTCCAGCTGC CTGTGGCACC |
| 781 | TGCAGGGCCC CAAGGACCTC ATGCTCAAAC TCCGGCTGGA GTGGACGCTG GCAGAGTGCC |
| 841 | GGGACCGACT GGCCATGTAT GACGTGGCCG GGCCCCTGGA GAAGAGGCTC ATCACCTCGG |
| 901 | TGTACGGCTG CAGCCGCCAG GAGCCCGTGG TGGAGGTTCT GGCGTCGGGG GCCATCATGG |
| 961 | CGGTCGTCTG GAAGAAGGGC CTGCACAGCT ACTACGACCC CTTCGTGCTC TCCGTGCAGC |
| 1021 | CGGTGGTCTT CCAGGCCTGT GAAGTGAACC TGACGCTGGA CAACAGGCTC GACTCCCAGG |
| 1081 | GCGTCCTCAG CACCCCGTAC TTCCCCAGCT ACTACTCGCC CCAAACCCAC TGCTCCTGGC |
| 1141 | ACCTCACGGT GCCCTCTCTG GACTACGGCT TGGCCCTCTG GTTTGATGCC TATGCACTGA |
| 1201 | GGAGGCAGAA GTATGATTTG CCGTGCACCC AGGGCCAGTG GACGATCCAG AACAGGAGGC |
| 1261 | TGTGTGGCTT GCGCATCCTG CAGCCCTACG CCGAGAGGAT CCCCGTGGTG GCCACGGCCG |
| 1321 | GGATCACCAT CAACTTCACC TCCCAGATCT CCCTCACCGG GCCCGGTGTG CGGGTGCACT |
| 1381 | ATGGCTTGTA CAACCAGTCG GACCCCTGCC CTGGAGAGTT CCTCTGTTCT GTGAATGGAC |
| 1441 | TCTGTGTCCC TGCCTGTGAT GGGGTCAAGG ACTGCCCCAA CGGCCTGGAT GAGAGAAACT |
| 1501 | GCGTTTGCAG AGCCACATTC CAGTGCAAAG AGGACAGCAC ATGCATCTCA CTGCCCAAGG |
| 1561 | TCTGTGATGG GCAGCCTGAT TGTCTCAACG GCAGCGACGA AGAGCAGTGC CAGGAAGGGG |
| 1621 | TGCCATGTGG GACATTCACC TTCCAGTGTG AGGACCGGAG CTGCGTGAAG AAGCCCAACC |
| 1681 | CGCAGTGTGA TGGGCGGCCC GACTGCAGGG ACGGCTCGGA TGAGGAGCAC TGTGACTGTG |
| 1741 | GCCTCCAGGG CCCCTCCAGC CGCATTGTTG GTGGAGCTGT GTCCTCCGAG GGTGAGTGGC |
| 1801 | CATGGCAGGC CAGCCTCCAG GTTCGGGGTC GACACATCTG TGGGGGGGCC CTCATCGCTG |
| 1861 | ACCGCTGGGT GATAACAGCT GCCCACTGCT TCCAGGAGGA CAGCATGGCC TCCACGGTGC |
| 1921 | TGTGGACCGT GTTCCTGGGC AAGGTGTGGC AGAACTCGCG CTGGCCTGGA GAGGTGTCCT |
| 1981 | TCAAGGTGAG CCGCCTGCTC CTGCACCCGT ACCACGAAGA GGACAGCCAT GACTACGACG |
| 2041 | TGGCGCTGCT GCAGCTCGAC CACCCGGTGG TGCGCTCGGC CGCCGTGCGC CCCGTCTGCC |
| 2101 | TGCCCGCGCG CTCCCACTTC TTCGAGCCCG GCCTGCACTG CTGGATTACG GGCTGGGGCG |
| 2161 | CCTTGCGCGA GGGCGCCCTA CGGGCGGATG CTGTGGCCCT ATTTTATGGA TGGAGAAACC |
| 2221 | AAGGCTCAGA GACATGTTGC TGCCCCATCA GCAACGCTCT GCAGAAAGTG GATGTGCAGT |
| 2281 | TGATCCCACA GGACCTGTGC AGCGAGGTCT ATCGCTACCA AGTGACGCCA CGCATGCTGT |
| 2341 | GTGCCGGCTA CCGCAAGGGC AAGAAGGATG CCTGTCAGGG TGACTCAGGT GGTCCGCTGG |
| 2401 | TGTGCAAGGC ACTCAGTGGC CGCTGGTTCC TGGCGGGGCT GGTCAGCTGG GGCCTGGGCT |
| 2461 | GTGGCCGGCC TAACTACTTC GGCGTCTACA CCCGCATCAC AGGTGTGATC AGCTGGATCC |
| 2521 | AGCAAGTGGT GACCTGAGGA ACTGCCCCCC TGCAAAGCAG GGCCCACCTC CTGGACTCAG |
| 2581 | AGAGCCCAGG GCAACTGCCA AGCAGGGGGA CAAGTATTCT GGCGGGGGGT GGGGGAGAGA |
| 2641 | GCAGGCCCTG TGGTGGCAGG AGGTGGCATC TTGTCTCGTC CCTGATGTCT GCTCCAGTGA |
| 2701 | TGGCAGGAGG ATGGAGAAGT GCCAGCAGCT GGGGGTCAAG ACGTCCCCTG AGGACCCAGG |
| 2761 | CCCACACCCA GCCCTTCTGC CTCCCAATTC TCTCTCCTCC GTCCCCTTCC TCCACTGCTG |
| 2821 | CCTAATGCAA GGCAGTGGCT CAGCAGCAAG AATGCTGGTT CTACATCCCG AGGAGTGTCT |
| 2881 | GAGGTGCGCC CCACTCTGTA CAGAGGCTGT TTGGGCAGCC TTGCCTCCAG AGAGCAGATT |
| 2941 | CCAGCTTCGG AAGCCCCTGG TCTAACTTGG GATCTGGGAA TGGAAGGTGC TCCCATCGGA |
| 3001 | GGGGACCCTC AGAGCCCTGG AGACTGCCAG GTGGGCCTGC TGCCACTGTA AGCCAAAAGG |
| 3061 | TGGGGAAGTC CTGACTCCAG GGTCCTTGCC CCACCCCTGC CTGCCACCTG GGCCCTCACA |
| 3121 | GCCCAGACCC TCACTGGGAG GTGAGCTCAG CTGCCCTTTG GAATAAAGCT GCCTGATCCA |
| 3181 | AAAAAAAAAA AAAAAA |

By “variant” is meant a polynucleotide or polypeptide sequence that differs from a reference sequence by one or more nucleotides or one or more amino acids. An exemplary TMPRSS6 variant is TMPRSS6 (A736V), resulting from SNP rs855791 (G→A).

By “single nucleotide polymorphism” or “SNP” is meant a naturally occurring DNA sequence variant in which a single nucleotide in the genome differs between members of a biological species or between paired chromosomes in an individual. SNPs can be used as genetic markers for variant alleles. In one embodiment, the TMPRSS6 SNP is rs855791.

By “rs855791” is meant a single nucleotide polymorphism (SNP) in the human TMPRSS6 gene, 2321G→A, resulting in an alanine to valine substitution (A736V) in the catalytic domain of Matriptase-2 (MT2), which is encoded by the TMPRSS6 gene. The allele with highest frequency in the human population (the major allele) is 2321G, encoding 736A. The allele with lowest frequency in the human population (minor allele) is 2321A, encoding 736V.

By “heterozygous” is meant that a chromosomal locus has two different alleles. In one embodiment of the methods described herein, heterozygous refers to a genotype in which one allele has a TMPRSS6 nucleic acid sequence encoding a TMPRSS6 polypeptide having an alanine at amino acid position 736 (e.g., having a G or C at nucleotide position 2321 of a TMPRSS6 nucleic acid molecule) (rs855791 major allele), and the other allele has a variant TMPRSS6 nucleic acid sequence encoding a TMPRSS6 polypeptide comprising a valine at amino acid position 736 (e.g., having an A or T at nucleotide position 2321 of a TMPRSS6 nucleic acid molecule) (rs855791 minor allele).

By “homozygous” is meant that a chromosomal locus has two identical alleles. In certain embodiments of the methods described herein, homozygous refers to a genotype in which both alleles have a TMPRSS6 nucleic acid sequence encoding a TMPRSS6 polypeptide comprising an alanine at amino acid position 736 (e.g., having a G or C at nucleotide position 2321 of a TMPRSS6 nucleic acid molecule) (rs855791 homozygous major allele). In certain embodiments, homozygous refers to a genotype in which both alleles have a TMPRSS6 nucleic acid sequence encoding a TMPRSS6 polypeptide comprising a valine at amino acid position 736 (e.g., having an A or T at nucleotide position 2321 of a TMPRSS6 nucleic acid molecule) (rs855791 homozygous minor allele).

“Determining that a patient has at least one copy of the TMPRSS6 rs855791 major allele” includes, but is not limited to, performing an assay to determine that a patient has at least one copy of the TMPRSS6 rs855791 major allele; ordering an assay to determine that a patient has at least one copy of the TMPRSS6 rs855791 major allele; prescribing an assay to determine that a patient has at least one copy of the TMPRSS6 rs855791 major allele; otherwise directing or controlling that an assay be performed to determine that a patient has at least one copy of the TMPRSS6 rs855791 major allele; and reviewing TMRSS6 genotype assay data or protein or nucleic acid sequence data to determine that a patient has at least one copy of the TMPRSS6 rs855791 major allele.

By “interleukin 6” or “IL-6” or “IL-6 polypeptide” is meant a polypeptide or fragment thereof having at least about 85% or greater amino acid identity to the amino acid sequence provided at NCBI Accession No. NP_000591 and having IL-6 biological activity. IL-6 is a pleotropic cytokine with multiple biologic functions. Exemplary IL-6 biological activities include immunostimulatory and pro-inflammatory activities. An exemplary IL-6 amino acid sequence is provided below:

| (SEQ ID NO: 5) | |
|---|---|
| 1 | MCVGARRLGR GPCAALLLLG LGLSTVTGLH CVGDTYPSND RCCHECRPGN GMVSRCSRSQ |
| 61 | NTVCRPCGPG FYNDVVSSKP CKPCTWCNLR SGSERKQLCT ATQDTVCRCR AGTQPLDSYK |
| 121 | PGVDCAPCPP GHFSPGDNQA CKPWTNCTLA GKHTLQPASN SSDAICEDRD PPATQPQETQ |
| 181 | GPPARPITVQ PTEAWPRTSQ GPSTRPVEVP GGRAVAAILG LGLVLGLLGP LAILLALYLL |
| 241 | RRDQRLPPDA HKPPGGGSFR TPIQEEQADA HSTLAKI |

By “Interleukin 6 (IL-6) nucleic acid” is meant a polynucleotide encoding an interleukin 6 (IL-6) polypeptide. An exemplary interleukin 6 (IL-6) nucleic acid sequence is provided at NCBI Accession No. NM_000600. The exemplary sequence at NCBI Accession No. NM_000600 is provided below.

| (SEQ ID NO: 6) | |
|---|---|
| 1 | AATATTAGAG TCTCAACCCC CAATAAATAT AGGACTGGAG ATGTCTGAGG CTCATTCTGC |
| 61 | CCTCGAGCCC ACCGGGAACG AAAGAGAAGC TCTATCTCCC CTCCAGGAGC CCAGCTATGA |
| 121 | ACTCCTTCTC CACAAGCGCC TTCGGTCCAG TTGCCTTCTC CCTGGGGCTG CTCCTGGTGT |
| 181 | TGCCTGCTGC CTTCCCTGCC CCAGTACCCC CAGGAGAAGA TTCCAAAGAT GTAGCCGCCC |
| 241 | CACACAGACA GCCACTCACC TCTTCAGAAC GAATTGACAA ACAAATTCGG TACATCCTCG |
| 301 | ACGGCATCTC AGCCCTGAGA AAGGAGACAT GTAACAAGAG TAACATGTGT GAAAGCAGCA |
| 361 | AAGAGGCACT GGCAGAAAAC AACCTGAACC TTCCAAAGAT GGCTGAAAAA GATGGATGCT |
| 421 | TCCAATCTGG ATTCAATGAG GAGACTTGCC TGGTGAAAAT CATCACTGGT CTTTTGGAGT |
| 481 | TTGAGGTATA CCTAGAGTAC CTCCAGAACA GATTTGAGAG TAGTGAGGAA CAAGCCAGAG |
| 541 | CTGTGCAGAT GAGTACAAAA GTCCTGATCC AGTTCCTGCA GAAAAAGGCA AAGAATCTAG |
| 601 | ATGCAATAAC CACCCCTGAC CCAACCACAA ATGCCAGCCT GCTGACGAAG CTGCAGGCAC |
| 661 | AGAACCAGTG GCTGCAGGAC ATGACAACTC ATCTCATTCT GCGCAGCTTT AAGGAGTTCC |
| 721 | TGCAGTCCAG CCTGAGGGCT CTTCGGCAAA TGTAGCATGG GCACCTCAGA TTGTTGTTGT |
| 781 | TAATGGGCAT TCCTTCTTCT GGTCAGAAAC CTGTCCACTG GGCACAGAAC TTATGTTGTT |
| 841 | CTCTATGGAG AACTAAAAGT ATGAGCGTTA GGACACTATT TTAATTATTT TTAATTTATT |
| 901 | AATATTTAAA TATGTGAAGC TGAGTTAATT TATGTAAGTC ATATTTATAT TTTTAAGAAG |
| 961 | TACCACTTGA AACATTTTAT GTATTAGTTT TGAAATAATA ATGGAAAGTG GCTATGCAGT |
| 1021 | TTGAATATCC TTTGTTTCAG AGCCAGATCA TTTCTTGGAA AGTGTAGGCT TACCTCAAAT |
| 1081 | AAATGGCTAA CTTATACATA TTTTTAAAGA AATATTTATA TTGTATTTAT ATAATGTATA |
| 1141 | AATGGTTTTT ATACCAATAA ATGGCATTTT AAAAAATTCA GCAAAAAAAA AAAAAAAAAA |
| 1201 | A |

By “interleukin 6 receptor (IL-6R) complex” is meant a protein complex comprising an IL-6 receptor subunit alpha (IL-6Rα) and interleukin 6 signal transducer Glycoprotein 130, also termed interleukin 6 receptor subunit β (IL-6Rβ).

By “interleukin 6 receptor subunit α (IL-6Rα) polypeptide” is meant a polypeptide or fragment thereof having at least about 85% or greater amino acid identity to the amino acid sequence provided at NCBI Accession No. NP_000556 or NP_852004 and having IL-6 receptor biological activity. Exemplary IL-6Rα biological activities include binding to IL-6, binding to glycoprotein 130 (gp130), and regulation of cell growth and differentiation. An exemplary IL-6R sequence is provided below:

| (SEQ ID NO: 7) | |
|---|---|
| 1 | MLAVGCALLA ALLAAPGAAL APRRCPAQEV ARGVLTSLPG DSVTLTCPGV EPEDNATVHW |
| 61 | VLRKPAAGSH PSRWAGMGRR LLLRSVQLHD SGNYSCYRAG RPAGTVHLLV DVPPEEPQLS |
| 121 | CFRKSPLSNV VCEWGPRSTP SLTTKAVLLV RKFQNSPAED FQEPCQYSQE SQKFSCQLAV |
| 181 | PEGDSSFYIV SMCVASSVGS KFSKTQTFQG CGILQPDPPA NITVTAVARN PRWLSVTWQD |
| 241 | PHSWNSSFYR LRFELRYRAE RSKTFTTWMV KDLQHHCVIH DAWSGLRHVV QLRAQEEFGQ |
| 301 | GEWSEWSPEA MGTPWTESRS PPAENEVSTP MQALTTNKDD DNILFRDSAN ATSLPVQDSS |
| 361 | SVPLPTFLVA GGSLAFGTLL CIAIVLRFKK TWKLRALKEG KTSMHPPYSL GQLVPERPRP |
| 421 | TPVLVPLISP PVSPSSLGSD NTSSHNRPDA RDPRSPYDIS NTDYFFPR |

By “interleukin 6 receptor subunit β (IL-6Rβ) polypeptide” is meant a polypeptide or fragment thereof having at least about 85% or greater amino acid identity to the amino acid sequence provided at NCBI Accession No. NP_002175, NP_786943, or NP_001177910 and having IL-6 receptor biological activity. Exemplary IL-6Rβ biological activities include binding to IL-6Rα, IL-6 receptor signaling activity, and regulation of cell growth, differentiation, hepcidin expression etc. An exemplary IL-6Rβ sequence is provided below:

| (SEQ ID NO: 8) | |
|---|---|
| 1 | MLTLQTWLVQ ALFIFLTTES TGELLDPCGY ISPESPVVQL HSNFTAVCVL KEKCMDYFHV |
| 61 | NANYIVWKTN HFTIPKEQYT IINRTASSVT FTDIASLNIQ LTCNILTFGQ LEQNVYGITI |
| 121 | ISGLPPEKPK NLSCIVNEGK KMRCEWDGGR ETHLETNFTL KSEWATHKFA DCKAKRDTPT |
| 181 | SCTVDYSTVY FVNIEVWVEA ENALGKVTSD HINFDPVYKV KPNPPHNLSV INSEELSSIL |
| 241 | KLTWTNPSIK SVIILKYNIQ YRTKDASTWS QIPPEDTAST RSSFTVQDLK PFTEYVFRIR |
| 301 | CMKEDGKGYW SDWSEEASGI TYEDRPSKAP SFWYKIDPSH TQGYRTVQLV WKTLPPFEAN |
| 361 | GKILDYEVTL TRWKSHLQNY TVNATKLTVN LTNDRYLATL TVRNLVGKSD AAVLTIPACD |
| 421 | FQATHPVMDL KAFPKDNMLW VEWTTPRESV KKYILEWCVL SDKAPCITDW QQEDGTVHRT |
| 481 | YLRGNLAESK CYLITVTPVY ADGPGSPESI KAYLKQAPPS KGPTVRTKKV GKNEAVLEWD |
| 541 | QLPVDVQNGF IRNYTIFYRT IIGNETAVNV DSSHTEYTLS SLTSDTLYMV RMAAYTDEGG |
| 601 | KDGPEFTFTT PKFAQGEIEA IVVPVCLAFL LTTLLGVLFC FNKRDLIKKH IWPNVPDPSK |
| 661 | SHIAQWSPHT PPRHNFNSKD QMYSDGNFTD VSVVEIEAND KKPFPEDLKS LDLFKKEKIN |
| 721 | TEGHSSGIGG SSCMSSSRPS ISSSDENESS QNTSSTVQYS TVVHSGYRHQ VPSVQVFSRS |
| 781 | ESTQPLLDSE ERPEDLQLVD HVDGGDGILP RQQYFKQNCS QHESSPDISH FERSKQVSSV |
| 841 | NEEDFVRLKQ QISDHISQSC GSGQMKMFQE VSAADAFGPG TEGQVERFET VGMEAATDEG |
| 901 | MPKSYLPQTV RQGGYMPQ |

By “IL-6 antagonist” is meant an agent that is capable of decreasing the biological activity of IL-6. IL-6 antagonists include agents that decrease the level of IL-6 polypeptide in serum, including agents that decrease the expression of an IL-6 polypeptide or nucleic acid; agents that decrease the ability of IL-6 to bind to the IL-6R; agents that decrease the expression of the IL-6R; and agents that decrease signal transduction by the IL-6R receptor when bound by IL-6. In preferred embodiments, the IL-6 antagonist decreases IL-6 biological activity by at least about 10%, 20%, 30%, 50%, 70%, 80%, 90%, 95%, or even 100%. As further described in Section 6.3.4 below, IL-6 antagonists include IL-6 binding polypeptides, such as anti-IL-6 antibodies and antigen binding fragments or derivatives thereof; IL-6R binding polypeptides, such as anti-IL-6R antibodies and antigen binding fragments or derivatives thereof; and synthetic chemical molecules, such as JAK1 and JAK3 inhibitors.

By “IL-6 antibody” or “anti-IL-6 antibody” is meant an antibody that specifically binds IL-6. Anti-IL-6 antibodies include monoclonal and polyclonal antibodies that are specific for IL-6, and antigen-binding fragments or derivatives thereof. IL-6 antibodies are described in greater detail in Section 8.3.6.1 below.

As used herein, “diuretic efficiency” is calculated as mmol urinary sodium per doubling of loop diuretic dose (mmol Na/doubling of loop diuretic dose) according to the methods described in Hanberg et al., Circ. Heart Fail. 2016; 9:e003180, the disclosure of which is incorporated herein by reference in its entirety.

By “diuretic resistant heart failure” is meant heart failure in which the patient's diuretic efficiency is less than 100.

The terms “biomarker” or “marker,” as used herein, refers to a molecule that can be detected. Therefore, a biomarker according to the present invention includes, but is not limited to, a nucleic acid, a polypeptide, a carbohydrate, a lipid, an inorganic molecule, an organic molecule, each of which may vary widely in size and properties. A “biomarker” can be a bodily substance relating to a bodily condition or disease. A “biomarker” can be detected using any means known in the art or by a previously unknown means that only becomes apparent upon consideration of the marker by the skilled artisan.

As used herein, “biomarker” in the context of the present invention encompasses, without limitation, proteins, nucleic acids, and metabolites, together with their polymorphisms, mutations, variants, modifications, subunits, fragments, protein-ligand complexes, and degradation products, elements, related metabolites, and other analytes or sample-derived measures. Biomarkers can also include mutated proteins or mutated nucleic acids. Biomarkers also encompass non-blood borne factors or non-analyte physiological markers of health status, such as clinical parameters, as well as traditional laboratory risk factors. As defined by the Food and Drug Administration (FDA), a biomarker is a characteristic (e.g. measurable DNA and/or RNA or a protein) that is “objectively measured and evaluated as an indicator of normal biologic processes, pathogenic processes, or pharmacologic responses to a therapeutic intervention or other interventions”. Biomarkers also include any calculated indices created mathematically or combinations of any one or more of the foregoing measurements, including temporal trends and differences. Biomarkers may be measured at any level spatial or temporal localization, including but not limited to within a tumor, within in a cell, or on the membrane of a cell.

By “agent” is meant any compound or composition suitable to be administered in therapy, and explicitly includes chemical compounds; proteins, including antibodies or antigen-binding fragments thereof; peptides; and nucleic acid molecules.

By “subject” or “individual” is meant a human or non-human mammal, including, but not limited to, bovine, equine, canine, ovine, feline, and rodent, including murine and rattus, subjects. A “patient” is a human subject.

As used herein, the terms “treat,” treating,” “treatment,” and the like refer to reducing or ameliorating a disorder, and/or signs or symptoms associated therewith, or slowing or halting the progression thereof. It will be appreciated that, although not precluded, treating a disorder or condition does not require that the disorder, condition or symptoms associated therewith be completely eliminated.

“Pre-treatment” means prior to the first administration of an IL-6 antagonist according the methods described herein. Pre-treatment does not exclude, and often includes, the prior administration of treatments other than an IL-6 antagonist, such as treatment with a diuretic, such as a loop diuretic.

By “biological sample” is meant any tissue, cell, fluid, or other material derived from an organism (e.g., human subject). In certain embodiments, the biological sample is serum, plasma, urine, or whole blood.

As used herein, an “instructional material” includes a publication, a recording, a diagram, or any other medium of expression which can be used to communicate the usefulness of a component of the invention in a kit for detecting biomarkers disclosed herein. The instructional material of the kit of the invention can, for example, be affixed to a container which contains the component of the invention or be shipped together with a container which contains the component. Alternatively, the instructional material can be shipped separately from the container with the intention that the instructional material and the component be used cooperatively by the recipient.

The “level” of one or more biomarkers means the absolute or relative amount or concentration of the biomarker in the sample as determined by measuring mRNA, cDNA, small organic molecules, nucleotides, ions or protein, or any portion thereof such as oligonucleotide or peptide. A level of a biomarker may refer, based on context, to a global level or a level within some subdivision of an organism or within a specific sample, by way of non-limiting example a level may refer to the amount or concentration of a biomarker in a urine sample or the level may refer to the amount or concentration of the same biomarker in a plasma sample.

“Measuring” or “measurement,” or alternatively “detecting” or “detection,” means determining the presence, absence, quantity or amount (which can be an effective amount) of either a given substance within a clinical or subject-derived sample, including the derivation of qualitative or quantitative concentration levels of such substances, or otherwise determining the values or categorization of a subject's clinical parameters.

A “reference level” of a biomarker means a level of a biomarker that is indicative of the presence or absence of a particular phenotype or characteristic. When the level of a biomarker in a subject is above the reference level of the biomarker it is indicative of the presence of, or relatively heightened level of, a particular phenotype or characteristic. When the level of a biomarker in a subject is below the reference level of the biomarker it is indicative of a lack of or relative lack of a particular phenotype or characteristic.

### Other Interpretational Conventions

Unless otherwise specified, antibody constant region residue numbering is according to the EU index as in Kabat.

Ranges: throughout this disclosure, various aspects of the invention can be presented in a range format. Ranges include the recited endpoints. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Unless specifically stated or apparent from context, as used herein the term “or” is understood to be inclusive.

Unless specifically stated or apparent from context, as used herein, the terms “a”, “an”, and “the” are understood to be singular or plural. That is, the articles “a” and “an” are used herein to refer to one or to more than one (i.e., to at least one) of the grammatical object of the article. By way of example, “an element” means one element or more than one element.

In this disclosure, “comprises,” “comprising,” “containing,” “having,” “includes,” “including,” and linguistic variants thereof have the meaning ascribed to them in U.S. Patent law, permitting the presence of additional components beyond those explicitly recited.

Unless specifically stated or otherwise apparent from context, as used herein the term “about” is understood as within a range of normal tolerance in the art, for example within 2 standard deviations of the mean and is meant to encompass variations of ±20% or ±10%, more preferably ±5%, even more preferably ±1%, and still more preferably ±0.1% from the stated value.

Where an antibody equilibrium dissociation constant (K_{D}) is reported, K_{D} is determined by surface plasmon resonance with the antibody (or antigen-binding fragment thereof) fixed to the chip surface with ligand flowed thereover.

### Summary of Experimental Observations

As further described below in Example 1, consecutive heart failure (“HF”) patients receiving high dose diuretic therapy at an outpatient treatment center were enrolled in a prospective observational study. Plasma levels of IL-6 were measured to query systemic associations of this pro-inflammatory cytokine with various disease parameters, and urine levels of IL-6 were measured to query IL-6 associations with local inflammation and neurohormonal activation at the level of renal tissues.

Plasma and urine IL-6 levels were only modestly correlated with one another.

Increases in urine levels of IL-6 were significantly correlated in these heart failure patients with measures of renal impairment, such as diuretic resistance, lower estimated glomerular filtration rate (“eGFR”), and increased tissue-level renin-angiotensin-aldosterone system (“RAAS”) activation.

Although an inverse association between diuretic efficiency and plasma IL-6 was also observed, upon adjustment for eGFR only urine IL-6 remained significantly associated with risk of low diuretic efficiency in these patients. Furthermore, when urine IL-6 and plasma IL-6 were both entered into a logistic regression model, only urine IL-6 remained associated with risk of low diuretic efficiency while plasma IL-6 showed no such association.

These data demonstrate that urine IL-6 level is a useful biomarker for renal inflammation, and can be used to gauge renal dysfunction in the setting of heart failure (cardiorenal syndrome). The data further suggest that serial measurements of urine IL-6 can be used to measure the renal benefits of treatments administered to patients with heart failure, notably heart failure patients with cardiorenal syndrome.

The urine IL-6 data, and to some extent the plasma IL-6 data, predict that treatment with an IL-6 antagonist should be effective to reduce renal inflammation in heart failure patients, that is, to treat symptoms of cardiorenal syndrome. However, because infection is often a precipitating cause of acute decompensation in heart failure patients, it is important to limit anti-cytokine and other immunosuppressive therapies to those heart failure patients who are likely to respond with improved renal and/or cardiac function. The cost of chronic IL-6 antagonist therapy also militates for limiting treatment to those heart failure patients who are likely to respond with improved renal and/or cardiac function.

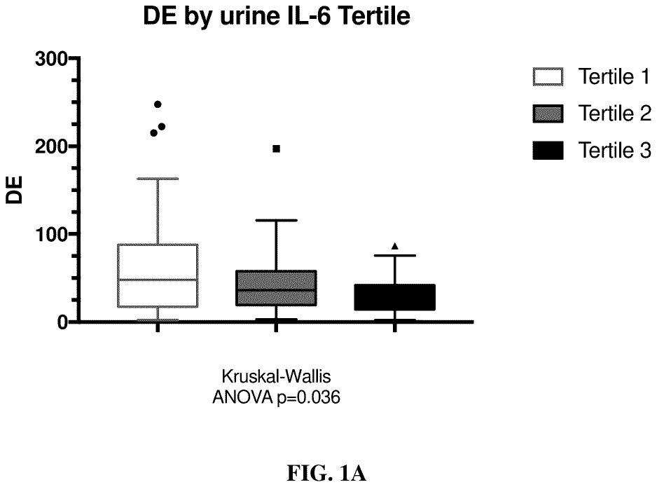

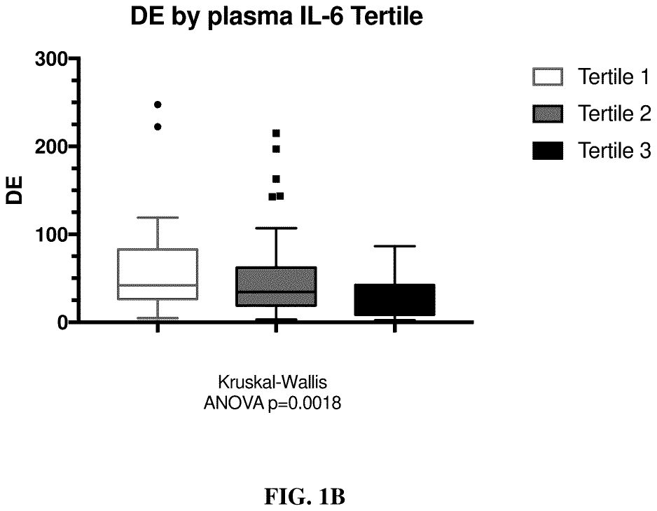

As detailed below in Example 2, the analysis conducted in Example 1 was expanded to 129 patients. FIG. 1A is a bar graph showing diuretic efficiency (“DE”) by tertiles of urine IL-6, confirming the inverse correlation of urinary IL-6 level with diuretic efficiency observed in the 98 patient subset. FIG. 1B is a bar graph showing diuretic efficiency (“DE”) by tertiles of plasma IL-6 in these 129 patients, confirming an inverse correlation of plasma IL-6 levels with diuretic efficiency.

The genotype of each of the 129 patients at the rs855791 single nucleotide polymorphism (“SNP”) in transmembrane protease serine 6 (“TMPRSS6”) was assessed. The TMPRSS6 polypeptide, also known as Matriptase-2 (MT2), cleaves hemojuvelin and inhibits bone morphogenetic protein signaling. The rs855791 (G2321A) SNP alters the TMPRSS6 protein sequence: the allele with highest frequency in the human population (the major allele) is 2321G, encoding 736A; the allele with lowest frequency in the human population (minor allele) is 2321A, encoding 736V.

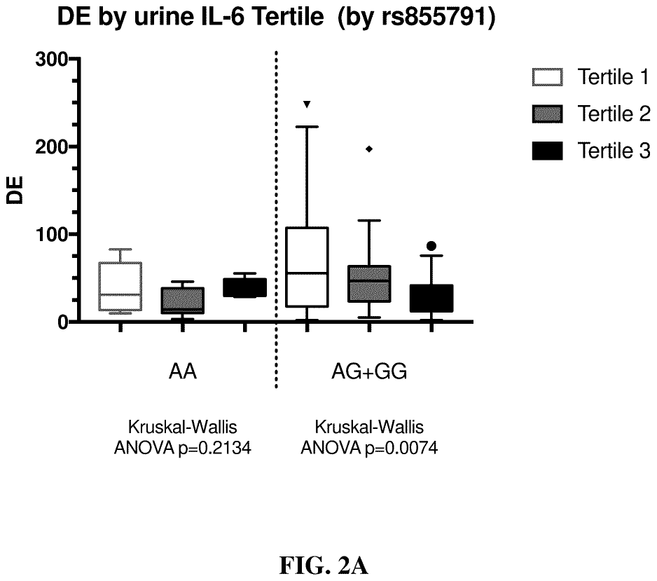

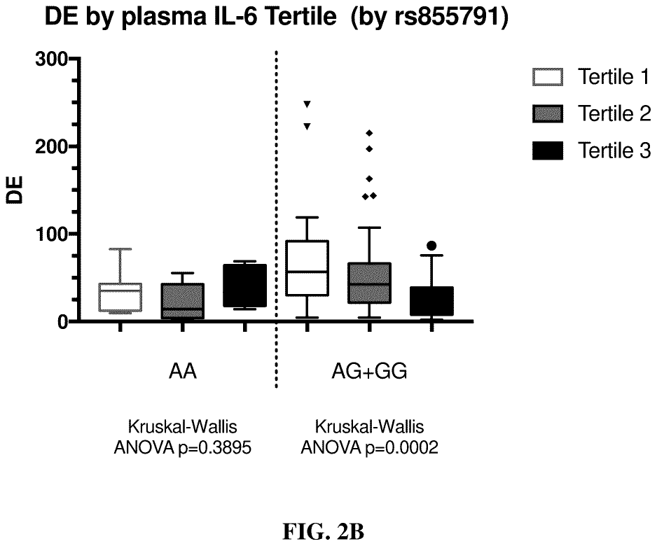

As shown in FIG. 2A, urine levels of IL-6 were inversely correlated with diuretic efficiency only in the patients having at least one copy of the major allele of the TMPRSS6 rs855791 SNP (FIG. 2A, right panel, “AG+GG”); urine levels of IL-6 are not significantly correlated with diuretic efficiency in patients homozygous for the minor allele (FIG. 2A, left panel, “AA”).

As shown in FIG. 2B, plasma levels of IL-6 correlated inversely with diuretic efficiency only in the patients having at least one copy of the major allele of the TMPRSS6 rs855791 SNP (FIG. 2B, right panel, “AG+GG”); plasma levels of IL-6 are not significantly correlated with diuretic efficiency in patients homozygous for the minor allele (FIG. 2B, left panel, “AA”).

These data suggested that diuretic resistance (low diuretic efficiency) in heart failure patients could be treated with an IL-6 antagonist, but only in those having at least one copy of the TMPRSS6 rs855791 major allele.

In mouse M1 CCD cells, which are genotypically analogous to human cells homozygous for the TMPRSS6 rs855791 major allele, the addition of IL-6 correlated with the expression of ion transporters, NKCC2, ENaC-beta, and NCC. Increased expression of these ion transporters provides a putative mechanism for IL-6 mediated diuretic resistance. And because the increased expression is not known to be linked to hepcidin expression, these data suggested that IL-6 antagonism could be effective in treating diuretic resistance even in patients homozygous for the TMPRSS6 rs855791 minor allele.

Secondary analysis of data from two large clinical trials in different heart failure patient populations confirmed the association of diuretic resistance with IL-6 level (Example 5), independently of TMPRSS6 rs855791 genotype (Example 6), providing evidence that IL-6 antagonism should also be effective in treating diuretic resistance in patients homozygous for the TMPRSS6 rs855791 minor allele.

### Methods of Treating Diuretic Resistance

Accordingly, in a first aspect, methods are provided for treating a patient who requires diuresis but is resistant to diuretics. The methods comprise administering, in combination with a diuretic, a therapeutically effective amount of an IL-6 antagonist to the patient.

In certain embodiments, the patient has elevated pre-treatment urine levels of IL-6. In some embodiments, the patient has elevated pre-treatment plasma IL-6 levels. In certain embodiments, the patient has elevated pre-treatment levels of IL-6 in urine and in plasma.

In some embodiments, the patient has diuretic-resistant heart failure. In various embodiments, the patient has cardiorenal syndrome.

In some embodiments, the patient has been determined to have at least one copy of the TMPRSS6 rs855791 major allele. In other embodiments, the patient is homozygous for the TMPRSS6 rs855791 minor allele.

### Diuretic Efficiency

In the methods described herein, the patient in need of the IL-6 antagonist treatment has a disease or condition that requires diuresis, and is diuretic resistant.

In certain embodiments, the patient has been treated or is being treated with a thiazide diuretic, such as chlorothiazide (Diuril®), chlorthalidone, hydrochlorothiazide (Microzide®), indapamide, or metolazone. In certain embodiments, the patient has been treated or is being treated with a loop diuretic, such as bumetanide (Bumex®), ethacrynic acid (Edecrin®), furosemide (Lasix®), or torsemide (Demadex®). In certain embodiments, the patient has been treated or is being treated with a potassium-sparing diuretic, such as amiloride, eplerenone (Inspra®), spironolactone (Aldactone®), or triamterene (Dyrenium®). In some embodiments, the patient has been treated or is being treated with more than one diuretic. In some embodiments, the patient has been treated or is being treated with a plurality of different types of diuretics.

By definition, the patient resistant to diuretics has a low diuretic efficiency. Diuretic efficiency is calculated as the increase in sodium output per doubling of the loop diuretic dose, centered on a dose of 40 mg of IV furosemide equivalents: diuretic efficiency=(mmol Na output)/(log₂(administered loop diuretic dose)−4.32). See Hanberg et al., Circ. Heart Fail. 2016; 9:e003180, the disclosure of which is incorporated herein by reference in its entirety.

In some embodiments, the patient has a diuretic efficiency of less than 500, such as less than 450, 400, 350, 300, 250, or 200. In some embodiment, the patient has a diuretic efficiency of less than 200, such as less than 195, 190, 185, 180, 175, 170, 165, 160, 155, or 150. In some embodiments, the patient has a diuretic efficiency of less than 150, such as less than 145, 140, 135, 130, 125, 120, 110, 105, or 100. In some embodiments, the patient has a diuretic efficiency of less than 100, such as less than 95, 90, 85, 80, 75, 70, 65, 60, 55, or 50. In particular embodiments, the patient has a diuretic efficiency of less than 50, such as less than 45, 40, 35, 30, or even less than 25, 20, 15, or 10.

In some embodiments, the patient resistant to diuretics requires a diuretic treatment of no less than 40 mg of furosemide (or equivalent) daily. In some of these embodiments, the patient requires a diuretic treatment of no less than 80 mg of furosemide (or equivalent) daily. In some of these embodiments, the patient requires a diuretic treatment of no less than 120 mg of furosemide (or equivalent) daily.

### Pre-Treatment IL-6 Levels

### Pre-Treatment Levels of IL-6 in Urine

In certain embodiments, the patient has pre-treatment urine levels of IL-6 of more than 5.0 pg IL-6/g creatinine, 6.0 pg IL-6/g creatinine, 7.0 pg IL-6/g creatinine, 8.0 pg IL-6/g creatinine, 9.0 pg IL-6/g creatinine, or 10.0 pg IL-6/g creatinine. In certain embodiments, the patient has pre-treatment IL-6 urine levels of more than 11.0 pg IL-6/g creatinine, 12.0 pg IL-6/g creatinine, 13.0 pg IL-6/g creatinine, 14.0 pg IL-6/g creatinine, or 15.0 pg IL-6/g creatinine. In further embodiments, the patient has pre-treatment IL-6 levels in urine of more than 16.0 pg IL-6/g creatinine, 17.0 pg IL-6/g creatinine, 18.0 pg IL-6/g creatinine, 19.0 pg IL-6/g creatinine, or 20.0 pg IL-6/g creatinine. In particular embodiments, the patient has pre-treatment IL-6 levels in urine of more than 21.0 pg IL-6/g creatinine, 22.0 pg IL-6/g creatinine, 23.0 pg IL-6/g creatinine, 24.0 pg IL-6/g creatinine, 25.0 pg IL-6/g creatinine, 26.0 pg IL-6/g creatinine, 27.0 pg IL-6/g creatinine, 28.0 pg IL-6/g creatinine, 29.0 pg IL-6/g creatinine, or even more than 30.0 pg IL-6/g creatinine. In certain embodiments, the patient has pre-treatment urine IL-6 levels of more than 35.0 pg/g creatinine.

In certain embodiments, the patient has pre-treatment urine levels of more than 14.2 pg IL-6/g creatinine (“elevated urine IL-6 levels”). In other embodiments, the patient has pre-treatment urine levels of less than 14.2 pg IL-/g creatinine.

In certain embodiments, the patient has levels of IL-6 in urine prior to treatment with an IL-6 antagonist and prior to treatment with a loop diuretic of more than 5.0 pg IL-6/g creatinine, 6.0 pg IL-6/g creatinine, 7.0 pg IL-6/g creatinine, 8.0 pg IL-6/g creatinine, 9.0 pg IL-6/g creatinine, or 10.0 pg IL-6/g creatinine. In some of these embodiments, the patient has IL-6 urine levels of more than 11.0 pg IL-6/g creatinine, 12.0 pg IL-6/g creatinine, 13.0 pg IL-6/g creatinine, 14.0 pg IL-6/g creatinine, or 15.0 pg IL-6/g creatinine. In further embodiments, the patient has levels of IL-6 in urine prior to treatment with an IL-antagonist and prior to treatment with a loop diuretic of more than 16.0 pg IL-6/g creatinine, 17.0 pg IL-6/g creatinine, 18.0 pg IL-6/g creatinine, 19.0 pg IL-6/g creatinine, or 20.0 pg IL-6/g creatinine. In particular embodiments, the patient has IL-6 levels in urine of more than 21.0 pg IL-6/g creatinine, 22.0 pg IL-6/g creatinine, 23.0 pg IL-6/g creatinine, 24.0 pg IL-6/g creatinine, 25.0 pg IL-6/g creatinine, 26.0 pg IL-6/g creatinine, 27.0 pg IL-6/g creatinine, 28.0 pg IL-6/g creatinine, 29.0 pg IL-6/g creatinine, or even more than 30.0 pg IL-6/g creatinine. In certain embodiments, the patient has urine IL-6 levels prior to treatment with an IL-antagonist and prior to treatment with a loop diuretic of more than 35.0 pg/g creatinine.

In certain embodiments, the patient has levels of IL-6 in urine prior to treatment with an IL-6 antagonist and prior to treatment with a loop diuretic of more than 14.2 pg IL-6/g creatinine. In other embodiments, the patient has levels of IL-6 in urine prior to treatment with an IL-antagonist and prior to treatment with a loop diuretic of less than 14.2 pg IL-/g creatinine.

### Pre-Treatment Levels of IL-6 in Plasma

In various embodiments, the patient has elevated pre-treatment plasma IL-6 levels.

In certain embodiments, the patient has pre-treatment plasma levels of IL-6 of more than 2.0 pg/mL. In other embodiments, the patient has pre-treatment plasma levels of IL-6 of less than 2.0 pg/mL.

In certain embodiments, the patient has pre-treatment plasma levels of IL-6 of more than 1.0 pg/ml, 1.1 pg/ml, 1.2 pg/ml, 1.3 pg/ml, 1.4 pg/ml, 1.5 pg/ml, 1.6 pg/ml, 1.7 pg/ml, 1.8 pg/ml, 1.9 pg/ml, or 2.0 pg/ml. In certain embodiments, the patient has pre-treatment plasma levels of IL-6 of more than 2.1 pg/ml, 2.2 pg/ml, 2.3 pg/ml, 2.4 pg/ml, 2.5 pg/ml, 2.6 pg/ml, 2.7 pg/ml, 2.8 pg/ml, 2.9 pg/ml, or 3.0 pg/ml. In certain embodiments, the patient has pre-treatment plasma levels of IL-6 of more than 3.1 pg/ml, 3.2 pg/ml, 3.3 pg/ml, 3.4 pg/ml, 3.5 pg/ml, 3.6 pg/ml, 3.7 pg/ml, 3.8 pg/ml, or 3.9 pg/ml.

In some embodiments, the patient has a pre-treatment IL-6 level of greater than 2 pg/mL, such as great than 3 pg/mL, 4 pg/mL, 5 pg/mL, 6 pg/mL, 8 pg/mL, 10 pg/mL, 15 pg/mL, or 20 pg/mL. In certain embodiments, the patient has a pre-treatment IL-6 level of greater than 3 pg/mL. In certain embodiments, the patient has a pre-treatment IL-6 level of greater than 5 pg/mL. In certain embodiments, the patient has a pre-treatment IL-6 level of greater than 10 pg/mL.

### Measurement of Pre-Treatment IL-6 Levels

Concentrations of IL-6 in urine, plasma, and serum can be determined using any standard assay known in the art. When IL-6 is measured in urine, the level may be indexed or normalized to another biomarker, in certain embodiments urinary creatinine.

In particular embodiments, concentrations are measured using the MesoScale Discovery (MSD) platform (Meso Scale diagnostics, Gaithersburg, MD, USA).

### Heart Failure

In typical embodiments of the methods described herein, the patient has heart failure.

In certain embodiments, the patient has NYHA functional class I heart failure. In certain embodiments, the patient has NYHA functional class II heart failure. In certain embodiments, the patient has NYHA functional class III heart failure. In certain embodiments, the patient has NYHA functional class IV heart failure.

In certain embodiments, the patient has acute heart failure. In certain embodiments, the patient has chronic heart failure.

In certain embodiments, the patient has a type of heart failure selected from Table 1 below.

TABLE 1

| ICD-10-CM | Description |
|---|---|
| I50 | Heart failure |
| | Heart failure complicating abortion or ectopic or |
| | molar pregnancy, heart failure following surgery, heart |
| | failure due to hypertension, heart failure due to |
| | hypertension with chronic kidney disease, obstetic |
| | surgery and procedures, rheumatic heart failure |
| I50.9 | Heart failure, unspecified |
| | Biventricular (heart) failure NOS |
| | Cardiac, heart or myocardial failure NOS |
| | Congestive heart disease |
| | Congestive heart failure |
| | Right ventricular failure |
| | (secondary to left heart failure) |
| I50.1 | Left ventricular failure |
| | Cardiac asthma |
| | Edema of lung with heart disease NOS |
| | Edema of lung with heart failure |
| | Left heart failure |
| | Pulmonary edema with heart disease NOS |
| | Pulmonary edema with heart failure |
| I50.20 | Unspecified systolic (congestive) heart failure |
| I50.21 | Acute systolic (congestive) heart failure |
| I50.22 | Chronic systolic (congestive) heart failure |
| I50.23 | Acute on chronic systolic (congestive) heart failure |
| I50.30 | Unspecified diastolic (congestive) heart failure |
| I50.31 | Acute diastolic (congestive) heart failure |
| I50.32 | Chronic diastolic (congestive) heart failure |
| I50.33 | Acute on chronic diastolic (congestive) heart failure |
| I50.40 | Unspecified combined systolic (congestive) and |
| | diastolic (congestive) heart failure |
| I50.41 | Acute combined systolic (congestive) and |
| | diastolic (congestive) heart failure |
| I50.42 | Chronic combined systolic (congestive) and |
| | diastolic (congestive) heart failure |
| I50.43 | Acute on chronic combined systolic (congestive) and |
| | diastolic (congestive) heart failure |
| I50.1 | Left ventricular failure |
| | Heart failure, unspecified |
| | Biventricular (heart) failure NOS |
| | Cardiac, heart or myocardial failure NOS |
| | Congestive heart disease |
| | Congestive heart failure |
| | Right ventricular failure |
| | (secondary to left heart failure) |

In certain embodiments, the patient has cardiorenal syndrome. In particular embodiments, the patient has cardiorenal syndrome type 1. In particular embodiments, the patient has cardiorenal syndrome type 2. In particular embodiments, the patient has cardiorenal syndrome type 3. In particular embodiments, the patient has cardiorenal syndrome type 4.

In certain embodiments, the patient has diuretic resistant heart failure. In certain of these embodiments, the heart failure patient has a diuretic efficiency of less than 100, 95, 90, 85, 80, 75, 70, 65, 60, 55, or 50. In particular embodiments, the patient has a diuretic efficiency of less than 45, 40, 35, 30, or even less than 25, 20, 15, or 10.

### Kidney Disease

In some embodiments of methods described herein, the patient has kidney disease.

In certain embodiments, the patient has diuretic resistant kidney disease. In certain of these embodiments, the kidney disease patient has a diuretic efficiency of less than 100, 95, 90, 85, 80, 75, 70, 65, 60, 55, or 50. In particular embodiments, the patient has a diuretic efficiency of less than 45, 40, 35, 30, or even less than 25, 20, 15, or 10.

In particular embodiments, the patient has hepatorenal syndrome.

TMPRSS6 rs855791 Genotype

In certain embodiments, the patient has previously been determined to have at least one copy of the TMPRSS6 rs855791 major allele. In other embodiments, the method further comprises the earlier step of determining that the patient has at least one copy of the TMPRSS6 rs855791 major allele.

Preferably, the genotype at both alleles is determined, thus permitting identification and discrimination of patients who are homozygous for the TMPRSS6 rs855791 major allele, heterozygous for the major and minor TMPRSS6 rs855791 alleles, and homozygous for the TMPRSS6 rs855791 minor allele.

The absence (major allele) or presence (minor allele) of SNP rs855791 (2321G→A) in the TMPRSS6 gene is determined using standard techniques.

Typically, PCR is used to amplify a biological sample obtained from the patient.

In certain embodiments, the absence or presence of polymorphism is detected concurrently with amplification using real-time PCR (RT-PCR). In certain embodiments, the RT-PCR assay employs 5′ nuclease (TaqMan® probes), molecular beacons, and/or FRET hybridization probes. Reviewed in Espy et al., Clin. Microbiol. Rev. 2006 January; 19(1): 165-256, incorporated herein by reference in its entirety. In typical embodiments, a commercially available assay is used. In select embodiments, the commercially available assay is selected from the group consisting of TaqMan™ SNP Genotyping Assays (ThermoFisher); PCR SNP Genotyping Assay (Qiagen); Novallele Genotyping Assays (Canon); and SNP Type™ assays (formerly SNPtype) (Fluidigm).

In certain embodiments, the absence or presence of polymorphism is detected following amplification using hybridization with a probe specific for SNP rs855791, restriction endonuclease digestion, nucleic acid sequencing, primer extension, microarray or gene chip analysis, mass spectrometry, and/or a DNAse protection assay. In certain embodiments, the allelic variants are called by sequencing. In certain embodiments, Sanger sequencing is used. In certain embodiments, one of a variety of next-generation sequencing techniques is used, including for example a sequencing technique selected from the group consisting of microarray sequencing, Solexa sequencing (Illumina), Ion Torrent (Life Technologies), SOliD (Applied Biosystems), pyrosequencing, single-molecule real-time sequencing (Pacific Bio), nanopore sequencing and tunneling currents sequencing.

In certain embodiments, the absence or presence of polymorphisms is detected using the procedures set forth in Example 2 below.

### IL-6 Antagonists

The IL-6 antagonist used in the methods described herein is capable of decreasing the biological activity of IL-6.

### Anti-IL-6 Antibodies

In certain embodiments, the IL-6 antagonist is an anti-IL-6 antibody or antigen-binding fragment or derivative thereof.

In certain embodiments, the IL-6 antagonist is a full-length anti-IL-6 monoclonal antibody. In particular embodiments, the full-length monoclonal antibody is an IgG antibody. In certain embodiments, the full-length monoclonal antibody is an IgG1, IgG2, IgG3, or IgG4 antibody. In certain embodiments, the IL-6 antagonist is a polyclonal composition comprising a plurality of species of full-length anti-IL-6 antibodies, each of the plurality having unique CDRs. In certain embodiments, the IL-6 antagonist is an antibody fragment selected from Fab, Fab′, and F(ab′)2 fragments. In certain embodiments, the IL-6 antagonist is a scFv, a disulfide-linked Fv (dsFv), or a single domain antibody, such as a camelid-derived VHH single domain Nanobody. In certain embodiments, the IL-6 antagonist is immunoconjugate or fusion comprising an IL-6 antigen-binding fragment. In certain embodiments, the antibody is bispecific or multispecific, with at least one of the antigen-binding portions having specificity for IL-6.

In certain embodiments, the antibody is fully human. In certain embodiments, the antibody is humanized. In certain embodiments, the antibody is chimeric and has non-human V regions and human C region domains. In certain embodiments, the antibody is murine.

In typical embodiments, the anti-IL-6 antibody has a K_{D} for binding human IL-6 of less than 100 nM. In certain embodiments, the anti-IL-6 antibody has a K_{D} for binding human IL-6 of less than 75 nM, 50 nM, 25 nM, 20 nM, 15 nM, or 10 nM. In particular embodiments, the anti-IL-6 antibody has a K_{D} for binding human IL-6 of less than 5 nM, 4 nM, 3 nM, or 2 nM. In selected embodiments, the anti-IL-6 antibody has a K_{D} for binding human IL-6 of less than 1 nM, 750 pM, or 500 pM. In specific embodiments, the anti-IL-6 antibody has a K_{D} for binding human IL-6 of no more than 500 pM, 400 pM, 300 pM, 200 pM, or 100 pM.

In typical embodiments, the anti-IL-6 antibody neutralizes the biological activity of IL-6. In certain embodiments, the neutralizing antibody prevents binding of IL-6 to the IL-6 receptor.

In typical embodiments, the anti-IL-6 antibody has an elimination half-life following intravenous administration of at least 7 days. In certain embodiments, the anti-IL-6 antibody has an elimination half-life of at least 14 days, at least 21 days, or at least 30 days.

In certain embodiments, the anti-IL-6 antibody has a human IgG constant region with at least one amino acid substitution that extends serum half-life as compared to the unsubstituted human IgG constant domain.

In certain embodiments, the IgG constant domain comprises substitutions at residues 252, 254, and 256, wherein the amino acid substitution at amino acid residue 252 is a substitution with tyrosine, the amino acid substitution at amino acid residue 254 is a substitution with threonine, and the amino acid substitution at amino acid residue 256 is a substitution with glutamic acid (“YTE”). See U.S. Pat. No. 7,083,784, incorporated herein by reference in its entirety. In certain extended half-life embodiments, the IgG constant domain comprises substitutions selected from T250Q/M428L (Hinton et al., J. Immunology 176:346-356 (2006)); N434A (Yeung et al., J. Immunology 182:7663-7671 (2009)); or T307A/E380A/N434A (Petkova et al., International Immunology, 18: 1759-1769 (2006)).

In certain embodiments, the elimination half-life of the anti-IL-6 antibody is increased by utilizing the FcRN-binding properties of human serum albumin. In certain embodiments, the antibody is conjugated to albumin (Smith et al., Bioconjug. Chem., 12: 750-756 (2001)). In certain embodiments, the anti-IL-6 antibody is fused to bacterial albumin-binding domains (Stork et al., Prot. Eng. Design Science 20: 569-76 (2007)). In certain embodiments, the anti-IL-6 antibody is fused to an albumin-binding peptide (Nguygen et al., Prot Eng Design Sel 19: 291-297 (2006)). In certain embodiments, the anti-IL-antibody is bispecific, with one specificity being to IL-6, and one specificity being to human serum albumin (Ablynx, WO 2006/122825 (bispecific Nanobody)).

In certain embodiments, the elimination half-life of the anti-IL-6 antibody is increased by PEGylation (Melmed et al., Nature Reviews Drug Discovery 7: 641-642 (2008)); by HPMA copolymer conjugation (Lu et al., Nature Biotechnology 17: 1101-1104 (1999)); by dextran conjugation (Nuclear Medicine Communications, 16: 362-369 (1995)); by conjugation with homo-amino-acid polymers (HAPs; HAPylation) (Schlapschy et al., Prot Eng Design Sel 20: 273-284 (2007)); or by polysialylation (Constantinou et al., Bioconjug. Chem. 20: 924-931 (2009)).

### MEDI5117 and Derivatives

In certain embodiments, the anti-IL-6 antibody or antigen-binding portion thereof comprises all six CDRs of MEDI5117. In particular embodiments, the antibody or antigen-binding portion thereof comprises the MEDI5117 heavy chain V region and light chain V region. In specific embodiments, the antibody is the full-length MEDI5117 antibody. The MEDI5117 antibody is described in WO 2010/088444 and US 2012/0034212, the disclosures of which are incorporated herein by reference in their entireties. The MEDI5117 antibody has the following CDR and heavy and light chain sequences:

MEDI5117 VH CDR1
(SEQ ID NO: 9)
SNYMI
MEDI5117 VH CDR2
(SEQ ID NO: 10)
DLYYYAGDTYYADSVKG
MEDI5117 VH CDR3
(SEQ ID NO: 11)
WADDHPPWIDL
MEDI5117 VL CDR1
(SEQ ID NO: 12)
RASQGISSWLA
MEDI5117 VL CDR2
(SEQ ID NO: 13)
KASTLES
MEDI5117 VL CDR3
(SEQ ID NO: 14)
QQSWLGGS
MEDI5117 Heavy chain
(SEQ ID NO: 15)
EVQLVESGGGLVQPGGSLRLSCAASGFTISSNYMIWVRQAPGKGLEWVSD
LYYYAGDTYYADSVKGRFTMSRDISKNTVYLQMNSLRAEDTAVYYCARWA
DDHPPWIDLWGRGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLYITREPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
MEDI5117 Light chain
(SEQ ID NO: 16)
DIQMTQSPSTLSASVGDRVTITCRASQGISSWLAWYQQKPGKAPKVLIYK
ASTLESGVPSRFSGSGSGTEFTLTISSLQPDDFATYYCQQSWLGGSFGQG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

In certain embodiments, the anti-IL-6 antibody is a derivative of MED5117.

In certain embodiments, the MED5117 derivative includes one or more amino acid substitutions in the MED5117 heavy and/or light chain V regions.

In certain embodiments, the derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original VH and/or VL of the MEDI5117 anti-IL-6 antibody, while retaining specificity for human IL-6.

In certain embodiments, the MED5117 derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the VH and VL domain of MEDI5117. The percent sequence identity is determined using BLAST algorithms using default parameters.

In certain embodiments, the MED5117 derivative comprises an amino acid sequence in which the CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the respective CDRs of MEDI5117. The percent sequence identity is determined using BLAST algorithms using default parameters.

In certain embodiments, the VH and/or VL CDR derivatives comprise conservative amino acid substitutions at one or more predicted nonessential amino acid residues (i.e., amino acid residues which are not critical for the antibody to specifically bind to human IL-6).

### Other Anti-IL-6 Antibodies

In certain embodiments, the anti-IL-6 antibody comprises the six CDRs from an antibody selected from the group consisting of siltuximab, gerilimzumab, sirukumab, clazakizumab, olokizumab, elsilimomab, VX30 (VOP-R003; Vaccinex), EB-007 (EBI-029; Eleven Bio), ARGX-109 (ArGEN-X), FM101 (Femta Pharmaceuticals, Lonza) and ALD518/BMS-945429 (Alder Biopharmaceuticals, Bristol-Myers Squibb). In certain embodiments, the anti-IL-6 antibody comprises the heavy chain V region and light chain V region from an antibody selected from the group consisting of siltuximab, gerilimzumab, sirukumab, clazakizumab, olokizumab, VX30 (VOP-R003; Vaccinex), EB-007 (EBI-029; Eleven Bio), ARGX-109 (ArGEN-X), FM101 (Femta Pharmaceuticals, Lonza) and ALD518/BMS-945429 (Alder Biopharmaceuticals, Bristol-Myers Squibb). In particular embodiments, the anti-IL-6 antibody is an antibody selected from the group consisting of siltuximab, gerilimzumab, sirukumab, clazakizumab, olokizumab, VX30 (VOP-R003; Vaccinex), EB-007 (EBI-029; Eleven Bio), ARGX-109 (ArGEN-X), FM101 (Femta Pharmaceuticals, Lonza) and ALD518/BMS-945429 (Alder Biopharmaceuticals, Bristol-Myers Squibb).

In certain embodiments, the anti-IL-6 antibody comprises the six CDRs from an antibody selected from those described in US 2016/0168243, US 2016/0130340, US 2015/0337036, US 2015/0203574, US 2015/0140011, US 2015/0125468, US 2014/0302058, US 2014/0141013, US 2013/0280266, US 2013/0017575, US 2010/0215654, US 2008/0075726, U.S. Pat. No. 5,856,135, US 2006/0240012, US 2006/0257407, or U.S. Pat. No. 7,291,721, the disclosures of which are incorporated herein by reference in their entireties.

### Anti-IL-6 Receptor Antibodies

In certain embodiments, the IL-6 antagonist is an anti-IL-6 receptor antibody or antigen-binding fragment or derivative thereof.

In certain embodiments, the IL-6 antagonist is a full-length anti-IL-6 receptor monoclonal antibody. In particular embodiments, the full-length monoclonal antibody is an IgG antibody. In certain embodiments, the full-length monoclonal antibody is an IgG1, IgG2, IgG3, or IgG4 antibody. In certain embodiments, the IL-6 antagonist is a polyclonal composition comprising a plurality of species of full-length anti-IL-6 receptor antibodies, each of the plurality having unique CDRs. In certain embodiments, the IL-6 antagonist is an antibody fragment selected from Fab and Fab′ fragments. In certain embodiments, the IL-6 antagonist is a scFv, a single domain antibody, including a camelid-derived VHH single domain Nanobody. In certain embodiments, the antibody is bispecific or multispecific, with at least one of the antigen-binding portions having specificity for IL-6R.

In certain embodiments, the antibody is fully human. In certain embodiments, the antibody is humanized. In certain embodiments, the antibody is chimeric and has non-human V regions and human C region domains. In certain embodiments, the antibody is murine.

In typical embodiments, the anti-IL-6 receptor antibody has a K_{D} for binding human IL-6R of less than 100 nM. In certain embodiments, the anti-IL-6R antibody has a K_{D} for binding human IL-6R of less than 75 nM, 50 nM, 25 nM, 20 nM, 15 nM, or 10 nM. In particular embodiments, the anti-IL-6 receptor antibody has a K_{D} for binding human IL-6R of less than 5 nM, 4 nM, 3 nM, or 2 nM. In selected embodiments, the anti-IL-6 receptor antibody has a K_{D} for binding human IL-6R of less than 1 nM, 750 pM, or 500 pM. In specific embodiments, the anti-IL-6 receptor antibody has a K_{D} for binding human IL-6R of no more than 500 pM, 400 pM, 300 pM, 200 pM, or 100 pM.

In typical embodiments, the anti-IL-6R reduces the biological activity of IL-6.

In typical embodiments, the anti-IL-6R antibody has an elimination half-life following intravenous administration of at least 7 days. In certain embodiments, the anti-IL-6R antibody has an elimination half-life of at least 14 days, at least 21 days, or at least 30 days.

In certain embodiments, the anti-IL-6R antibody has a human IgG constant region with at least one amino acid substitution that extends serum half-life as compared to the unsubstituted human IgG constant domain.

In certain embodiments, the IgG constant domain comprises substitutions at residues 252, 254, and 256, wherein the amino acid substitution at amino acid residue 252 is a substitution with tyrosine, the amino acid substitution at amino acid residue 254 is a substitution with threonine, and the amino acid substitution at amino acid residue 256 is a substitution with glutamic acid (“YTE”). See U.S. Pat. No. 7,083,784, incorporated herein by reference in its entirety. In certain extended half-life embodiments, the IgG constant domain comprises substitutions selected from T250Q/M428L (Hinton et al., J. Immunology 176:346-356 (2006)); N434A (Yeung et al., J. Immunology 182:7663-7671 (2009)); or T307A/E380A/N434A (Petkova et al., International Immunology, 18: 1759-1769 (2006)).

In certain embodiments, the elimination half-life of the anti-IL-6R antibody is increased by utilizing the FcRN-binding properties of human serum albumin. In certain embodiments, the antibody is conjugated to albumin (Smith et al, Bioconjug. Chem., 12: 750-756 (2001)). In certain embodiments, the anti-IL-6R antibody is fused to bacterial albumin-binding domains (Stork et al., Prot. Eng. Design Science 20: 569-76 (2007)). In certain embodiments, the anti-IL-6 antibody is fused to an albumin-binding peptide (Nguygen et al., Prot Eng Design Sel 19: 291-297 (2006)). In certain embodiments, the anti-IL-antibody is bispecific, with one specificity being to IL-6R, and one specificity being to human serum albumin (Ablynx, WO 2006/122825 (bispecific Nanobody)).

In certain embodiments, the elimination half-life of the anti-IL-6R antibody is increased by PEGylation (Melmed et al., Nature Reviews Drug Discovery 7: 641-642 (2008)); by HPMA copolymer conjugation (Lu et al., Nature Biotechnology 17: 1101-1104 (1999)); by dextran conjugation (Nuclear Medicine Communications, 16: 362-369 (1995)); by conjugation with homo-amino-acid polymers (HAPs; HAPylation) (Schlapschy et al., Prot Eng Design Sel 20: 273-284 (2007)); or by polysialylation (Constantinou et al., Bioconjug. Chem. 20: 924-931 (2009)).

In certain embodiments, the anti-IL-6R antibody or antigen-binding portion thereof comprises all six CDRs of tocilizumab. In particular embodiments, the antibody or antigen-binding portion thereof comprises the tocilizumab heavy chain V region and light chain V region. In specific embodiments, the antibody is the full-length tocilizumab antibody.

In certain embodiments, the anti-IL-6R antibody or antigen-binding portion thereof comprises all six CDRs of sarilumab. In particular embodiments, the antibody or antigen-binding portion thereof comprises the sarilumab heavy chain V region and light chain V region. In specific embodiments, the antibody is the full-length sarilumab antibody.

In certain embodiments, the anti-IL-6R antibody or antigen-binding portion thereof comprises all six CDRs of VX30 (Vaccinex), ARGX-109 (arGEN-X), FM101 (Formatech), SA237 (Roche), NI-1201 (NovImmune), or an antibody described in US 2012/0225060.

In certain embodiments, the anti-IL-6R antibody or antigen-binding portion thereof is a single domain antibody. In particular embodiments, the single domain antibody is a camelid VHH single domain antibody. In specific embodiments, the antibody is vobarilizumab (ALX-0061) (Ablynx NV).

### Anti-IL-6:IL-6R Complex Antibodies

In certain embodiments, the IL-6 antagonist is an antibody specific for the complex of IL-6 and IL-6R. In certain embodiments, the antibody has the six CDRs of an antibody selected from those described in US 2011/0002936, which is incorporated herein by reference in its entirety.

### JAK and STAT Inhibitors

IL-6 is known to signal via the JAK-STAT pathway.

In certain embodiments, the IL-6 antagonist is an inhibitor of the JAK signaling pathway. In certain embodiments, the JAK inhibitor is a JAK1-specific inhibitor. In certain embodiments, the JAK inhibitor is a JAK3-specific inhibitor. In certain embodiments, the JAK inhibitor is a pan-JAK inhibitor.

In certain embodiments, the JAK inhibitor is selected from the group consisting of tofacitinib (Xeljanz), decernotinib, ruxolitinib, upadacitinib, baricitinib, filgotinib, lestaurtinib, pacritinib, peficitinib, INCB-039110, ABT-494, INCB-047986 and AC-410.

In certain embodiments, the IL-6 antagonist is a STAT3 inhibitor. In a specific embodiment, the inhibitor is AZD9150 (AstraZeneca, Isis Pharmaceuticals), a STAT3 antisense molecule.

### Additional IL-6 Antagonists

In certain embodiments, the IL-6 antagonist is an antagonist peptide.

In certain embodiments, the IL-6 antagonist is C326 (an IL-6 antagonist by Avidia, also known as AMG220), or FE301, a recombinant protein inhibitor of IL-6 (Ferring International Center S.A., Conaris Research Institute AG). In certain embodiments, the anti-IL-6 antagonist comprises soluble gp130, FE301 (Conaris/Ferring).

### Dosage Regimens

### Antibodies, Antigen-Binding Fragments, Peptides

In typical embodiments, antibody, antigen-binding fragments, and peptide IL-6 antagonists are administered parenterally.

In some parenteral embodiments, the IL-6 antagonist is administered intravenously. In certain intravenous embodiments, the IL-6 antagonist is administered as a bolus. In certain intravenous embodiments, the IL-6 antagonist is administered as an infusion. In certain intravenous embodiments, the IL-6 antagonist is administered as a bolus followed by infusion. In some parenteral embodiments, the IL-6 antagonist is administered subcutaneously.

In certain embodiments, the antibody, antigen-binding fragment, or peptide IL-6 antagonist is administered in a dose that is independent of patient weight or surface area (flat dose).

In certain embodiments, the intravenous flat dose is 1 mg, 2 mg, 3 mg, 4 mg, 5 mg, 6 mg, 7 mg, 8 mg, 9 mg, or 10 mg. In certain embodiments, the intravenous flat dose is 11 mg, 12 mg, 13 mg, 14 mg, 15 mg, 16 mg, 17 mg, 18 mg, 19 mg, or 20 mg. In certain embodiments, the intravenous flat dose is 25 mg, 30 mg, 40 mg, or 50 mg. In certain embodiments, the intravenous flat dose is 60 mg, 70 mg, 80 mg, 90 mg, or 100 mg. In certain embodiments, the intravenous flat dose is 1-10 mg, 10-15 mg, 15-20 mg, 20-30 mg, 30-40 mg, or 40-50 mg. In certain embodiments, the intravenous flat dose is 1-40 mg, or 50-100 mg.

In certain embodiments, the subcutaneous flat dose is 10 mg, 20 mg, 30 mg, 40 mg, 50 mg, 60 mg, 70 mg, 80 mg, 90 mg, or 100 mg. In certain embodiments, the subcutaneous flat dose is 110 mg, 120 mg, 130 mg, 140 mg, 150 mg, 160 mg, 170 mg, 180 mg, 190 mg, or 200 mg. In certain embodiments, the subcutaneous flat dose is 210 mg, 220 mg, 230 mg, 240 mg, or 250 mg. In certain embodiments, the subcutaneous flat dose is 10-100 mg, 100-200 mg, or 200-250 mg. In certain embodiments, the subcutaneous flat dose is 10-20 mg, 20-30 mg, 30-40 mg, 40-50 mg, 50-60 mg, 60-70 mg, 70-80 mg, 80-90 mg, or 90-100 mg. In certain embodiments, the subcutaneous flat dose is 100-125 mg, 125-150 mg, 150-175 mg, 175-200 mg, or 200-250 mg.

In certain embodiments, the antibody, antigen-binding fragment, or peptide IL-6 antagonist is administered as a patient weight-based dose.

In certain embodiments, the antagonist is administered at an intravenous dose of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg or 1.0 mg/kg. In certain embodiments, the antagonist is administered at a dose of 1.5 mg/kg, 2 mg/kg, 2.5 mg/kg, 3 mg/kg, 3.5 mg/kg, 4 mg/kg, 4.5 mg/kg, or 5 mg/kg.

In certain embodiments, the subcutaneous weight-based dose is 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg or 1.0 mg/kg. In certain embodiments, the antagonist is administered at a dose of 1.5 mg/kg, 2 mg/kg, 2.5 mg/kg, 3 mg/kg, 3.5 mg/kg, 4 mg/kg, 4.5 mg/kg, or 5 mg/kg.

In various intravenous embodiments, the IL-6 antagonist is administered once every 7 days, once every 14 days, once every 21 days, once every 28 days, or once a month. In various subcutaneous embodiments, the IL-6 antagonist is administered once every 14 days, once every 28 days, once a month, once every two months (every other month), or once every three months.

In certain preferred embodiments, the IL-6 antagonist is the MEDI5117 antibody. In certain embodiments, MEDI5117 is administered in a flat dose of 1-30 mg IV once every week. In certain embodiments, the MEDI5117 antibody is administered in a flat dose of 1, 2, 3, 4, 5, 7.5, 10, 15, 20, 25, or 30 mg IV once every week. In certain embodiments, the MEDI5117 antibody is administered in a flat dose of 25-250 mg s.c. once every month to once every three months. In particular embodiments, MEDI5117 is administered at a dose of 30 mg, 45 mg, 60 mg, 75 mg, 100 mg, 120 mg, 125 mg, 150 mg, 175 mg, 200 mg, 225 mg, 240 mg, or 250 mg s.c. once every month, once every two months, or once every 3 months.

In certain embodiments, the IL-6 antagonist is tocilizumab. In certain embodiments, tocilizumab is administered s.c. in a starting dose for patients ≥100 kg of 162 mg once every week. In certain embodiments, tocilizumab is administered intravenously at a dose of 4 mg/kg once every 4 weeks followed by an increase to 8 mg/kg every 4 weeks based on clinical response.

### JAK and STAT Inhibitors

In typical embodiments, small molecule JAK inhibitors and STAT inhibitors are administered orally.

In certain embodiments, the inhibitor is administered once or twice a day at an oral dose of 1-10 mg, 10-20 mg, 20-30 mg, 30-40 mg, or 40-50 mg. In certain embodiments, the inhibitor is administered once or twice a day at a dose of 50-60 mg, 60-70 mg, 70-80 mg, 80-90 mg, or 90-100 mg. In certain embodiments, the inhibitor is administered at a dose of 5, 10, 15, 20, 25, 30, 35, 40, 45, or 50 mg PO once or twice a day. In certain embodiments, the inhibitor is administered at a dose of 75 mg PO QD or BID, 100 mg PO QD or BID.

In certain embodiments, the JAK inhibitor is tofacitinib, and is administered at a dose of 5 mg PO BID or 11 mg PO GqDay,

In certain embodiments, the JAK inhibitor is decernotinib, and is administered at a dose of 25 mg, 50 mg, 100 mg, or 150 mg PO BID.

In certain embodiments, the inhibitor is ruxolitinib, and is administered at dose of 25 mg PO BID, 20 mg PO BID, 15 mg PO BID, 10 mg PO BID, or 5 mg PO BID.

### Treating to Goal

In certain embodiments, the IL-6 antagonist is administered at a dose, on a schedule, and for a period sufficient to increase diuretic efficiency. In certain of these embodiments, the IL-6 antagonist is administered at a dose, on a schedule, and for a period sufficient to increase diuretic efficiency to normal levels.

In certain embodiments, the IL-6 antagonist is administered at a dose, on a schedule, and for a period sufficient to increase eGFR, and in certain of these embodiments, the IL-6 antagonist is administered at a dose, on a schedule, and for a period sufficient to increase eGFR to normal levels.

### IL-6 Level Monitoring

In certain embodiments, the impact of treatment with IL-6 antagonists on cardiorenal parameters may be monitored by measuring a level of IL-6 in a urine or plasma sample from the patient. It is specifically contemplated that the methods of the invention may be used to monitor the efficacy of treatments for cardiorenal syndrome and may motivate, for example, a change of dose or therapeutic.

Accordingly, in certain embodiments, the method further comprises the subsequent step of determining the level of IL-6 in urine, in plasma, or in both urine and plasma. In certain of these embodiments, the method further comprises a final step of adjusting the dose of at least one subsequent administration of IL-6 antagonist based on IL-6 level determined in the immediately preceding step.

### Additional Therapeutic Agents

In certain embodiments of the methods described herein, the method further comprises administering at least one therapeutic agent additional to the IL-6 antagonist, wherein the additional therapeutic agent treats one or more cardiovascular or renal symptoms of heart failure. Specific treatment will be determined on a case by case basis by the attending physician.

### Standard Heart Failure Agents

In certain embodiments, the additional therapeutic agent is a diuretic.

In particular embodiments, the diuretic is a loop diuretic. In select embodiments, the loop diuretic is selected from the group consisting of furosemide, torsemide, bumetanide, and ethacrynic acid. In particular embodiments, the loop diuretic is furosemide. In certain embodiments, furosemide is administered orally. In certain embodiments, furosemide is administered intravenously. In certain embodiments, the diuretic is a thiazide diuretic. In particular embodiments, the thiazide diuretic is chlorothiazide, hydrochlorothiazide, chlorthalidone, indapamide, or metolazone. In certain embodiments, the diuretic is a potassium sparing diuretic.

In certain embodiments, the additional therapeutic agent is an ACE inhibitor. In certain embodiments, the ACE inhibitor is selected from the group consisting of benazepril, captopril, enalapril, fosinopril, lisinopril, moexipril, perindopril, quinapril, ramipril and trandolapril.

In certain embodiments, the additional therapeutic agent is an angiotensin receptor blocker (“ARB”). In certain embodiments, the ARB is eprosartan, olmesartan, valsartan, telmisartan, losartan, azilsartan medoxomil, candesartan, or irbesartan.

In certain embodiments, the additional therapeutic agent is a β-blocker, a calcium antagonist, or a mineralocorticoid receptor antagonist.

In certain embodiments, the additional therapeutic agent is a natriuretic peptide, such as a B-type natriuretic peptide or N-terminal pro-B-type natriuretic peptide.

In certain embodiments, the additional therapeutic agent is an adenosine antagonist, such as rolofylline.

Specific treatment will be determined on a case-by-case basis by the attending physician.

### Nitroxyl Donors

In certain embodiments, the additional therapeutic agent is a nitroxyl donor, and the method further comprises administering a therapeutically effective amount of the nitroxyl donor.

In particular embodiments, the nitroxyl donor is selected from the compounds described in one or more of U.S. Pat. Nos. 9,499,511; 9,487,498; 9,464,061; 9,458,127; 9,221,780; 9,181,213; 9,156,804; 9,115,064; 9,018,411; 8,987,326; RE45,314; 8,674,132; 8,227,639; and 8,030,356, the disclosures of which are incorporated herein by reference in their entireties.

In selected embodiments, the nitroxyl donor is selected from the compounds described in U.S. Pat. No. RE45,314. In specific embodiments, the nitroxyl donor is selected from the compounds described in U.S. Pat. No. 9,156,804.

### Sodium-Free Chloride Supplementation

In certain embodiments, the additional therapeutic agent is a sodium free chloride salt. In certain embodiments, the agent is lysine chloride.

### Methods of Improving Treatment of Heart Failure

In another aspect, methods are provided for improving treatment of heart failure by discontinuing therapy that is ineffective, thereby reducing side effects and reducing cost without loss of treatment efficacy. The methods comprise discontinuing administration of an IL-6 antagonist to a patient with heart failure, wherein the patient has been determined to be homozygous for the TMPRSS6 rs855791 minor allele. In one series of embodiments, the patient has previously been determined to be homozygous for the TMPRSS6 rs855791 minor allele. In another series of embodiments, the method further comprises the earlier step of determining that the patient is homozygous for the TMPRSS6 rs855791 minor allele.

In certain embodiments, the patient has elevated pre-treatment urine levels of IL-6. In certain embodiments, the patient has elevated pre-treatment plasma levels of IL-6. In certain embodiments, the patient has elevated pre-treatment urine and elevated pre-treatment plasma levels of IL-6.

In particular embodiments, the patient has cardiorenal syndrome.

### Diagnostic, Prognostic, and Treatment Guidance Methods

In another aspect, methods are provided for determining if a subject would benefit from IL-6 antagonist treatment for heart failure. The method comprises measuring a level of IL-6 in a urine sample or plasma sample from the subject, comparing the measured level of IL-6 to a predetermined reference level, and determining whether or not the measured level of IL-6 is greater than the corresponding reference level, wherein when the measured level of IL-6 is greater than the corresponding reference level, IL-6 antagonist treatment is recommended.

IL-6 may be measured in plasma or in urine according to the methods set forth in Section 6.3.2.3 above. Reference levels for IL-6 in urine and plasma may be determined by measuring IL-6 levels in a reference population. A person of skill in the art is able to determine a reference level for the level of a biomarker in a population based on clinical experience and common levels of the biomarker in samples from the population.

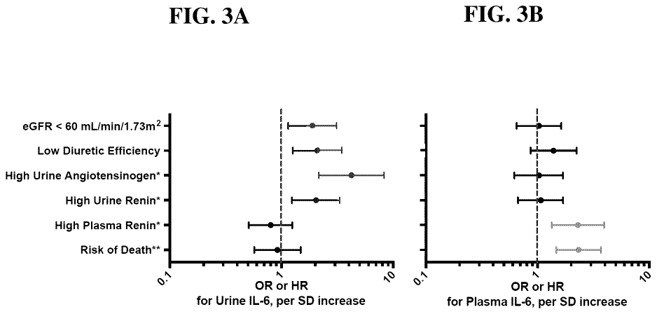

In further aspects, methods are provided for determining if a subject is in need of IL-6 antagonist treatment for impaired glomerular filtration, low diuretic efficiency, high urine angiotensin, high plasma renin, or is at risk of mortality due to cardiorenal syndrome using the above described method. As shown in FIG. 3 and discussed in Example 1 hereinbelow, high levels of IL-6 in urine or plasma are associated with these parameters.

### Kits

In a further aspect, a kit is provided. In general, kits will comprise detection reagents that are suitable for detecting the presence of biomarkers of interest and with instructions for use in accordance with the methods of the invention. The kit may comprise antibodies or other immunohistochemical reagents capable of binding to IL-6. The kit may contain capture and detection antibodies suitable for performing an ELISA for measuring IL-6 in urine or plasma. In certain embodiments the kit may contain tools and reagents for preparing urine and plasma samples for the ELISA or for indexing IL-6 in urine to the concentration of another biomarker, in certain embodiments IL-6 is indexed against creatinine.

### Further Embodiments

Further embodiments are provided in the following numbered embodiments.
1. A method of treating heart failure, comprising:
   administering a therapeutically effective amount of an IL-6 antagonist to a patient with heart failure,
   wherein the patient has been determined to have at least one copy of the TMPRSS6 rs855791 major allele.
2. The method of embodiment 1, wherein the patient has previously been determined to have at least one copy of the TMPRSS6 rs855791 major allele.
3. The method of embodiment 1, further comprising the earlier step of: determining that the patient has at least one copy of the TMPRSS6 rs855791 major allele.
4. The method of any one of embodiments 1-3, wherein the patient has elevated pre-treatment urine levels of IL-6.
5. The method of any one of embodiments 1-4, wherein the patient has elevated pre-treatment plasma levels of IL-6.
6. The method of any one of embodiments 1-5, wherein the patient has acute heart failure.
7. The method of any one of embodiments 1-5, wherein the patient has chronic heart failure.
8. The method of any one of embodiments 1-7, wherein the patient has cardiorenal syndrome.
9. The method of embodiment 8, wherein the patient has cardiorenal syndrome type 4.
10. The method of any one of embodiments 1-9, wherein the patient has diuretic resistant heart failure.
11. The method of embodiment 10, wherein the patient's diuretic efficiency is less than 95.
12. The method of embodiment 11, wherein the patient's diuretic efficiency is less than 90.
13. The method of embodiment 12, wherein the patient's diuretic efficiency is less than 85.
14. The method of embodiment 13, wherein the patient's diuretic efficiency is less than 80.
15. The method of embodiment 14, wherein the patient's diuretic efficiency is less than 75.
16. The method of embodiment 15, wherein the patient's diuretic efficiency is less than 70.
17. The method of embodiment 16, wherein the patient's diuretic efficiency is less than 65.
18. The method of any one of embodiments 1-17, wherein the IL-6 antagonist is an anti-IL-6 antibody, or antigen-binding fragment or derivative thereof.
19. The method of embodiment 18, wherein the anti-IL-6 antibody or antigen-binding fragment or derivative has a K_{D} for binding human IL-6 of less than 100 nM.
20. The method of embodiment 19, wherein the antibody or antigen-binding fragment or derivative has a K_{D} for binding human IL-6 of less than 50 nM.
21. The method of embodiment 20, wherein the antibody or antigen-binding fragment or derivative has a K_{D} for binding human IL-6 of less than 10 nM.
22. The method of embodiment 21, wherein the antibody or antigen-binding fragment or derivative has a K_{D} for binding human IL-6 of less than 1 nM.
23. The method of any one of embodiments 18-22, wherein the anti-IL-6 antibody or antigen-binding fragment or derivative has an elimination half-life following intravenous administration of at least 7 days.
24. The method of embodiment 23, wherein the anti-IL-6 antibody or antigen-binding fragment or derivative has an elimination half-life following intravenous administration of at least 14 days.
25. The method of embodiment 24, wherein the anti-IL-6 antibody or antigen-binding fragment or derivative has an elimination half-life following intravenous administration of at least 21 days.
26. The method of embodiment 25, wherein the anti-IL-6 antibody or antigen-binding fragment or derivative has an elimination half-life following intravenous administration of at least 30 days.
27. The method of any one of embodiments 18-26, wherein the IL-6 antagonist is a full-length monoclonal anti-IL-6 antibody.
28. The method of embodiment 27, wherein the antibody is an IgG1 or IgG4 antibody.
29. The method of embodiment 28, wherein the antibody is an IgG1 antibody.
30. The method of any one of embodiments 18-29, wherein the anti-IL-6 antibody or antigen-binding fragment or derivative is fully human.
31. The method of any one of embodiments 18-29, wherein the anti-IL-6 antibody or antigen-binding fragment or derivative is humanized.
32. The method of any one of embodiments 18-31, wherein the anti-IL-6 antibody or antigen-binding fragment or derivative comprises all six variable region CDRs of MED5117.
33. The method of embodiment 32, wherein the antibody comprises the VH and VL of MED5117.
34. The method of embodiment 33, wherein the antibody is MED5117.
35. The method of any one of embodiments 18-31, wherein the anti-IL-6 antibody or antigen-binding fragment or derivative comprises all six variable region CDRs of an antibody selected from the group consisting of siltuximab, gerilimzumab, sirukumab, clazakizumab, olokizumab, elsilimomab, VX30 (VOP-R003; Vaccinex), EB-007 (EBI-029; Eleven Bio), ARGX-109 (ArGEN-X), FM101 (Femta Pharmaceuticals, Lonza) and ALD518/BMS-945429 (Alder Biopharmaceuticals, Bristol-Myers Squibb).
36. The method of embodiment 35, wherein the anti-IL-6 antibody or antigen-binding fragment or derivative comprises the heavy chain V region and light chain V region from an antibody selected from the group consisting of siltuximab, gerilimzumab, sirukumab, clazakizumab, olokizumab, VX30 (VOP-R003; Vaccinex), EB-007 (EBI-029; Eleven Bio), ARGX-109 (ArGEN-X), FM101 (Femta Pharmaceuticals, Lonza) and ALD518/BMS-945429 (Alder Biopharmaceuticals, Bristol-Myers Squibb).
37. The method of embodiment 36, wherein the anti-IL-6 antibody or antigen-binding fragment or derivative is an antibody selected from the group consisting of siltuximab, gerilimzumab, sirukumab, clazakizumab, olokizumab, VX30 (VOP-R003; Vaccinex), EB-007 (EBI-029; Eleven Bio), ARGX-109 (ArGEN-X), FM101 (Femta Pharmaceuticals, Lonza) and ALD518/BMS-945429 (Alder Biopharmaceuticals, Bristol-Myers Squibb).
38. The method of any one of embodiments 18-26, wherein the IL-6 antagonist is a single domain antibody, a V_{HH} Nanobody, an Fab, or a scFv.
39. The method of any one of embodiments 1-17, wherein the IL-6 antagonist is an anti-IL-6R antibody, or antigen-binding fragment or derivative thereof.
40. The method of embodiment 39, wherein the anti-IL-6R antibody, antigen-binding fragment, or derivative comprises the 6 CDRs of tocilizumab.
41. The method of embodiment 39, wherein the anti-IL-6R antibody, antigen-binding fragment, or derivative comprises the 6 CDRs of vobarilizumab.
42. The method of any one of embodiments 1-17, wherein the IL-6 antagonist is a JAK inhibitor.
43. The method of embodiment 42, wherein the JAK inhibitor is selected from the group consisting of tofacitinib (Xeljanz), decernotinib, ruxolitinib, upadacitinib, baricitinib, filgotinib, lestaurtinib, pacritinib, peficitinib, INCB-039110, ABT-494, INCB-047986 and AC-410.
44. The method of any one of embodiments 1-17, wherein the IL-6 antagonist is a STAT3 inhibitor.
45. The method of any one of embodiments 18-41, wherein the IL-6 antagonist is administered parenterally.
46. The method of embodiment 45, wherein the IL-6 antagonist is administered subcutaneously.
47. The method of any one of embodiments 42 or 43, wherein the IL-6 antagonist is administered orally.
48. The method of any one of embodiments 1-47, wherein the IL-6 antagonist is administered at a dose, on a schedule, and for a period sufficient to increase diuretic efficiency.
49. The method of embodiment 48, wherein the IL-6 antagonist is administered at a dose, on a schedule, and for a period sufficient to increase diuretic efficiency to normal levels.
50. The method of any one of embodiments 1-49, wherein the IL-6 antagonist is administered at a dose, on a schedule, and for a period sufficient to increase eGFR.
51. The method of embodiment 50, wherein the IL-6 antagonist is administered at a dose, on a schedule, and for a period sufficient to increase eGFR to normal levels.
52. The method of any one of embodiments 1-51, further comprising the subsequent step of determining the level of IL-6 in urine.
53. The method of any one of embodiments 1-51, further comprising the subsequent step of determining the level of IL-6 in plasma.
54. The method of any one of embodiments 1-51, further comprising the subsequent step of determining the level of IL-6 in urine and in plasma.
55. The method of any one of embodiments 52-54, further comprising a final step of adjusting the dose of IL-6 antagonist for subsequent administration based on IL-6 level determined in the immediately preceding step.
56. A method of determining if a subject requires treatment for cardiorenal syndrome, the method comprising:
   a) measuring a level of IL-6 in a urine or plasma sample from the subject,
   b) comparing the measured level of IL-6 to a predetermined reference level,
   c) determining whether or not the measured level of IL-6 is greater than the corresponding reference level,
   wherein when the measured level of IL-6 is greater than the corresponding reference level, the subject has cardiorenal syndrome, and treatment is recommended.
57. A method of determining if a subject requires treatment for impaired glomerular filtration, comprising:
   a) measuring a level of IL-6 in a urine sample from the subject,
   b) comparing the measured level of IL-6 to a predetermined reference level,
   c) determining whether or not the measured level of IL-6 is greater than the corresponding reference level,
   wherein when the measured level of IL-6 is greater than the corresponding reference level, the subject has impaired glomerular filtration, and treatment is recommended.
58. A method of determining if a subject requires treatment for low diuretic efficiency, comprising:
   a) measuring a level of IL-6 in a urine sample from the subject,
   b) comparing the measured level of IL-6 to a predetermined reference level,
   c) determining whether or not the measured level of IL-6 is greater than the corresponding reference level,
   wherein when the measured level of IL-6 is greater than the corresponding reference level, the subject has low diuretic efficiency, and treatment is recommended.
59. A method of determining if a subject requires treatment for high urine angiotensin, comprising:
   a) measuring a level of IL-6 in a urine sample from the subject,
   b) comparing the measured level of IL-6 to a predetermined reference level,
   c) determining whether or not the measured level of IL-6 is greater than the corresponding reference level,
   wherein when the measured level of IL-6 is greater than the corresponding reference level, the subject has high urine angiotensin, and treatment is recommended.
60. A method of determining if a subject requires treatment for high plasma renin, comprising:
   a) measuring a level of IL-6 in a plasma sample from the patient,
   b) comparing the measured level of IL-6 to a predetermined reference level,
   c) determining whether or not the measured level of IL-6 is greater than the corresponding reference level,
   wherein when the measured level of IL-6 is greater than the corresponding reference level, the subject has high plasma renin, and treatment is recommended.
61. A method of determining if a subject is at risk of mortality due to cardiorenal syndrome and requires treatment, comprising:
   a) measuring a level of IL-6 in a plasma sample from the patient,
   b) comparing the measured level of IL-6 to a predetermined reference level,
   c) determining whether or not the measured level of IL-6 is greater than the corresponding reference level,
   wherein when the measured level of IL-6 is greater than the corresponding reference level, the subject is at risk of mortality due to cardiorenal syndrome, and treatment is recommended.
62. The method of any one of embodiments 56-61, wherein the patient is a heart failure patient.
63. The method of any of embodiments 56-62, wherein the level of IL-6 is measured using an enzyme-linked immunosorbent assay (ELISA).
64. A method of treating cardiorenal syndrome in a patient comprising:
   a) measuring a level of IL-6 in a urine sample or a plasma sample from the patient,
   b) comparing the measured level of IL-6 to a predetermined reference level,
   c) determining whether or not the measured level of IL-6 is greater than the corresponding reference level
   wherein when the measured level of IL-6 is greater than the corresponding reference level, treatment for cardiorenal syndrome is administered.
65. The method of embodiment 64, wherein the patient is a heart failure patient.
66. The method of embodiment 64 or embodiment 65, wherein the level of IL-6 is measured using an enzyme-linked immunosorbent assay (ELISA).
67. The method of any one of embodiments 64-66, wherein the treatment administered for cardiorenal syndrome comprises administering one or more of at least one diuretic, angiotensin-converting-enzyme inhibitor, angiotensin receptor blocker, natriuretic peptide, adenosine antagonist, an IL-6 antagonist, or any combination thereof.
68. The method of embodiment 67, wherein treating cardiorenal syndrome comprises administering at least one anti-IL-6 antibody or anti-IL-6R antibody.
69. A kit comprising reagents for an assay for measuring a level of IL-6 and written instructions, the written instructions comprising:
   a) measuring a level of IL-6 in a urine or plasma sample from the subject,
   b) comparing the measured level of IL-6 to a predetermined reference level,
   c) determining whether or not the measured level of IL-6 is greater than the corresponding reference level,
   wherein when the measured level of IL-6 is greater than the corresponding reference level, the subject has cardiorenal syndrome, and treatment is recommended.
70. The kit of embodiment 69, wherein the instructions direct the kit for use on a heart failure patient.
71. The kit of embodiment 69 or 70, wherein the kit contains reagents for measuring the level of IL 6 using an enzyme-linked immunosorbent assay (ELISA).

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, practice the claimed methods of the present invention. The following working examples therefore, specifically point out the preferred embodiments of the present invention, and are not to be construed as limiting in any way the remainder of the disclosure.

The materials and methods employed in the experiments disclosed herein are now described.

### Example 1: Urine and Plasma IL-6 Associations in Heart Failure Patients

Methods. Consecutive heart failure (“HF”) patients receiving high dose diuretic therapy at the Yale Transitional Care Clinic (YTCC) were enrolled in the prospective observational study.

The YTCC is an outpatient clinic with a focus on diuretic and fluid status management. Patients present for 4-8 hours of treatment, during which they receive 1-3 doses of loop diuretic. The dosing protocol is determined by patient fluid status; the choice of oral (PO) torsemide or intravenous (IV) bumetanide is at the discretion of the treating physician. During the treatment period all urine produced is saved in a cumulative collection container and sent to the clinical laboratory for electrolyte measurements at the conclusion of the visit. A cumulative urine collection is conducted during the treatment period. Additional spot urine samples are saved both before and one hour after diuretic administration.

Assays. A Randox RxDaytona™ automated clinical chemistry analyzer was used to measure urine and serum electrolytes using ion selective electrodes. Urea, creatinine, bicarbonate and cystatin C were measured using Randox reagents per the manufacturer's instructions (Randox Laboratories™, UK). Concentrations of interleukin 10 and IL-6 were measured using the MesoScale Discovery (MSD) platform (Meso Scale Diagnostics™, Gaithersburg, MD, USA). Levels of amino terminal pro B-type natriuretic peptide (NT-proBNP) were measured at the Yale clinical laboratory on a Roche Elecsys 120 analyzer (Roche Diagnostics, Indianapolis, USA). Plasma renin activity (PRA), angiotensinogen and active renin were measured using commercially available competitive ELISA kits from ALPCO™ per manufacturer's instructions (ALPCO™, Salem, NH, USA). Total renin was also analyzed using commercially available ELISA kits (R&D Systems™, Minneapolis, USA). The total renin immunoassay kit from R&D Systems™ recognizes both active renin and prorenin. The assay's mean detectable limit is 4.43 pg/ml for total renin and 0.81 pg/ml for active renin. Liquid chromatography mass spectrometry was used to measure levels of bumetanide and torsemide in urine. Ultra-high performance liquid chromatography was performed on the Agilent Infinity 1290 UPLC System™. Chromatographic separation was achieved on the Zorbax Bonus RP™ 2.1×50 mm 1.8μ column with a flow rate of 0.6 ml/min. The mobile phase contained of 0.1% Formic acid (Buffer A) and 80% acetonitrile in 0.1% Formic acid (Buffer B). Mass spectrometry was performed on Agilent Q-TOF System™ (Agilent™, Santa Clara, CA, USA) in positive ion mode.

Calculations and Definitions. eGFR was calculated using the Chronic Kidney Disease Epidemiology Collaboration (CKD-EPI) formula. Doses of loop diuretics were converted to furosemide equivalents with 1 mg bumetanide=20 mg torsemide=40 mg intravenous furosemide=80 mg oral furosemide. As published previously, diuretic efficiency was defined as the increase in urinary sodium output over the treatment period per each doubling of the loop diuretic dose, centered on 40 mg of furosemide equivalents; this scale was chosen to account for the sigmoidal dose-response curve of these drugs. Urinary diuretic excretion was calculated by multiplying the concentration of diuretic in the urine by the volume of urine produced in the first 3 hours after diuretic administration; this quantity of estimated diuretic was then normalized to the administered diuretic dose in furosemide equivalents, taking into account the published urinary clearance of the particular diuretic received by the patient (bumetanide or torsemide). Fractional excretions of sodium and potassium were calculated using the formula: Fractional excretion of X (FEX)=[X]Urine*[Creatinine]Serum/([X] Serum or plasma*[Creatinine]Urine). Urine proteins including renin, angiotensinogen and IL-6 were indexed to urinary creatinine. A low urine IL-6 was defined as a value less than the median of 14.2 pg/g of urinary creatinine. A low plasma IL-6 was defined as a value less than the median of 2.0 pg/mL. High levels of urine and plasma renin and angiotensinogen were defined as values greater than or equal to the cohort medians of these variables.

Statistical Analysis. Values reported are mean+/−SD, median (quartile 1-quartile 3) and percentage. Correlations between continuous variables are Spearman's rho with the exception of adjusted correlations. Pearson's chi-squared test was used to compare categorical variables between groups. To compare continuous variables between groups, either Student's t-test or the Wilcoxon Rank Sum test was used. A log transformation was applied to skewed variables including plasma IL-6, urine IL-6 and NT-pro-BNP before entering them into multivariable models and partial correlation analyses. Logistic regression was used to evaluate association between the odds of low diuretic efficiency, an eGFR<60 mL/min/1.73 m², or high levels of urine or plasma neurohormonal parameters with plasma and urine levels of IL-6, both on a univariate level and with adjustment for plasma or urine IL-6 and/or eGFR. Cox proportional hazards modeling was used to evaluate time-to-event associations with all-cause mortality. Statistical analysis was performed with IBM SPSS Statistics version 23 (IBM Corp., Armonk, NY) and Stata version 13 (StataCorp™, College Station, TX). Statistical significance was defined as 2-tailed p<0.05 for all analyses.

The baseline characteristics of our population are described in Table 2, below. In this subset, 98 patients underwent determination of IL-6 levels in blood and urine. The median (IQR) pre-diuretic level of urine IL-6 was 14.2 pg/g of creatinine (5.6-36.2 pg/g) whereas the median level of plasma IL-6 was 2.0 pg/mL (1.2-3.9 pg/mL). Plasma and urine IL-6 levels were only modestly correlated (r=0.40, p<0.00l). Notably, those with lower than median levels of urine IL-6 tended to be younger, Caucasian, more often on angiotensin receptor blocker (ARB) or angiotensin converting enzyme (ACE-I) therapy, more likely to have heart failure with reduced ejection fraction (HFrEF), a higher GFR and substantially lower plasma levels of NT-proBNP levels. The profile of patients defined by a lower than median plasma IL-6 was somewhat different, but largely reflected the same trends.

TABLE 2
Baseline Characteristics

| | Overall | Low Urine | High Urine | | Low Plasma | High Plasma | |
|---|---|---|---|---|---|---|---|
| | Cohort | IL-6 | IL-6 | | IL-6 | IL-6 | |
| | (n = 98) | (n = 49) | (n = 49) | p-value | (n = 49) | (n = 49) | p-value |
| Demographics | | | | | | | |
| Age | 67.9 ± 13.5 | 62.1 ± 13.2 | 73.8 ± 11.2 | <0.001* | 65.8 ± 15.1 | 70.1 ± 11.5 | 0.12 |
| Black race, % | 36 | 57 | 15 | <0.001* | 47 | 26 | 0.04* |
| Male sex, % | 44 | 49 | 39 | 0.31 | 49 | 39 | 0.31 |
| Past Medical History, % | | | | | | | |
| Hypertension | 91 | 94 | 88 | 0.29 | 96 | 86 | 0.08 |
| Diabetes | 47 | 55 | 39 | 0.11 | 37 | 57 | 0.04* |
| Gout | 13 | 10 | 16 | 0.37 | 10 | 16 | 0.37 |
| Ischemic etiology | 30 | 33 | 27 | 0.46 | 33 | 27 | 0.46 |
| Post-discharge follow-up visit | 63 | 71 | 55 | 0.09 | 61 | 65 | 0.68 |
| Baseline Medications, % | | | | | | | |
| ACEI or ARB | 55 | 65 | 45 | 0.04* | 67 | 43 | 0.02* |
| Beta-blocker | 77 | 82 | 71 | 0.23 | 82 | 71 | 0.23 |
| Thiazide-type diuretic | 14 | 12 | 16 | 0.56 | 6 | 22 | 0.02* |
| MRA | 33 | 37 | 29 | 0.39 | 31 | 35 | 0.67 |
| Digoxin | 10 | 12 | 8 | 0.50 | 8 | 12 | 0.50 |
| Home loop diuretic dose | 80 | 80 | 100 | 0.60 | 80 | 160 | 0.002* |
| (mg furosemide equivalents) | (60-160) | (60-160) | (80-160) | | (40-120) | (80-200) | |
| Physical Exam | | | | | | | |
| Weight (kg) | 98.2 ± 33.4 | 106.0 ± 37.9 | 90.4 ± 26.4 | 0.02* | 98.4 ± 31.5 | 98.0 ± 35.5 | 0.95 |
| BMI | 34.8 ± 10.8 | 36.7 ± 11.3 | 32.5 ± 9.9 | 0.09 | 33.5 ± 8.4 | 36.2 ± 13.0 | 0.28 |
| Systolic blood pressure (mmHg) | 122.6 ± 18.9 | 120.6 ± 19.1 | 124.5 ± 18.7 | 0.32 | 123.4 ± 18.9 | 121.7 ± 19.1 | 0.65 |
| Heart rate (beats per minute): | 75.9 ± 13.2 | 75.6 ± 13.2 | 76.2 ± 13.4 | 0.82 | 74.9 ± 13.3 | 76.8 ± 13.2 | 0.48 |
| Echocardiogram | | | | | | | |
| Left ventricular | 45 | 35 | 51 | 0.03* | 39 | 45 | 0.56 |
| ejection fraction (%) | (27-56) | (25-50) | (31-59) | | (29-54) | (26-57) | |
| Ejection fraction on ≥40% | 53% | 43% | 63% | 0.04* | 49% | 57% | 0.42 |
| Laboratory Values | | | | | | | |
| Sodium (mmol/L) | 136 | 137 | 139 | 0.06 | 138 | 136 | 0.12 |
| | (134-140) | (134-138) | (134-141) | | (135-140) | (132-140.0) | |
| Chloride (mmol/L) | 96 | 100 | 98 | 0.50 | 101 | 96 | <0.001* |
| | (95-102) | (96-102) | (95-102) | | (98-103) | (91-101) | |
| Hypochloremia, % | 34 | 27 | 42 | 0.13 | 14 | 55 | <0.001* |
| Potassium (mmol/L) | 4.1 | 4.2 | 4.1 | 0.053 | 4.1 | 4.1 | 0.64 |
| | (3.7-4.5) | (3.8-4.6) | (3.6-4.3) | | (3.7-4.5) | (3.7-4.4) | |
| Bicarbonate (mmol/L) | 23.9 | 23.1 | 24.9 | 0.03* | 23.1 | 25.0 | 0.03* |
| | (21.7-26.4) | (21.6-25.3) | (22.2-27.9) | | (21.5-25.2) | (22.6-28.0) | |
| Blood urea nitrogen (mg/dL) | 29.0 | 27.0 | 32.0 | 0.10 | 27.0 | 36.0 | 0.04* |
| | (20.0-47.0) | (17.5-44.0) | (24.0-48.0) | | (19.0-39.0) | (23.0-65.0) | |
| Serum creatinine (mg/dL) | 1.5 ± 0.8 | 1.4 ± 0.6 | 1.7 ± 0.9 | 0.046* | 1.3 ± 0.5 | 1.8 ± 1.0 | 0.007* |
| BUN/Creatinine ratio | 24.0 ± 8.3 | 24.1 ± 8.1 | 23.9 ± 8.5 | 0.92 | 23.6 ± 8.0 | 24.5 ± 8.5 | 0.58 |
| Estimated glomerular | 54.9 ± 28.2 | 64.3 ± 30.1 | 45.7 ± 22.8 | <0.001* | 61.4 ± 27.2 | 48.3 ± 27.8 | 0.02* |
| filtration rate (eGFR) | | | | | | | |
| (mL/min/1.73 m²) | | | | | | | |
| eGFR <60 mL/min/1.73 m² | 61% | 48% | 73% | 0.01* | 55% | 67% | 0.24 |
| Albumin (g/dL) | 3.8 ± 0.4 | 3.9 ± 0.4 | 3.8 ± 0.4 | 0.045* | 4.0 ± 0.4 | 3.7 ± 0.4 | <0.001* |
| Hemoglobin (g/dL) | 12.2 ± 2.3 | 12.7 ± 2.2 | 11.7 ± 2.3 | 0.04* | 12.8 ± 2.0 | 11.5 ± 2.4 | 0.004* |
| Diuretic Parameters | | | | | | | |
| Diuretic dose administered | 140 | 80 | 160 | 0.001* | 80 | 160 | <0.001* |
| (mg furosemide equivalents) | (40-240) | (40-160) | (80-280) | | (40-160) | (80-320) | |
| Diuretic administered, % | | | | | | | |
| IV Bumetanide: | 45 | 27 | 63 | <0.001* | 33 | 57 | 0.02* |
| Torsemide: | 55 | 73 | 37 | | 67 | 43 | |

ACE-I = angiotensin-converting enzyme inhibitor.
ARB = angiotensin receptor blocker.
MRA = mineralocorticoid receptor antagonist.
IL = interleukin.
BUN = blood urea nitrogen.
BNP = brain natriuretic peptide.
BMI = body mass index.
*p < 0.05.

TABLE 3
Plasma and Urine Biomarkers at Baseline

| | Overall | Low Urine | High Urine | | Low Plasma | High Plasma | |
|---|---|---|---|---|---|---|---|
| | Cohort | IL-6 | IL-6 | | IL-6 | IL-6 | |
| | (n = 98) | (n = 49) | (n = 49) | p-value | (n = 49) | (n= 49) | p-value |
| Plasma Biomarkers | | | | | | | |
| IL-6 (pg/mL) | 2.0 | 1.4 | 2.8 | <0.001* | 1.2 | 3.9 | <0.001* |
| | (1.2-3.9) | (0.9-2.9) | (1.5-6.1) | | (0.9-1.4) | (2.9-6.8) | |
| IL-10 (pg/mL) | 0.3 | 0.3 | 0.5 | 0.001* | 0.3 | 0.5 | 0.01* |
| | (0.2-0.7) | (0.2-0.4) | (0.3-0.8) | | (0.2-0.5) | (0.3-0.8) | |
| IL-10/IL-6 ratio | 0.2 | 0.2 | 0.2 | 0.73 | 0.3 | 0.1 | <0.001* |
| | (0.1-0.4) | (0.1-0.3) | (0.1-0.4) | | (0.2-0.6) | (0.1-0.2) | |
| Total renin (pg/mL) | 1167.7 | 1169.2 | 1166.3 | 0.97 | 909.6 | 1784.4 | <0.001* |
| | (734.7-2759.7) | (734.7-3013.5) | (816.0-2390.9) | | (595.2-1517.9) | (921.6-3868.7) | |
| Active renin (pg/mL) | 36.8 | 79.0 | 28.1 | 0.38 | 13.0 | 49.1 | 0.07 |
| | (8.4-162.8) | (7.7-184.2) | (12.0-139.4) | | (4.4-160.7) | (15.7-169.2) | |
| Angiotensinogen (ng/mL) | 167.9 | 193.9 | 160.3 | 0.81 | 178.8 | 167.2 | 0.50 |
| | (54.1, 445.3) | (48.2, 518.1) | (58.3, 430.2) | | (31.6, 481.2) | (75.5, 445.3) | |
| NT pro-BNP (pg/mL) | 1825.0 | 840.0 | 3115.0 | <0.001* | 1440.0 | 3390.0 | 0.01* |
| | (681.0-5030.0) | (256.0-3115.0) | (1440.0-6070.0) | | (412.0-3010.0) | (792.0-6410.0) | |
| Urine Biomarkers | | | | | | | |
| IL-6 (pg/g of creatinine) | 14.2 | 5.6 | 36.2 | <0.001* | 7.0 | 21.2 | <0.001* |
| | (5.6-36.2) | (2.5-7.7) | (20.7-68.9) | | (3.2-19.1) | (9.2-45.3) | |
| IL-6 (pg/mL) | 1.2 | 0.5 | 2.5 | <0.001* | 0.8 | 1.7 | 0.03* |
| | (0.5-2.6) | (0.2-0.7) | (1.6-4.0) | | (0.4-1.9) | (0.6-3.2) | |
| Renin (pg/mg of | 0.3 | 0.2 | 0.6 | <0.001* | 0.2 | 0.5 | <0.001* |
| creatinine) | (0.1-0.8) | (0.1-0.5) | (0.3-1.2) | | (0.1-0.6) | (0.2-1.5) | |
| Renin (pg/mL) | 28.2 | 19.9 | 37.0 | 0.01* | 23.7 | 35.9 | 0.06 |
| | (10.2-88.3) | (7.5-53.1) | (13.4-109.6) | | (7.5-53.1) | (12.3-109.6) | |
| Angiotensinogen | 18.7 | 6.2 | 123.5 | <0.001* | 7.3 | 33.7 | 0.002* |
| (pg/mg creatinine) | (5.4-126.3) | (3.3-19.0) | (17.3-549.7) | | (3.7-39.7) | (12.3-318.9) | |
| Angiotensinogen (pg/mL) | 1530 | 966 | 5850 | <0.001* | 1105 | 2153 | 0.02* |
| | (624-10941) | (231-1567) | (1518-53865) | | (463-5801) | (966-23952) | |
| FE_{Na} (%) | 0.5 | 0.4 | 0.5 | 0.22 | 0.4 | 0.7 | 0.02* |
| | (0.2-1.3) | (0.2-1.2) | (0.2-1.4) | | (0.1-1.0) | (0.3-1.8) | |
| FE_{K} (%) | 18.4 | 15.5 | 21.6 | 0.02* | 15.8 | 24.5 | 0.001* |
| | (11.7-39.3) | (9.9-32.5) | (15.2-44.6) | | (9.6-24.5) | (13.9-50.3) | |
| FE_{Urea} (%) | 63.6 | 59.5 | 65.5 | 0.62 | 66.7 | 58.2 | 0.20 |
| | (44.8-81.3) | (44.4-80.5) | (44.9-81.6) | | (47.5-82.8) | (44.6-76.0) | |

IL = interleukin.
NT pro-BNP = N-terminal pro b-type natriuretic peptide.
FENa = fractional excretion of sodium.
FEK = fractional excretion of potassium.
FEUrea = fractional excretion of urea.

### Kidney Function and IL-6

As shown in Table 2, eGFR was lower in patients with high urine or plasma IL-6, but this association was only significant between eGFR and urine IL-6 (p=0.01). A correlation between both plasma IL-6 and eGFR (r=−0.26, p=0.0l) as well as urine IL-6 and eGFR (r=−0.38, p<0.00l) was observed. However, on adjustment for urine IL-6, there was no longer a significant association between plasma IL-6 and eGFR (p=0.20), whereas a significant association remained between urine IL-6 and eGFR after adjustment for plasma IL-6 (partial r=−0.32, p=0.002). Similarly, the risk of reduced eGFR as defined by an eGFR<60 mL/min/1.73 m² was increased with higher levels of urine IL-6 (OR=1.9 per SD increase, 95% CI=1.2, 3.1, p=0.006) and not with higher levels of plasma IL-6 (OR=1.3 per SD increase, 95% CI=0.8-2.0, p=0.25).

### Diuretic Response and IL-6

There was an inverse association between diuretic efficiency and both urine IL-6 (r=−0.43, p<0.00l) and plasma IL-6 (r=−0.31, p=0.002; FIG. 3). Odds for a low diuretic efficiency increased with higher levels of either urine IL-6 (OR=2.3 per SD increase, 95% CI 1.4-3.8, p=0.00l) or plasma IL-6 (OR=1.7 per SD increase, 95% CI=1.1-2.7, p=0.02). Upon adjustment for eGFR, only urine IL-6 remained significantly associated with risk of low diuretic efficiency (adjusted OR=1.8 per SD increase, 95% CI 1.1-3.1, p=0.02; FIG. 3). Furthermore, when urine IL-6 and plasma IL-6 were both entered into a logistic regression model, only urine IL-6 remained associated with risk of low diuretic efficiency (adjusted OR=2.1 per SD increase, 95% CI 1.3-3.5, p=0.004) while plasma IL-6 showed no such association (OR=1.4, 95% CI 0.9-2.2, p=0.17).

### Neurohormonal Activation

Plasma IL-6 was associated with higher levels of plasma renin (Table 3 and FIG. 3). Notably, higher levels of plasma IL-6 conferred additional risk of high plasma total renin (OR=1.9 per SD increase, 95% CI 1.2-3.0, p=0.008), and this persisted despite adjustment for use of ACE-I or ARB and urine IL-6 levels (adjusted OR=2.3 per SD increase, 95% CI 1.3-3.9, p=0.003). Urine IL-6 was not associated with increased risk of high plasma renin (OR=1.0, 95% CI 0.7-1.5, p=0.98).

Urine IL-6 was strongly associated with high levels of tissue level RAAS activation as measured by urine angiotensinogen (OR=4.2 per SD increase, 95% CI 2.2-7.9, p<0.00l) and urine renin (OR=2.1 per SD increase, 95% CI 1.3-3.4, p=0.002; FIG. 3). These associations persisted after adjustment for plasma IL-6 levels and ACE-I or ARB use (adjusted OR for high urine angiotensinogen=4.2 per SD increase, 95% CI 2.2-8.3, p<0.001; adjusted OR for high urine renin=2.0 per SD increase, 95% CI 1.2-3.3, p=0.005). Plasma IL-6 did not demonstrate a univariate association with risk of high urine renin (OR=1.3 per SD increase, 95% CI 0.9-2.0, p=0.16). Although a trend toward association with urine angiotensinogen (OR=1.5 per SD increase, 95% CI 0.98-2.3, p=0.06) was observed, it was eliminated with adjustment for urine IL-6 (adjusted OR=1.1, 95% CI 0.7-1.7, p=0.76).

Association with Survival

Over a median follow-up time of 713 days, 32 deaths occurred. Consistent with previous reports, increases in plasma IL-6 were associated with a higher risk of mortality (univariate HR=2.8 per SD increase, 95% CI 2.0-4.0, p<0.001). Plasma IL-6 remained associated with mortality after multivariable adjustment for baseline characteristics including age, race, baseline NT-proBNP levels, use of ACE-I or ARB, home loop diuretic dose and eGFR (adjusted HR=2.3 per SD increase, 95% CI=1.5-3.7, p<0.00l). In contrast, urine IL-6 was not associated the risk of mortality (univariate HR=1.3 per SD increase, 95% CI 0.9-1.8, p=0.15; adjusted HR=1.02, 95% CI=0.6-1.6, p=0.93).

### Summary

Plasma and urine IL-6 levels capture distinctive aspects of the role of IL-6 in cardiorenal disease pathophysiology. Plasma IL-6 levels are associated with global measures of disease severity such as risk of mortality. In contrast, urine IL-6, likely a measure of kidney inflammation, is strongly correlated with multiple measures of cardiorenal syndrome (“CRS”) in these heart failure patients, including diuretic resistance, renin angiotensin aldosterone and system (RAAS) activation, and lower estimated glomerular filtration rate (eGFR).

These data demonstrate that urine IL-6 level is a useful biomarker for renal inflammation, and is particularly useful in assessing renal symptoms in heart failure patients. In addition, serial urine IL-6 levels should prove useful in monitoring and assessing the renal benefit of therapeutic interventions in heart failure.

### Example 2: TMPRSS6 Genotype Stratifies Heart Failure Patients Whose Renal Symptoms are Predicted to be Responsive to IL-6 Antagonists

The data obtained in Example 1 also predict that treatment with an IL-6 antagonist should be effective in reducing renal inflammation in heart failure patients.

However, because infection is often a precipitating cause of acute decompensation in heart failure patients, it is important to limit anti-cytokine and other immunosuppressive therapies to those heart failure patients who are likely to respond with improved renal and/or cardiac function. The cost of chronic IL-6 antagonist therapy also militates for limiting treatment to those heart failure patients who are likely to respond with improved renal and/or cardiac function.

Analysis conducted in Example 1 was expanded to 129 patients. FIG. 1A is a bar graph showing diuretic efficiency (“DE”) by tertiles of urine IL-6, confirming the inverse correlation of urinary IL-6 with diuretic efficiency observed in the 98 patient subset. FIG. 1B is a bar graph showing diuretic efficiency (“DE”) by tertiles of plasma IL-6 in these 129 patients, confirming an inverse correlation of plasma IL-6 with diuretic efficiency (uncorrected by urinary IL-6 levels).

Each patient's genotype at the rs855791 single nucleotide polymorphism (“SNP”) in transmembrane protease serine 6 (“TMPRSS6”) was further assessed. The TMPRSS6 polypeptide, also known as Matriptase-2 (MT2), cleaves hemojuvelin and inhibits bone morphogenetic protein signaling. The rs855791 (G2321A) SNP alters the TMPRSS6 protein sequence: the allele with highest frequency in the human population (the major allele) is 2321G, encoding 736A; the allele with lowest frequency in the human population (minor allele) is 2321A, encoding 736V.

Genomic DNA was isolated from buffy coats using ReliaPrep large volume HT gDNA isolation system on the HSM Instrument (Promega, Madison, USA). The purity of the isolated DNA was assessed by Nanodrop. Genotyping was carried out at the Yale Centre for Genome analysis. Whole genome genotyping was done using Infinium® Exome-24 v1.0 BeadChip Kit from Illumina using standard protocols suggested by the manufacturer (Illumina, Inc., San Diego, CA). The amplification, fragmentation, precipitation, resuspension and hybridization steps were done manually. The arrays were scanned on the Illumina Hiscan instrument. The Illumina HiScan or iScan System scans the BeadChip, using a laser to excite the fluorophore of the single-base extension product on the beads. The scanner records high resolution images of the light emitted from the fluorophores. The Illumina GenomeStudio Genotyping Module, included with the Illumina Infinium Assay system, was used for extracting genotyping data from intensity data files (*.idat files) collected from the Illumina HiScan System. The Infinium Exome-24 v1.0 BeadChip contains over 240,000 putative functional exonic variants selected from over 12,000 individual human exome and whole-genome sequences. The >240,000 markers represent diverse populations, including European, African, Chinese, and Hispanic individuals, and a range of common conditions, such as type 2 diabetes, cancer, metabolic, and psychiatric disorders. Detailed Illumina genotyping protocol is available at http://support.illumina.com (infinium_hts_assay_protocol_user_guide_15045738_a.pdf). The details of the SNPs in this exome chip are available at http://support.illumina.com/downloads/infinium-exome-24-v1-0-product-files.html.

As shown in FIG. 2A, urine levels of IL-6 were inversely correlated with diuretic efficiency only in the patients having at least one copy of the major allele of the TMPRSS6 rs855791 SNP (FIG. 2A, right panel, “AG+GG”); urine levels of IL-6 are not significantly correlated with diuretic efficiency in patients homozygous for the minor allele (FIG. 2A, left panel, “AA”).

As shown in FIG. 2B, plasma levels of IL-6 correlated inversely with diuretic efficiency only in the patients having at least one copy of the major allele of the TMPRSS6 rs855791 SNP (FIG. 2B, right panel, “AG+GG”); plasma levels of IL-6 are not significantly correlated with diuretic efficiency in patients homozygous for the minor allele (FIG. 2B, left panel, “AA”).

These data indicate that treatment of heart failure with an IL-6 antagonist will only improve renal symptoms in heart failure patients having at least one copy of the TMPRSS6 rs855791 major allele.

### Example 3: Correlation of IL-6 with the Expression of NKCC2, ENaC-Beta, and NCC in the Absence or Presence of Ruxolitinib

### Methods

The mouse M1 CCD cell line (American Type Culture Collection (ATCC), Cat #CRL-2038) was maintained the cell culture medium containing equal amount of DMEM (Sigma-Aldrich, Cat #D6046) and Han F12 (Sigma-Aldrich, Cat #11765-047), supplemented with 5% Fetal Bovine Serum (FBS), 1% Penicillin-Streptomycin (Thermo Fisher Scientific, Cat #15140-122), 1% Insulin-Transferrin-Selenium (Thermo Fisher Scientific, Cat #51500-056) and 100 nM Dexamethasone (Sigma-Aldrich, Cat #D4902-100MG).

M1 CCD cells were seeded at 1 million/well in 6-well plate in the cell culture medium, and incubated overnight in a 37° C., 5% CO₂ incubator on Day 0. The cell culture medium was changed to DMEM/F12 serum-free medium on Day 1 and the cells were incubated overnight in a 37° C., 5% CO₂ incubator. On Day 2, the serum-free medium was removed and the cell culture medium was added to each well. Ruxolitinib (Selleckchem, Cat #S1378) was added at the final concentration of 1 μM or 100 μM 10 min before the addition of IL-6 (Sigma-Aldrich, Cat #SRP3096-20UG) at the final concentration of 10 ng/mL, 100 ng/mL, or 500 ng/mL. A control well without Ruxolitinib or IL-6 was included. The cells were treated for 24 hours with IL-6 and/or Ruxolitinib. The cells of each well were washed with once with 1×PBS and collected in 250 μL 1×PIPA buffer (10×, Millipore, Cat #20-188) supplemented with 1% protease inhibitor cocktail (100×, Thermo Fisher Scientific, Cat #78430). The sample were analyzed by immunoblotting using anti-NKCC2 antibody (Millipore, Cat #AB3562P), anti-ENaC-beta antibody (Millipore, Cat #AB3532P), or anti-NCC antibody (Millipore, Cat #AB3553) and the protein expression was quantified. Each experiment was done in triplicate.

### Results

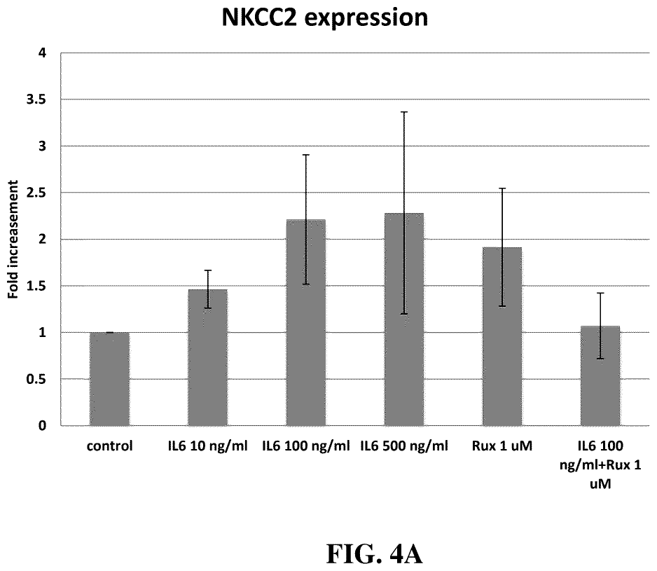

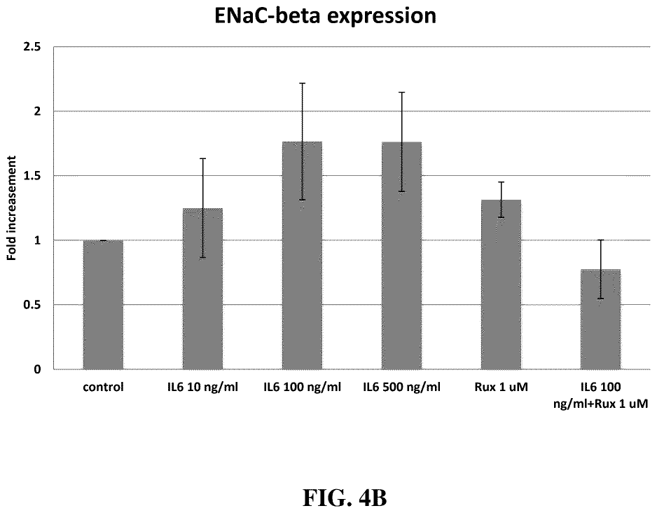

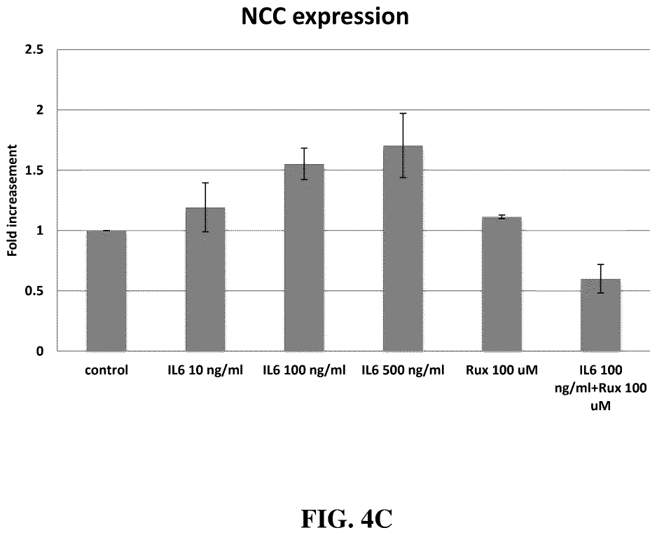

We examined the expression of NKCC2 (Na—K—Cl cotransporter 2), ENaC-beta (epithelial sodium channel, beta subunit), and NCC (sodium-chloride symporter) proteins in mouse M1 CCD cell line after treatment of IL-6 in the absence or presence of a JAK inhibitor, Ruxolitinib. The mouse M1 CCD cells are genotypically analogous to human cells homozygous for the TMPRSS6 rs855791 major allele. As shown in FIGS. 4A, 4B, and 4C, the treatment of IL-6 increased the expression of NKCC2, ENaC-beta, and NCC. Ruxolitinib blocked the effect of IL-6 on the ion transporter proteins.

Increased expression of these ion transporters provides a putative mechanism for IL-6 mediated diuretic resistance.

Because the increased expression is not known to be linked to hepcidin expression, these data suggested that IL-6 antagonism could be effective in treating diuretic resistance even in patients homozygous for the TMPRSS6 rs855791 minor allele.

### Example 4: Correlation of IL-6 with the Expression of NKCC2, ENaC-Beta, and NCC in the Absence or Presence of Spironolactone

### Methods

The mouse M1 CCD cell line (American Type Culture Collection (ATCC), Cat #CRL-2038) was maintained the cell culture medium containing equal amount of DMEM (Sigma-Aldrich, Cat #D6046) and Han F12 (Sigma-Aldrich, Cat #11765-047), supplemented with 5% Fetal Bovine Serum (FBS), 1% Penicillin-Streptomycin (Thermo Fisher Scientific, Cat #15140-122), 1% Insulin-Transferrin-Selenium (Thermo Fisher Scientific, Cat #51500-056) and 100 nM Dexamethasone (Sigma-Aldrich, Cat #D4902-100MG).

M1 CCD cells were seeded at 1 million/well in 6-well plate in the cell culture medium, and incubated overnight in a 37° C., 5% CO₂ incubator on Day 0. The cell culture medium was changed to DMEM/F12 serum-free medium on Day 1 and the cells were incubated overnight in a 37° C., 5% CO₂ incubator. On Day 2, the serum-free medium was removed and the cell culture medium was added to each well. Spironolactone (Selleckchem, Cat #S4054) was added at the final concentration of 1 μM or 100 μM 10 min before the addition of IL-6 (Sigma-Aldrich, Cat #SRP3096-20UG) at the final concentration of 10 ng/mL, 100 ng/mL, or 500 ng/mL. A control well without Spironolactone or IL-6 was included. The cells were treated for 24 hours with IL-6 and/or Spironolactone. The cells of each well were washed with once with 1×PBS and collected in 250 μL 1×PIPA buffer (10×, Millipore, Cat #20-188) supplemented with 1% protease inhibitor cocktail (100×, Thermo Fisher Scientific, Cat #78430). The sample were analyzed by immunoblotting using anti-NKCC2 antibody (Millipore, Cat #AB3562P), anti-ENaC-beta antibody (Millipore, Cat #AB3532P), or anti-NCC antibody (Millipore, Cat #AB3553) and the protein expression was quantified. Each experiment was done in triplicate.

### Results

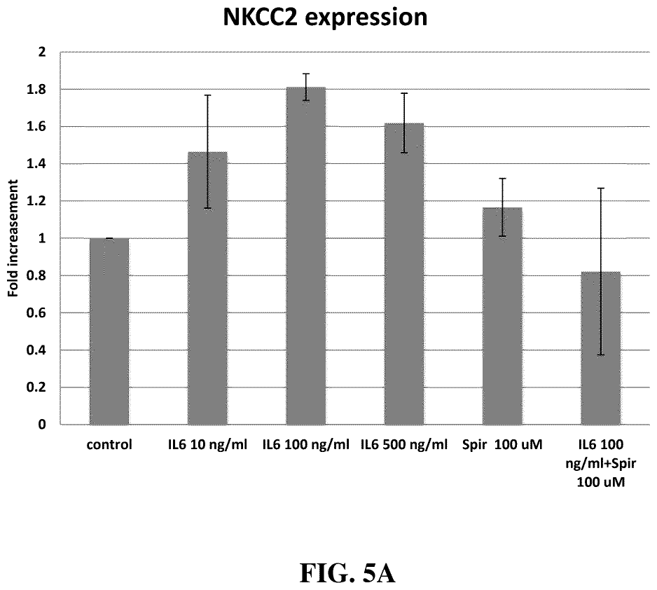

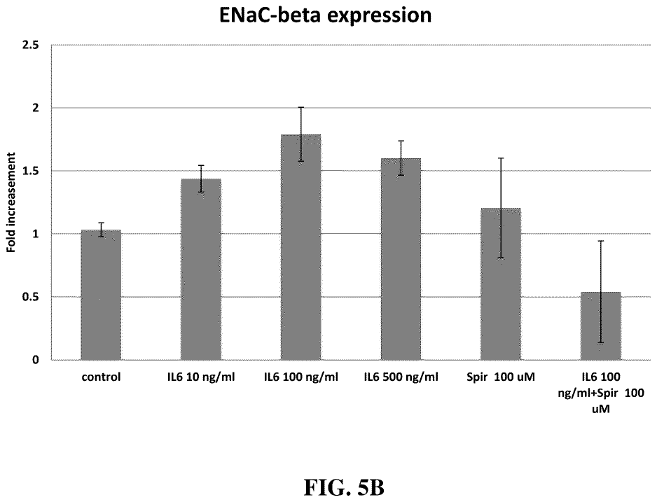

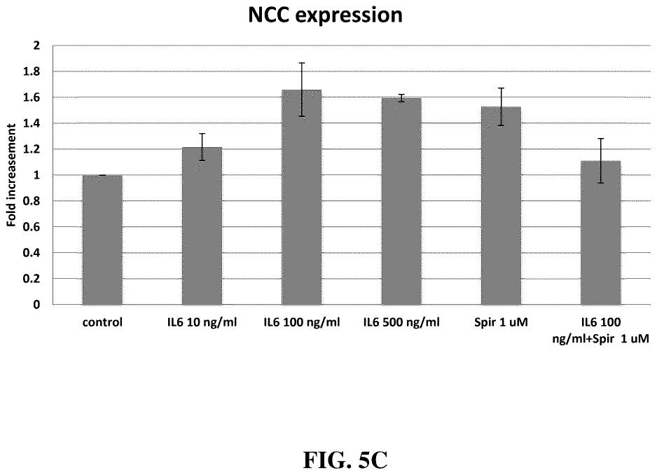

We examined the expression of NKCC2 (Na—K—Cl cotransporter 2), ENaC-beta (epithelial sodium channel, beta subunit), and NCC (sodium-chloride symporter) proteins in mouse M1 CCD cell line after treatment of IL-6 in the absence or presence of a potassium-sparing diuretic, Spironolactone. The mouse M1 CCD cells are genotypically analogous to human cells homozygous for the TMPRSS6 rs855791 major allele. As shown in FIGS. 5A, 5B, and 5C, the treatment of IL-6 increased the expression of NKCC2, ENaC-beta, and NCC. Spironolactone blocked the effect of IL-6 on the ion transporter proteins.

Increased expression of these ion transporters provides a putative mechanism for IL-6 mediated diuretic resistance.

Because the increased expression is not known to be linked to hepcidin expression, these data suggested that IL-6 antagonism could be effective in treating diuretic resistance even in patients homozygous for the TMPRSS6 rs855791 minor allele.

### Example 5: Association of IL-6 with Diuretic Response in Patients Hospitalized for Acute Heart Failure (HF)

Methods. Data from the PROTECT trial (Weatherley et al., 2010, J. Card. Fail. 16:25-35; Massie et al., 2010, N. Engl. J. Med. 363:1419-1428) was analyzed according to tertiles of IL-6. The PROTECT trial was a randomized placebo-controlled trial testing the effect of Adenosine A1-Receptor Antagonist Rolofylline on dyspnea relief, risk of worsening renal function and clinical outcomes. The key inclusion and exclusion criteria of the trial are shown below.

TABLE A

| Inclusion | Exclusion |
|---|---|
| >18 years | Acute contrast-induced nephropathy |
| History of HF >14 days with diuretic therapy | Temp >38 or sepsis requiring IV |
| Hospitalized for ADHF* requiring IV diuretic | antimicrobial treatment |
| therapy | Serum potassium <3.5 mEq/L |
| Within 24 hrs of presentation to the hospital | Ongoing or planned IV therapy for ADHF |
| Anticipated need for IV furosemide >40 mg/ | with positive inotropic agents, vasopressors, |
| day for at least 24 hr | vasodilators |
| Impaired renal function defined as creatinine | BNP <500 pg/mL or NT-pro- |
| clearance of admission between 20 and 80 | BNP <2000 pg/mL |
| mL/min (Cockcroft-Gault) | Ongoing or planned treatment with |
| Systolic blood pressure >95 mm Hg | ultrafiltration |
| | Severe pulmonary disease |
| | Clinical evidence of acute coronary |
| | syndrome in the 2 weeks before screening |
| | Hgb <8 g/dL, or Het <25%, or the |
| | need for a blood transfusion |
| | Systolic blood pressure $160 mm Hg at |
| | randomization |

*ADHF: dyspnea at rest or with minimal exertion and signs of fluid overload manifested by at least one of the following at time of randomization: JVP > 8 cm, or Pulmonary rales ≥ ⅓ up the lung fields, not clearing with cough, or ≥2 [symbol] peripheral edema, or presacral edema

In total, 2033 patients with ADHF were included in the PROTECT study. Of these patients, IL-6 was measured by Singulex in 1445 patients at admission (baseline), 1462 patients at day 2 (24 hr after baseline) and 1445 patients at day 7. Diuretic response was defined as weight change on day 4 per 40 mg of furosemide (or equivalent doses) administered from baseline to day 3. The primary endpoint of this study was all-cause mortality at 180 days.

Statistical analysis. Baseline characteristics are presented according to tertiles of IL-6. Differences between tertiles of baseline characteristics were tested using one-way analysis of variance (ANOVA), Kruskal Wallis or chi2-test where appropriate. Univariable linear regression was performed using diuretic response as the dependent variable and (log-transformed) IL-6 at baseline as the independent variable correcting for clinically relevant variables associated with diuretic response. Survival analysis was performed using Cox regression analysis correcting for clinically relevant variables and the PROTECT risk model (O'Connor et al., 2012, Eur. J. Heart Fail. 14:605-612). The PROTECT risk model includes: previous hospitalization for HF, edema, systolic blood pressure, sodium levels, BUN, creatinine and albumin at admission.

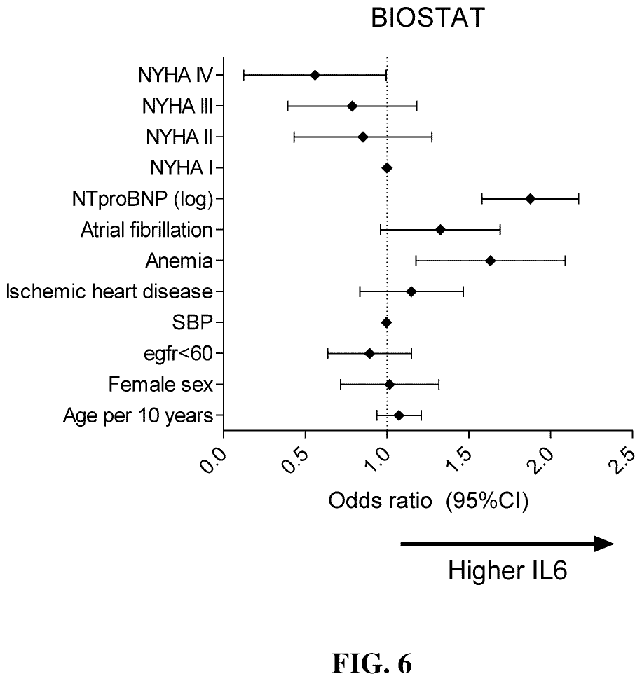

Results. The baseline characteristics of the population are described in Table 4, below. Higher levels of IL-6 at baseline are associated with higher levels of BNP, anemia, eGFR<60 and older age (FIG. 6).

TABLE 4
Baseline Characteristics

| Factor | 1st tertile | 2nd tertile | 3rd tertile | p-value |
|---|---|---|---|---|
| N | 531 | 530 | 530 | |
| Demographics | 0.66-7.8 pg/mL | 7.9-16.1 pg/mL | 16.2-274.2 pg/mL | |
| Age | 69.2 (11.5) | 70.8 (11.0) | 72.5 (10.8) | <0.001 |
| Sex | 347 (65.3%) | 349 (65.8%) | 354 (66.8%) | 0.88 |
| BMI | 28.3 (5.8) | 28.8 (6.0) | 29.1 (6.2) | 0.093 |
| eGFR | 52.5 (20.2) | 47.2 (19.3) | 44.9 (17.4) | <0.001 |
| NYHA class | | | | |
| I/II | 107 (21.0%) | 71 (14.1%) | 96 (19.2%) | 0.008 |
| III | 267 (52.4%) | 263 (52.2%) | 237 (47.4%) | |
| IV | 136 (26.7%) | 170 (33.7%) | 167 (33.4%) | |
| LVEF | 30 (25, 40) | 30 (20, 40) | 30 (22, 40) | 0.35 |
| HFpEF | 42 (15.6%) | 39 (16.3%) | 38 (14.9%) | 0.92 |
| Systolic BP | 126.2 (17.4) | 124.5 (17.5) | 123.6 (17.4) | 0.043 |
| Diastolic BP | 75.3 (11.2) | 73.5 (12.1) | 72.3 (11.9) | <0.001 |
| Heart Rate | 79.2 (14.6) | 79.4 (15.8) | 81.3 (16.0) | 0.052 |
| Respiratory rate | 20.6 (4.3) | 21.2 (4.1) | 21.7 (4.9) | <0.001 |
| Medical History | | | | |
| Atrial fibrillation | 95 (45.5%) | 92 (43.6%) | 78 (39.4%) | 0.45 |
| Valve disease | 196 (37.0%) | 195 (36.8%) | 208 (39.5%) | 0.61 |
| Mitral regurgitation | 174 (32.8%) | 178 (33.6%) | 184 (34.8%) | 0.78 |
| Aortic stenosis | 18 (3.4%) | 16 (3.0%) | 32 (6.0%) | 0.027 |
| Aortic regurgitation | 43 (8.1%) | 18 (3.4%) | 38 (7.2%) | 0.004 |
| Heart failure (HF) | 510 (96.0%) | 505 (95.3%) | 501 (94.5%) | 0.51 |
| Hospitalization for | 258 (48.6%) | 266 (50.2%) | 259 (48.9%) | 0.86 |
| HF previous year | | | | |
| No. of HF | 1.0 (1.0, 2.0) | 1.0 (1.0, 2.0) | 1.0 (1.0, 2.0) | 0.62 |
| hospitalizations, median | | | | |
| (IQR) | | | | |
| Ischemic heart disease | 362 (68.4%) | 396 (74.9%) | 365 (68.9%) | 0.037 |
| Myocardial infarction | 255 (48.3%) | 296 (56.0%) | 242 (45.7%) | 0.003 |
| Hypertension | 424 (79.8%) | 422 (79.6%) | 428 (80.8%) | 0.89 |
| Stroke or PVD | 78 (14.7%) | 102 (19.3%) | 117 (22.2%) | 0.007 |
| Anemia | 174 (36.3%) | 216 (47.2%) | 238 (50.6%) | <0.001 |
| Thyroid disease | 60 (11.3%) | 61 (11.5%) | 57 (10.8%) | 0.92 |
| Depression | 28 (5.3%) | 41 (7.7%) | 35 (6.6%) | 0.27 |
| Hyperlipidemia | 298 (56.1%) | 267 (50.5%) | 244 (46.0%) | 0.004 |
| Current smoker | 92 (17.4%) | 114 (21.6%) | 105 (19.8%) | 0.23 |
| COPD or asthma | 106 (20.0%) | 102 (19.2%) | 112 (21.2%) | 0.73 |
| Diabetes mellitus | 247 (46.5%) | 229 (43.2%) | 258 (48.7%) | 0.2 |
| History of Atrial | 260 (49.2%) | 291 (55.0%) | 310 (58.8%) | 0.007 |
| Fibrillation/Flutter | | | | |
| AICD | 73 (13.7%) | 106 (20.0%) | 68 (12.8%) | 0.002 |
| Biventricular pacemaker | 54 (10.2%) | 60 (11.3%) | 47 (8.9%) | 0.41 |
| Pacemaker | 50 (9.5%) | 66 (12.5%) | 65 (12.3%) | 0.22 |
| Medication | | | | |
| Beta-blockers | 417 (78.7%) | 396 (74.7%) | 385 (72.6%) | 0.068 |
| ACEIs | 353 (66.6%) | 312 (58.9%) | 330 (62.3%) | 0.033 |
| ARBs | 85 (16.0%) | 85 (16.0%) | 71 (13.4%) | 0.38 |
| ACE-I/ARB | 424 (80.0%) | 390 (73.6%) | 393 (74.2%) | 0.026 |
| MRA | 264 (49.8%) | 226 (42.6%) | 229 (43.2%) | 0.033 |
| Digoxin | 159 (30.0%) | 153 (28.9%) | 146 (27.5%) | 0.68 |
| Nitrates | 136 (25.7%) | 146 (27.6%) | 135 (25.5%) | 0.68 |
| Hydralazine | 17 (3.2%) | 15 (2.8%) | 13 (2.5%) | 0.76 |
| CCBs | 74 (14.0%) | 71 (13.4%) | 81 (15.3%) | 0.67 |
| Signs and symptoms | | | | |
| Orthopnea | 445 (84.3%) | 440 (84.5%) | 435 (82.5%) | 0.65 |
| Dyspnea at rest | 264 (50.6%) | 304 (59.1%) | 327 (64.8%) | <0.001 |
| (NYHA IV) | | | | |
| Angina pectoris | 134 (25.2%) | 117 (22.1%) | 111 (21.0%) | 0.24 |
| CCS Class III & IV | 48 (36.4%) | 38 (33.0%) | 29 (26.6%) | 0.27 |
| Edema | 82 (15.4%) | 151 (28.5%) | 184 (34.8%) | <0.001 |
| Jugular venous distension | 190 (39.7%) | 195 (40.5%) | 199 (41.6%) | 0.82 |
| Edema & raised JVP | 127 (26.5%) | 148 (30.7%) | 150 (31.4%) | 0.2 |
| Rales | 37 (7.0%) | 55 (10.4%) | 62 (11.7%) | 0.027 |

The association of IL-6 levels and diuretic response is shown in Table 5, below. The diuretic response was defined as weight change on day 4 per 40 mg of furosemide (or equivalent doses) administered from baseline to day 3.

TABLE 5
Diuretic Response

| | Beta | p-value |
|---|---|---|
| Univariable | 0.06 | 0.027 |
| Model 1 (age, sex) | 0.06 | 0.030 |
| Model 2 | 0.06 | 0.035 |
| (model 1 + eGFR, BMI) | | |

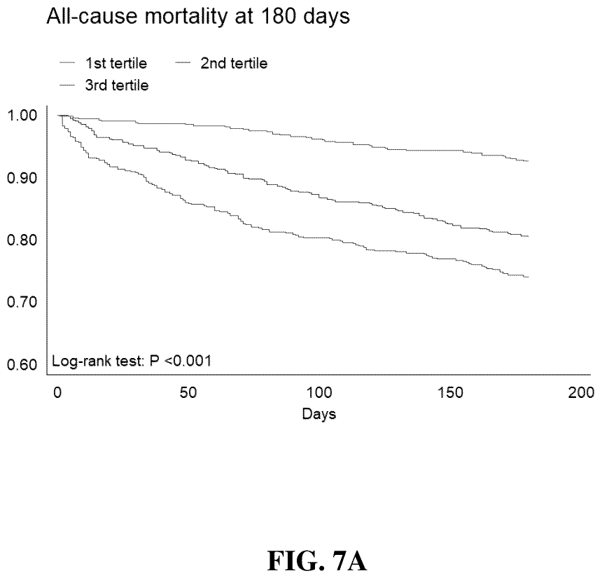

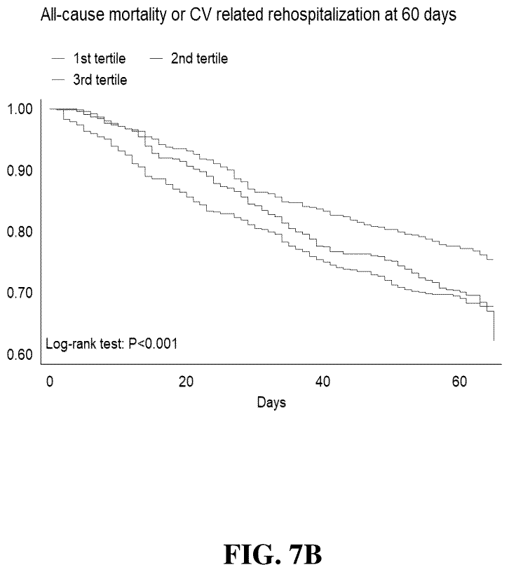

Table 6 and FIGS. 7A and 7B show the association of IL-6 levels with all-cause mortality at 180 days and the association of IL-6 levels with all-cause mortality at 60 days and/or cardiovascular related rehospitalization (CV hosp).

TABLE 6
Cox Regression Results

| | All-cause mortality | 60 days mortality |
|---|---|---|
| | at 180 days | and/or CV hosp |
| Univariable | 1.59 (1.43-1.76) | 1.14 (1.04-1.26) |
| | <0.001 | 0.007 |
| Model 1 | 1.57 (1.41-1.74) | 1.14 (1.04-1.26) |
| | <0.001 | 0.007 |
| Model 2 | 1.55 (1.39-1.72) | 1.11 (1.00-1.22) |
| | <0.001 | 0.049 |
| Model 3 | 1.50 (1.35-1.68) | 1.09 (0.99-1.21) |
| | <0.001 | 0.087 |
| PROTECT model | 1.41 (1.26-1.58) | 1.06 (0.95-1.17) |
| | <0.001 | 0.309 |

Model 1: age, sex
Model 2: model 1 + eGFR, BMI
Model 3: model 2 + BNP
PROTECT model: age, previous hospitalization for HF, edema at admission, sodium, bun (log), creatinine (log) and albumin

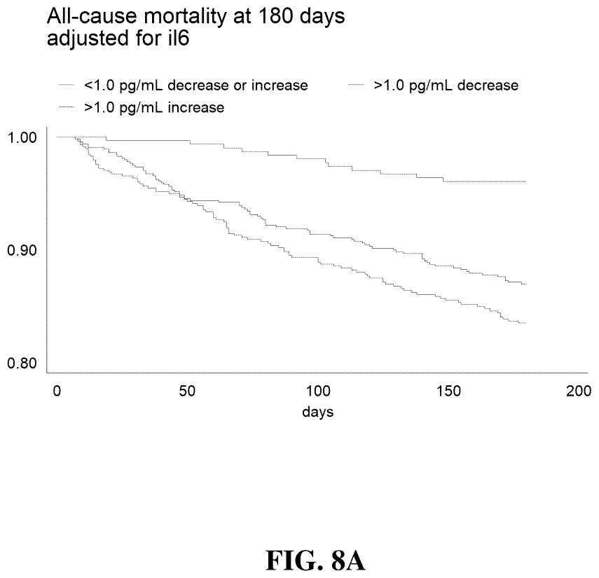

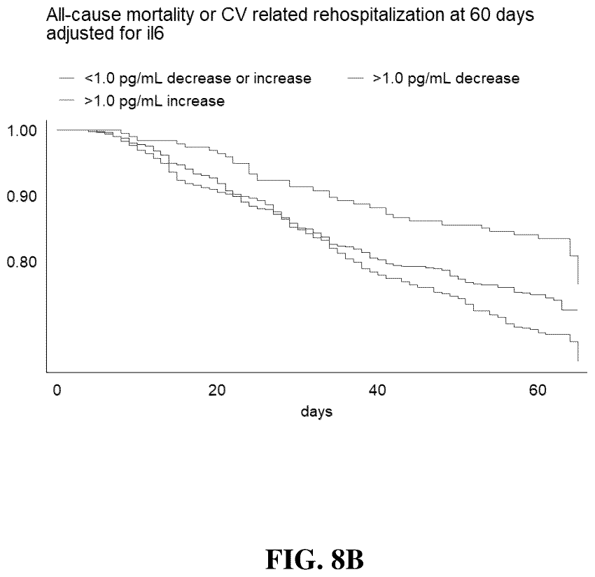

Table 7 and FIGS. 8A and 8B show that an increase in IL-6 levels at day 7 compared to the baseline predicts adverse outcomes.

TABLE 7
Cox Regression Results

| | | |
|---|---|---|
| >1 pg/mL | <1.0 pg/mL | >1.0 pg/mL |
| decrease | increase/decrease | increase |

180 days all-cause mortality

| | | | |
|---|---|---|---|
| Univariable | 2.13 (1.16-3.91) | ref | 3.01 (1.65-5.49) |
| | 0.014 | | <0.001 |
| Model 1 | 1.79 (0.96-3.32) | ref | 2.80 (1.53-5.11) |
| | 0.066 | | 0.001 |
| Model 2 | 1.80 (0.95-3.44) | ref | 2.65 (1.41-4.97) |
| | 0.073 | | 0.002 |
| Model 3 | 1.65 (0.87-3.16) | ref | 2.35 (1.25-4.44) |
| | 0.128 | | 0.008 |
| PROTECT | 1.71 (0.90-3.26) | ref | 2.38 (1.30-4.47) |
| | 0.101 | | 0.007 |

60 days mortality and/or CV hosp

| | | | |
|---|---|---|---|
| Univariable | 1.31 (0.91-1.92) | ref | 1.83 (1.26-2.65) |
| | 0.146 | | 0.001 |
| Model 1 | 1.31 (0.89-1.92) | ref | 1.81 (1.25-2.62) |
| | 0.002 | | 0.002 |
| Model 2 | 1.29 (0.88-1.92) | ref | 1.70 (1.16-2.48) |
| | 0.187 | | 0.006 |
| Model 3 | 1.28 (0.86-1.89) | ref | 1.67 (1.14-2.45) |
| | 0.223 | | 0.008 |
| PROTECT | 1.20 (0.81-1.78) | ref | 1.57 (1.07-2.30) |
| | 0.353 | | 0.020 |

Model 1: age, sex
Model 2: model 1 + eGFR, BMI
Model 3: model 2 + BNP
PROTECT model: age, previous hospitalization for HF, edema at admission, sodium, bun (log), creatinine (log) and albumin

### Example 6: Association of IL-6 Levels with Outcomes in a Worsening Heart Failure Population

Methods. Data from the BIOlogy Study to TAilored Treatment in Chronic Heart Failure (BIOSTAT-CHF) study was analyzed to investigate the association of IL-6 with outcomes in patients with worsening heart failure. In brief, BIOSTAT-CHF was a multicenter, multinational, prospective, observational study including 2516 patients from 69 centers in 11 European countries (Voors et al., 2016, Eur. J. Heart Fail. 18:716-726). We performed secondary analyses in the BIOSTAT-CHF study, excluding patients with ferritin <100 from subsequent analysis. Inclusion criteria for the index cohort include: patients with >18 years of age, having symptoms of new-onset or worsening HF, confirmed either by a LVEF of ≤40% or BNP and/or NT-proBNP plasma levels >400 pg/ml or >2,000 pg/ml, respectively. Furthermore, these patients had not been previously treated with an ACEi/ARBs and/or beta-blocker or they were receiving ≤50% of the target doses of these drugs at the time of inclusion and anticipated initiation or up-titration of ACEi/ARBs and beta-blockers. All patients needed to be treated with loop diuretics.

TABLE B
Inclusion criteria

Age > 18
Diagnosed with HF
Previous documented admission with HF
requiring diuretic treatment
Treated with furosemide >20 mg/day or equivalent
Not previously treated or receiving <50% of
target doses of ACE inhibitors/ARBs and/or
beta-blockers according to 2008 ESC guidelines
Anticipated uptitration of ACE inhibitors/
ARBs and/or beta-blockers

In total, IL-6 was measured in 2329 patients with worsening HF from the BIOSTAT-CHF study. The primary outcome of this study was a composite outcome of all-cause mortality and hospitalization for HF.

Statistical analyses. Baseline characteristics are presented according to tertiles of IL-6. Differences between tertiles of baseline characteristics were tested using one-way analysis of variance (ANOVA), Kruskal Wallis or chi2-test where appropriate. Survival analysis was performed using Cox regression analysis correcting for clinically relevant variables and the BIOSTAT-CHF risk model. The BIOSTAT-CHF risk model for all-cause mortality and/or hospitalization for heart failure includes: age, N-terminal pro-B-type natriuretic peptide (NT-proBNP), hemoglobin (Hb), the use of a beta-blocker at time of inclusion, a HF-hospitalization in year before inclusion, peripheral edema, systolic blood pressure, high-density lipoprotein cholesterol and sodium (Voors et al., 2017, Eur. J. Heart Fail. 19:627-634). We performed interaction analysis between ferritin levels and IL-6 for the primary outcome. To investigate the association of IL-6 with outcomes depending on the position of the TMPRS6 SNP (rs855791).

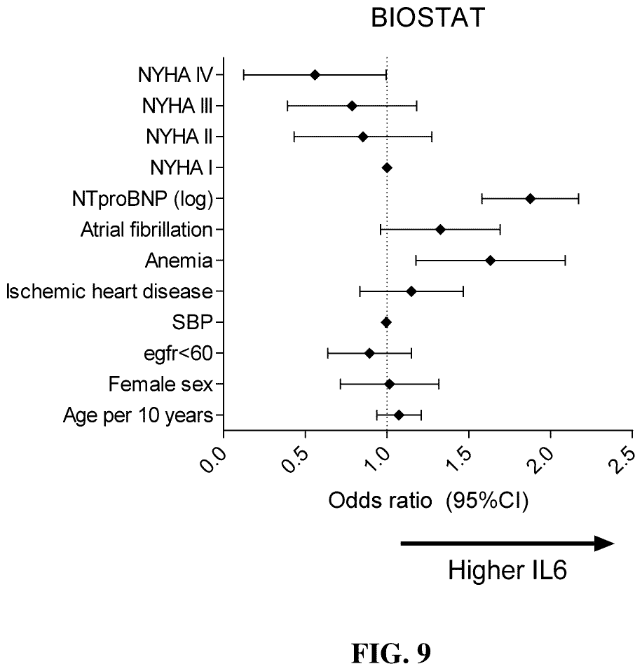

Results. The baseline characteristics of the population are described in Table 8, below. Higher levels of IL-6 at baseline are associated with higher levels of NTproBNP and Anemia (FIG. 9).

TABLE 8
Baseline Characteristics

| Factor | 1st tertile | 2nd tertile | 3rd tertile | p-value |
|---|---|---|---|---|
| N | 781 | 775 | 773 | |
| Demographics | 0.3-3.4 pg/mL | 3.5-7.8 pg/mL | 7.9-260.7 pg/mL | |
| Age | 66.3 (12.2) | 69.3 (11.7) | 70.9 (11.7) | <0.001 |
| Female | 200 (25.6%) | 200 (25.8%) | 212 (27.4%) | 0.67 |
| HF status | | | | |
| HFrEF | 607 (84.1%) | 568 (81.6%) | 524 (78.3%) | <0.001 |
| HFmrEF | 92 (12.7%) | 74 (10.6%) | 87 (13.0%) | |
| HFpEF | 23 (3.2%) | 54 (7.8%) | 58 (8.7%) | |
| BMI | 27.9 (5.1) | 27.7 (5.6) | 27.7 (5.6) | 0.77 |
| Ischemic etiology | 329 (43.2%) | 353 (46.4%) | 357 (46.7%) | 0.31 |
| NYHA | | | | |
| I | 70 (9.0%) | 82 (10.6%) | 62 (8.0%) | 0.005 |
| II | 400 (51.2%) | 355 (45.8%) | 320 (41.4%) | |
| III | 199 (25.5%) | 216 (27.9%) | 251 (32.5%) | |
| IV | 22 (2.8%) | 25 (3.2%) | 33 (4.3%) | |
| NA | 90 (11.5%) | 97 (12.5%) | 107 (13.8%) | |
| Systolic BP | 126.4 (20.0) | 124.3 (22.4) | 123.7 (23.5) | 0.034 |
| Diastolic BP | 76.7 (12.7) | 74.8 (13.4) | 73.5 (14.0) | <0.001 |
| LVEF | 30.6 (8.9) | 30.9 (11.1) | 31.3 (11.7) | 0.54 |
| Heart Rate | 76.4 (18.2) | 80.1 (18.9) | 83.6 (21.0) | <0.001 |
| Signs and symptoms | | | | |
| Peripheral edema | | | | |
| Not Present | 352 (57.2%) | 253 (39.7%) | 180 (26.4%) | <0.001 |
| Ankle | 170 (27.6%) | 188 (29.5%) | 221 (32.5%) | |
| Below Knee | 83 (13.5%) | 156 (24.5%) | 194 (28.5%) | |
| Above Knee | 10 (1.6%) | 40 (6.3%) | 86 (12.6%) | |
| Elevated JVP | | | | |
| No | 420 (76.5%) | 318 (58.9%) | 289 (53.7%) | <0.001 |
| Yes | 106 (19.3%) | 190 (35.2%) | 216 (40.1%) | |
| Uncertain | 23 (4.2%) | 32 (5.9%) | 33 (6.1%) | |
| Hepatomegaly | 75 (9.6%) | 128 (16.6%) | 132 (17.1%) | <0.001 |
| Orthopnea | 168 (21.6%) | 273 (35.3%) | 361 (46.8%) | <0.001 |
| Medical history | | | | |
| Anemia | 158 (23.6%) | 271 (38.1%) | 348 (47.0%) | <0.001 |
| Atrial fibrillation | 293 (37.5%) | 372 (48.0%) | 387 (50.1%) | <0.001 |
| Diabetes mellitus | 208 (26.6%) | 270 (34.8%) | 276 (35.7%) | <0.001 |
| COPD | 113 (14.5%) | 126 (16.3%) | 163 (21.1%) | 0.002 |
| Renal disease | 152 (19.5%) | 229 (29.5%) | 261 (33.8%) | <0.001 |
| Hypertension | 486 (62.2%) | 496 (64.0%) | 473 (61.2%) | 0.51 |
| Peripheral artery | 60 (7.7%) | 92 (11.9%) | 105 (13.6%) | <0.001 |
| disease | | | | |
| Stroke | 59 (7.6%) | 84 (10.8%) | 76 (9.8%) | 0.075 |
| PCI | 163 (20.9%) | 166 (21.4%) | 173 (22.4%) | 0.76 |
| CABG | 120 (15.4%) | 122 (15.7%) | 156 (20.2%) | 0.02 |
| Medication | | | | |
| Loop diuretics | 779 (99.7%) | 769 (99.2%) | 769 (99.5%) | 0.36 |
| ACE/ARB | 601 (77.0%) | 564 (72.8%) | 518 (67.0%) | <0.001 |
| Beta blocker | 675 (86.4%) | 654 (84.4%) | 603 (78.0%) | <0.001 |
| Aldosterone | 446 (57.1%) | 411 (53.0%) | 382 (49.4%) | 0.01 |
| antagonist | | | | |
| Laboratory | | | | |
| Hemoglobin | 13.7 (1.7) | 13.2 (1.8) | 12.7 (2.0) | <0.001 |
| Total cholesterol | 4.5 (3.7, 5.5) | 4.1 (3.4, 4.9) | 3.7 (3.1, 4.5) | <0.001 |
| AST | 24.0 (19.0, 32.0) | 25.0 (19.0, 34.0) | 27.0 (20.0, 39.0) | <0.001 |
| ALT | 25.0 (18.0, 37.0) | 25.0 (17.0, 37.0) | 23.5 (15.0, 40.0) | 0.12 |
| Sodium | 140.0 | 140.0 | 139.0 | <0.001 |
| | (138.0, 142.0) | (137.0, 142.0) | (136.0, 141.0) | |
| Potassium | 4.3 (4.0, 4.6) | 4.2 (3.9, 4.6) | 4.2 (3.8, 4.6) | <0.001 |
| HbAlc | 6.0 (5.6, 6.7) | 6.5 (5.9, 7.5) | 6.5 (5.9, 7.3) | <0.001 |
| NT-proBNP | 2661.0 | 4344.0 | 5734.5 | |
| | (1445.0, 4820.0) | (2517.0, 7837.0) | (3141.0, 11452.0) | <0.001 |
| Troponin-I | 0.0 (0.0, 0.1) | 0.0 (0.0, 0.1) | 0.0 (0.0, 0.1) | <0.001 |

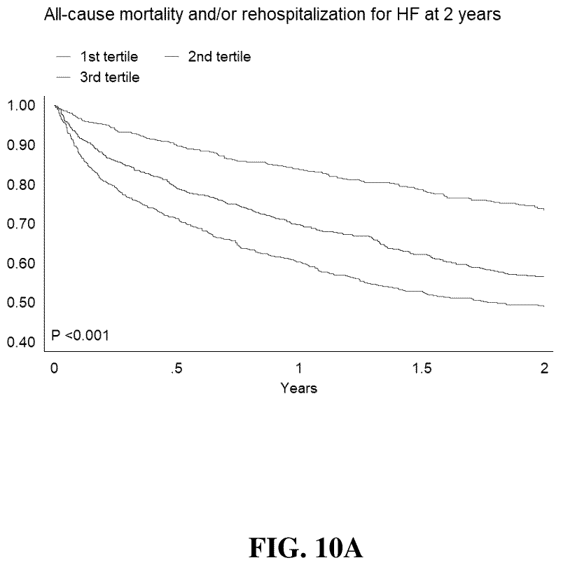

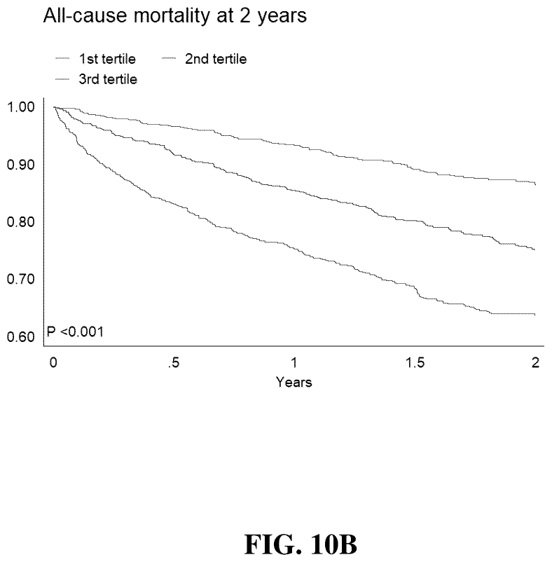

As shown in Table 9 and FIGS. 10A and 10B, levels of IL-6 measured at baseline were associated with the combined outcome of all-cause mortality and/or a hospitalization for HF as well as all-cause mortality alone at two years.

TABLE 9
Cox Regression Analyses

| | All-cause mort and/or HF | All-cause mortality |
|---|---|---|
| | hosp at 2 years HR | at 2 years HR |
| | (95% CI) p-value | (95% CI) p-value |
| Univariable | 1.38 (1.31-1.46) <0.001 | 1.42 (1.32-1.53) <0.001 |
| Model 1 | 1.34 (1.26-1.42) <0.001 | 1.48 (1.38-1.58) <0.001 |
| Model 2 | 1.25 (1.17-1.33) <0.001 | 1.34 (1.24-1.44) <0.001 |
| Model 3 | 1.24 (1.16-1.32) <0.001 | 1.33 (1.23-1.44) <0.001 |
| BIOSTAT model | 1.13 (1.04-1.19) 0.001 | 1.20 (1.11-1.31) <0.001 |

Model 1: Age, sex
Model 2: model 1 + BMI, country, hypertension (history of), diabetes (history of) and anemia
Model 3: model 2 + beta-blocker at baseline, ACEi/ARB at baseline and MRA at baseline
BIOSTAT model: age, N-terminal pro-B-type natriuretic peptide (NT-proBNP), hemoglobin (Hb), the use of a beta-blocker at time of inclusion, a HF-hospitalization in year before inclusion, peripheral edema, systolic blood pressure, high-density lipoprotein cholesterol and sodium

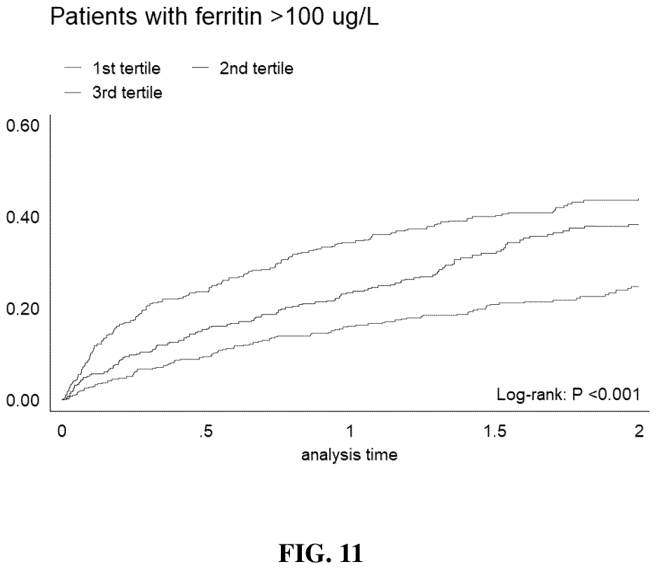

As shown in Table 10 and FIG. 11, there is no differential association of IL-6 with outcome depending on ferritin status.

TABLE 10
Cox Regression Analyses

| | Ferritin >100 ug/L | Interaction |
|---|---|---|
| | HR (95% CI) p-value | ferritin *IL-6 |
| Univariable | 1.39 (1.27-1.52) | 0.610 |
| | <0.001 | |
| Model 1 | 1.35 (1.23-1.48) | |
| | <0.001 | |
| Model 2 | 1.26 (1.14-1.40) | |
| | <0.001 | |
| Model 3 | 1.26 (1.14-1.39) | |
| | <0.001 | |
| BIOSTAT model | 1.11 (0.99-1.22) | |
| | 0.052 | |

Model 1: Age, sex
Model 2: model 1 + BMI, country, hypertension (history of), diabetes (history of) and anemia
Model 3: model 2 + beta-blocker at baseline, ACEi/ARB at baseline and MRA at baseline
BIOSTAT model: age, N-terminal pro-B-type natriuretic peptide (NT-proBNP), hemoglobin (Hb), the use of a beta-blocker at time of inclusion, a HF-hospitalization in year before inclusion, peripheral edema, systolic blood pressure, high-density lipoprotein cholesterol and sodium

As shown in Table 11, there was no differential association of IL-6 with outcome depending on TMPRSS6 genotype.

TABLE 11
IL-6 according to TMPRSS6 allele

| | HR (95% CI) |
|---|---|
| TMPRSS6 | p-value |
| AA | 1.50 (1.19-1.89) |
| (n = 191) | 0.001 |
| AG | 1.34 (1.17-1.54) |
| (n = 579) | <0.001 |
| GG | 1.54 (1.32-1.79) |
| (n = 409) | <0.001 |

### INCORPORATION BY REFERENCE

The disclosures of each and every patent, patent application, and publication cited herein are hereby incorporated herein by reference in their entirety.

### EQUIVALENTS

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1.-56. (canceled)

57. A method of reducing the risk of hospitalization for heart failure in a patient with heart failure, comprising:
administering a therapeutically effective amount of an IL-6 antagonist.

58. The method of claim 57, wherein the patient has left ventricular heart failure.

59. The method of claim 57, wherein the patient has heart failure with preserved ejection fraction (HFpEF).

60. The method of claim 57, wherein the patient has heart failure with mildly reduced ejection fraction (HFmrEF).

61. The method of any one of claim 57, wherein the patient has heart failure with reduced ejection fraction (HFrEF).

62. The method of claim 57, wherein the patient has at least one copy of the TMPRSS6 rs855791 major allele.

63. The method of claim 57, wherein the IL-6 antagonist is an anti-IL-6 antibody, or antigen-binding fragment or derivative thereof.

64. The method of claim 63, wherein the anti-IL-6 antibody or antigen-binding fragment or derivative comprises all six variable region CDRs of MED5117.

65. The method of claim 64, wherein the antibody comprises the VH and VL of MED5117.

66. The method of claim 64, wherein the antibody is MED5117.

67. The method of claim 57, wherein the anti-IL-6 antibody is co-administered with one or more therapeutic agents selected from the group of angiotensin converting enzyme inhibitor (ACE-I), angiotensin receptor blocker (ARB), and beta-blocker.

68. The method of claim 57, wherein the anti-IL-6 antibody is co-administered with a loop diuretic.


SEQUENCE LISTING (16 sequences)

SEQ ID NO: 1 - protein, 824 aa, Homo sapiens
MPVAEAPQVAGGQGDGGDGEEAEPEGMFKACEDSKRKARGYLRLVPLFVLLALLVLASAGVLLWYFLGYKAEVMVSQVYSGSLRVLNRHFSQDLTRRESSAFRSETAKAQKMLKELITSTRLGTYYNSSSVYSFGEGPLTCFFWFILQIPEHRRLMLSPEVVQALLVEELLSTVNSSAAVPYRAEYEVDPEGLVILEASVKDIAALNSTLGCYRYSYVGQGQVLRLKGPDHLASSCLWHLQGPKDLMLKLRLEWTLAECRDRLAMYDVAGPLEKRLITSVYGCSRQEPVVEVLASGAIMAVVWKKGLHSYYDPFVLSVQPVVFQACEVNLTLDNRLDSQGVLSTPYFPSYYSPQTHCSWHLTVPSLDYGLALWFDAYALRRQKYDLPCTQGQWTIQNRRLCGLRILQPYAERIPVVATAGITINFTSQISLTGPGVRVHYGLYNQSDPCPGEFLCSVNGLCVPACDGVKDCPNGLDERNCVCRATFQCKEDSTCISLPKVCDGQPDCLNGSDEEQCQEGVPCGTFTFQCEDRSCVKKPNPQCDGRPDCRDGSDEEHCDCGLQGPSSRIVGGAVSSEGEWPWQASLQVRGRHICGGALIADRWVITAAHCFQEDSMASTVLWTVFLGKVWQNSRWPGEVSFKVSRLLLHPYHEEDSHDYDVALLQLDHPVVRSAAVRPVCLPARSHFFEPGLHCWITGWGALREGALRADAVALFYGWRNQGSETCCCPISNALQKADVQLIPQDLCSEVYRYQVTPRMLCAGYRKGKKDACQGDSGGPLVCKALSGRWFLAGLVSWGLGCGRPNYFGVYTRITGVISWIQQVVT

SEQ ID NO: 2 - protein, 824 aa, Homo sapiens
MPVAEAPQVAGGQGDGGDGEEAEPEGMFKACEDSKRKARGYLRLVPLFVLLALLVLASAGVLLWYFLGYKAEVMVSQVYSGSLRVLNRHFSQDLTRRESSAFRSETAKAQKMLKELITSTRLGTYYNSSSVYSFGEGPLTCFFWFILQIPEHRRLMLSPEVVQALLVEELLSTVNSSAAVPYRAEYEVDPEGLVILEASVKDIAALNSTLGCYRYSYVGQGQVLRLKGPDHLASSCLWHLQGPKDLMLKLRLEWTLAECRDRLAMYDVAGPLEKRLITSVYGCSRQEPVVEVLASGAIMAVVWKKGLHSYYDPFVLSVQPVVFQACEVNLTLDNRLDSQGVLSTPYFPSYYSPQTHCSWHLTVPSLDYGLALWFDAYALRRQKYDLPCTQGQWTIQNRRLCGLRILQPYAERIPVVATAGITINFTSQISLTGPGVRVHYGLYNQSDPCPGEFLCSVNGLCVPACDGVKDCPNGLDERNCVCRATFQCKEDSTCISLPKVCDGQPDCLNGSDEEQCQEGVPCGTFTFQCEDRSCVKKPNPQCDGRPDCRDGSDEEHCDCGLQGPSSRIVGGAVSSEGEWPWQASLQVRGRHICGGALIADRWVITAAHCFQEDSMASTVLWTVFLGKVWQNSRWPGEVSFKVSRLLLHPYHEEDSHDYDVALLQLDHPVVRSAAVRPVCLPARSHFFEPGLHCWITGWGALREGALRADAVALFYGWRNQGSETCCCPISNALQKVDVQLIPQDLCSEVYRYQVTPRMLCAGYRKGKKDACQGDSGGPLVCKALSGRWFLAGLVSWGLGCGRPNYFGVYTRITGVISWIQQVVT

SEQ ID NO: 3 - DNA, 3196 bp, Homo sapiens
  source 1..3196: mol_type = genomic DNA
ggacaaacagaggctcctgaggcctgtgtgcaggcccggcacctatctgccgctcccaaaggatgcccgtggccgaggccccccaggtggctggcgggcagggggacggaggtgatggcgaggaagcggagccggaggggatgttcaaggcctgtgaggactccaagagaaaagcccggggctacctccgcctggtgcccctgtttgtgctgctggccctgctcgtgctggcttcggcgggggtgctactctggtatttcctagggtacaaggcggaggtgatggtcagccaggtgtactcaggcagtctgcgtgtactcaatcgccacttctcccaggatcttacccgccgggaatctagtgccttccgcagtgaaaccgccaaagcccagaagatgctcaaggagctcatcaccagcacccgcctgggaacttactacaactccagctccgtctattcctttggggagggacccctcacctgcttcttctggttcattctccaaatccccgagcaccgccggctgatgctgagccccgaggtggtgcaggcactgctggtggaggagctgctgtccacagtcaacagctcggctgccgtcccctacagggccgagtacgaagtggaccccgagggcctagtgatcctggaagccagtgtgaaagacatagctgcattgaattccacgctgggttgttaccgctacagctacgtgggccagggccaggtcctccggctgaaggggcctgaccacctggcctccagctgcctgtggcacctgcagggccccaaggacctcatgctcaaactccggctggagtggacgctggcagagtgccgggaccgactggccatgtatgacgtggccgggcccctggagaagaggctcatcacctcggtgtacggctgcagccgccaggagcccgtggtggaggttctggcgtcgggggccatcatggcggtcgtctggaagaagggcctgcacagctactacgaccccttcgtgctctccgtgcagccggtggtcttccaggcctgtgaagtgaacctgacgctggacaacaggctcgactcccagggcgtcctcagcaccccgtacttccccagctactactcgccccaaacccactgctcctggcacctcacggtgccctctctggactacggcttggccctctggtttgatgcctatgcactgaggaggcagaagtatgatttgccgtgcacccagggccagtggacgatccagaacaggaggctgtgtggcttgcgcatcctgcagccctacgccgagaggatccccgtggtggccacggccgggatcaccatcaacttcacctcccagatctccctcaccgggcccggtgtgcgggtgcactatggcttgtacaaccagtcggacccctgccctggagagttcctctgttctgtgaatggactctgtgtccctgcctgtgatggggtcaaggactgccccaacggcctggatgagagaaactgcgtttgcagagccacattccagtgcaaagaggacagcacatgcatctcactgcccaaggtctgtgatgggcagcctgattgtctcaacggcagcgacgaagagcagtgccaggaaggggtgccatgtgggacattcaccttccagtgtgaggaccggagctgcgtgaagaagcccaacccgcagtgtgatgggcggcccgactgcagggacggctcggatgaggagcactgtgactgtggcctccagggcccctccagccgcattgttggtggagctgtgtcctccgagggtgagtggccatggcaggccagcctccaggttcggggtcgacacatctgtgggggggccctcatcgctgaccgctgggtgataacagctgcccactgcttccaggaggacagcatggcctccacggtgctgtggaccgtgttcctgggcaaggtgtggcagaactcgcgctggcctggagaggtgtccttcaaggtgagccgcctgctcctgcacccgtaccacgaagaggacagccatgactacgacgtggcgctgctgcagctcgaccacccggtggtgcgctcggccgccgtgcgccccgtctgcctgcccgcgcgctcccacttcttcgagcccggcctgcactgctggattacgggctggggcgccttgcgcgagggcgccctacgggcggatgctgtggccctattttatggatggagaaaccaaggctcagagacatgttgctgccccatcagcaacgctctgcagaaagtggatgtgcagttgatcccacaggacctgtgcagcgaggtctatcgctaccaggtgacgccacgcatgctgtgtgccggctaccgcaagggcaagaaggatgcctgtcagggtgactcaggtggtccgctggtgtgcaaggcactcagtggccgctggttcctggcggggctggtcagctggggcctgggctgtggccggcctaactacttcggcgtctacacccgcatcacaggtgtgatcagctggatccagcaagtggtgacctgaggaactgcccccctgcaaagcagggcccacctcctggactcagagagcccagggcaactgccaagcagggggacaagtattctggcggggggtgggggagagagcaggccctgtggtggcaggaggtggcatcttgtctcgtccctgatgtctgctccagtgatggcaggaggatggagaagtgccagcagctgggggtcaagacgtcccctgaggacccaggcccacacccagcccttctgcctcccaattctctctcctccgtccccttcctccactgctgcctaatgcaaggcagtggctcagcagcaagaatgctggttctacatcccgaggagtgtctgaggtgcgccccactctgtacagaggctgtttgggcagccttgcctccagagagcagattccagcttcggaagcccctggtctaacttgggatctgggaatggaaggtgctcccatcggaggggaccctcagagccctggagactgccaggtgggcctgctgccactgtaagccaaaaggtggggaagtcctgactccagggtccttgccccacccctgcctgccacctgggccctcacagcccagaccctcactgggaggtgagctcagctgccctttggaataaagctgcctgatccaaaaaaaaaaaaaaaaa

SEQ ID NO: 4 - DNA, 3196 bp, Homo sapiens
  source 1..3196: mol_type = genomic DNA
ggacaaacagaggctcctgaggcctgtgtgcaggcccggcacctatctgccgctcccaaaggatgcccgtggccgaggccccccaggtggctggcgggcagggggacggaggtgatggcgaggaagcggagccggaggggatgttcaaggcctgtgaggactccaagagaaaagcccggggctacctccgcctggtgcccctgtttgtgctgctggccctgctcgtgctggcttcggcgggggtgctactctggtatttcctagggtacaaggcggaggtgatggtcagccaggtgtactcaggcagtctgcgtgtactcaatcgccacttctcccaggatcttacccgccgggaatctagtgccttccgcagtgaaaccgccaaagcccagaagatgctcaaggagctcatcaccagcacccgcctgggaacttactacaactccagctccgtctattcctttggggagggacccctcacctgcttcttctggttcattctccaaatccccgagcaccgccggctgatgctgagccccgaggtggtgcaggcactgctggtggaggagctgctgtccacagtcaacagctcggctgccgtcccctacagggccgagtacgaagtggaccccgagggcctagtgatcctggaagccagtgtgaaagacatagctgcattgaattccacgctgggttgttaccgctacagctacgtgggccagggccaggtcctccggctgaaggggcctgaccacctggcctccagctgcctgtggcacctgcagggccccaaggacctcatgctcaaactccggctggagtggacgctggcagagtgccgggaccgactggccatgtatgacgtggccgggcccctggagaagaggctcatcacctcggtgtacggctgcagccgccaggagcccgtggtggaggttctggcgtcgggggccatcatggcggtcgtctggaagaagggcctgcacagctactacgaccccttcgtgctctccgtgcagccggtggtcttccaggcctgtgaagtgaacctgacgctggacaacaggctcgactcccagggcgtcctcagcaccccgtacttccccagctactactcgccccaaacccactgctcctggcacctcacggtgccctctctggactacggcttggccctctggtttgatgcctatgcactgaggaggcagaagtatgatttgccgtgcacccagggccagtggacgatccagaacaggaggctgtgtggcttgcgcatcctgcagccctacgccgagaggatccccgtggtggccacggccgggatcaccatcaacttcacctcccagatctccctcaccgggcccggtgtgcgggtgcactatggcttgtacaaccagtcggacccctgccctggagagttcctctgttctgtgaatggactctgtgtccctgcctgtgatggggtcaaggactgccccaacggcctggatgagagaaactgcgtttgcagagccacattccagtgcaaagaggacagcacatgcatctcactgcccaaggtctgtgatgggcagcctgattgtctcaacggcagcgacgaagagcagtgccaggaaggggtgccatgtgggacattcaccttccagtgtgaggaccggagctgcgtgaagaagcccaacccgcagtgtgatgggcggcccgactgcagggacggctcggatgaggagcactgtgactgtggcctccagggcccctccagccgcattgttggtggagctgtgtcctccgagggtgagtggccatggcaggccagcctccaggttcggggtcgacacatctgtgggggggccctcatcgctgaccgctgggtgataacagctgcccactgcttccaggaggacagcatggcctccacggtgctgtggaccgtgttcctgggcaaggtgtggcagaactcgcgctggcctggagaggtgtccttcaaggtgagccgcctgctcctgcacccgtaccacgaagaggacagccatgactacgacgtggcgctgctgcagctcgaccacccggtggtgcgctcggccgccgtgcgccccgtctgcctgcccgcgcgctcccacttcttcgagcccggcctgcactgctggattacgggctggggcgccttgcgcgagggcgccctacgggcggatgctgtggccctattttatggatggagaaaccaaggctcagagacatgttgctgccccatcagcaacgctctgcagaaagtggatgtgcagttgatcccacaggacctgtgcagcgaggtctatcgctaccaagtgacgccacgcatgctgtgtgccggctaccgcaagggcaagaaggatgcctgtcagggtgactcaggtggtccgctggtgtgcaaggcactcagtggccgctggttcctggcggggctggtcagctggggcctgggctgtggccggcctaactacttcggcgtctacacccgcatcacaggtgtgatcagctggatccagcaagtggtgacctgaggaactgcccccctgcaaagcagggcccacctcctggactcagagagcccagggcaactgccaagcagggggacaagtattctggcggggggtgggggagagagcaggccctgtggtggcaggaggtggcatcttgtctcgtccctgatgtctgctccagtgatggcaggaggatggagaagtgccagcagctgggggtcaagacgtcccctgaggacccaggcccacacccagcccttctgcctcccaattctctctcctccgtccccttcctccactgctgcctaatgcaaggcagtggctcagcagcaagaatgctggttctacatcccgaggagtgtctgaggtgcgccccactctgtacagaggctgtttgggcagccttgcctccagagagcagattccagcttcggaagcccctggtctaacttgggatctgggaatggaaggtgctcccatcggaggggaccctcagagccctggagactgccaggtgggcctgctgccactgtaagccaaaaggtggggaagtcctgactccagggtccttgccccacccctgcctgccacctgggccctcacagcccagaccctcactgggaggtgagctcagctgccctttggaataaagctgcctgatccaaaaaaaaaaaaaaaaa

SEQ ID NO: 5 - protein, 274 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPSFRTPIQEEQADAHSTLAKI

SEQ ID NO: 6 - DNA, 1201 bp, Homo sapiens
  source 1..1201: mol_type = genomic DNA
aatattagagtctcaacccccaataaatataggactggagatgtctgaggctcattctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaaaaaaaaaaaaaaaaaaa

SEQ ID NO: 7 - protein, 468 aa, Homo sapiens
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 8 - protein, 918 aa, Homo sapiens
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 9 - protein, 5 aa, synthetic construct
  REGION 1..5: note = Description of Artificial Sequence: Synthetic peptide
SNYMI

SEQ ID NO: 10 - protein, 17 aa, synthetic construct
  REGION 1..17: note = Description of Artificial Sequence: Synthetic peptide
DLYYYAGDTYYADSVKG

SEQ ID NO: 11 - protein, 11 aa, synthetic construct
  REGION 1..11: note = Description of Artificial Sequence: Synthetic peptide
WADDHPPWIDL

SEQ ID NO: 12 - protein, 11 aa, synthetic construct
  REGION 1..11: note = Description of Artificial Sequence: Synthetic peptide
RASQGISSWLA

SEQ ID NO: 13 - protein, 7 aa, synthetic construct
  REGION 1..7: note = Description of Artificial Sequence: Synthetic peptide
KASTLES

SEQ ID NO: 14 - protein, 8 aa, synthetic construct
  REGION 1..8: note = Description of Artificial Sequence: Synthetic peptide
QQSWLGGS

SEQ ID NO: 15 - protein, 450 aa, synthetic construct
  REGION 1..450: note = Description of Artificial Sequence: Synthetic polypeptide
EVQLVESGGGLVQPGGSLRLSCAASGFTISSNYMIWVRQAPGKGLEWVSDLYYYAGDTYYADSVKGRFTMSRDISKNTVYLQMNSLRAEDTAVYYCARWADDHPPWIDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 213 aa, synthetic construct
  REGION 1..213: note = Description of Artificial Sequence: Synthetic polypeptide
DIQMTQSPSTLSASVGDRVTITCRASQGISSWLAWYQQKPGKAPKVLIYKASTLESGVPSRFSGSGSGTEFTLTISSLQPDDFATYYCQQSWLGGSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC